(19) United States
(12) Patent Application Publication
(10) Pub. No.: US 2005/0090559 A1
(43) Pub. Date: Apr. 28, 2005

(54) Heterocyclically-substituted pentanol derivatives, process for their production and their use as anti-inflammatory agents

(76) Inventors: Markus Berger, Berlin (DE); Stefan Baeurle, Berlin (DE); Hartmut Rehwinkel, Berlin (DE); Norbert Schmees, Berlin (DE); Heike Schaecke, Berlin (DE); Manfred Lehmann, Berlin (DE); Konrad Krolikiewicz, Berlin (DE); Arndt Schottelius, Belvedere (CA); Duy Nguyen, Berlin (DE); Anne Mengel, Berlin (DE); Stefan Jaroch, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/882,103

(22) Filed: Jul. 1, 2004

Related U.S. Application Data
(60) Provisional application No. 60/483,907, filed on Jul. 2, 2003.
(60) Provisional application No. 60/510,085, filed on Oct. 10, 2003.

(30) Foreign Application Priority Data
Jul. 1, 2003 (DE) 103 30 358.8
Oct. 6, 2003 (DE) 103 46 939.7

(51) Int. Cl. A61K 31/495; A61K 31/135; A61K 31/275

(52) U.S. Cl. 514/651; 564/357; 514/523

(57) ABSTRACT

The invention relates to pentanol derivatives of general formula I 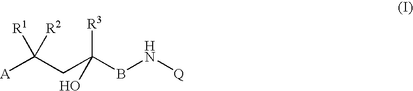 that are substituted by quinazoline, quinoxaline, cinnoline, indazole, phthalazine, naphthyridine, benzothiazole, dihydroindolone, dihydroisoindolone, benzimidazole or indole, a process for their production and their use as anti-inflammatory agents.

The invention relates to heterocyclically-substituted pentanol derivatives, in particular pentanol derivatives that are substituted by quinazoline, quinoxaline, cinnoline, indazole, phthalazine, naphthyridine, benzothiazole, dihydroindolone, dihydroisoindolone, benzimidazole or indole, process for their production and their use as anti-inflammatory agents.

From the prior art of WO 00/32584, DE 100 38 639 A1 and WO 02/10143, anti-inflammatory agents of the general formula 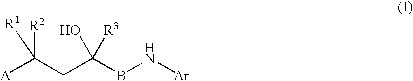
are known, whereby the Ar radical comprises phthalides, thiophthalides, benzoxazinones or phthalazinones. In the experiment, these compounds show dissociations of action between anti-inflammatory and undesirable metabolic actions and are superior to the previously described nonsteroidal glucocorticoids or exhibit at least just as good an action.

In addition, compounds in which Q represents an aromatic carbocyclic radical are known from WO03/059899.

The selectivity of the compounds of the prior art compared to the other steroid receptors still requires improvement, however.

It was therefore the object of this invention to make available compounds whose selectivity is improved compared to the other steroid receptors.

This object is achieved by the compounds according to the claims.

This invention therefore relates to compounds of general formula I 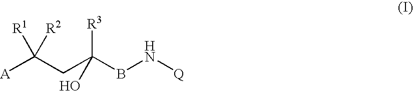
in which
A stands for an aryl, a benzyl or a phenethyl group, whereby the aryl, benzyl or phenethyl group optionally can be substituted by one or more radicals from the group C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—,—O—(CH₂)ₙ—CH₂—, —O—CH═CH—, or —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom mean the chain of a C₃-C₆-cycloalkyl ring,
R³ means a C₁-C₈-alkyl group that is optionally substituted, independently of one another, by one or more groups selected from halogen, hydroxy or C₁-C₃-alkoxy, or an optionally partially or completely fluorinated C₁-C₃-alkyl group, an optionally substituted group that is selected from C₂-C₆-alkenyl, C₂-C₆-alkinyl, C₃-C₈-cycloalkyl, C₃-C₇-heterocyclyl, aryl, heteroaryl, (C₁-C₈-alkyl)C₃-C₈-cycloalkyl, (C₁-C₈-alkyl)aryl, or (C₁-C₈-alkyl)heteroaryl,
B means a methylene group or a carbonyl group that is optionally substituted by a methyl or ethyl group, and
Q means a quinazolinyl, quinoxalinyl, cinnolinyl, indazolyl, phthalazinyl, naphthyridinyl, benzothiazolyl, dihydroindolonyl, dihydroisoindolonyl, benzimidazole or indolyl group that is linked via any position and that optionally can be substituted by one or more radicals from the group C₁-C₅-alkyl, C₁-C₅-alkoxy, C₁-C₅-alkylthio, C₁-C₅-perfluoroalkyl, halogen, hydroxy, cyano, nitro, or NR⁴R⁵, whereby R⁴ and R⁵, independently of one another, can be hydrogen, C₁-C₅-alkyl or (CO)—C₁-C₅-alkyl, whereby phthalazinones are excluded,
   as well as their racemates or separately present stereoisomers, and optionally their physiologically compatible salts.

In view of the prior art WO 98/54159 and WO 00/32584, phthalazinones were excluded. They are produced by the definition in claim 1 of Q=phthalazine in combination with the possible substituent hydroxy, since hydroxyphthalazines are at a tautomeric equilibrium with phthalazinones.

Compounds of general formula I in which
A stands for an aryl group, a benzyl group or a phenethyl group, whereby the aryl, benzyl or phenethyl group optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂—, —O—CH═CH—, or —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom of the chain mean a C₃-C₆-cycloalkyl ring,
R³ means a C₁-C₃-alkyl group or an optionally partially or completely fluorinated C₁-C₃-alkyl group,
B means a methylene group or a carbonyl group that is optionally substituted by a methyl or ethyl group, and
Q means a quinazolinyl, quinoxalinyl, cinnolinyl, indazolyl, phthalazinyl, naphthyridinyl or benzothiazolyl group that is linked via any position and that optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro or NR⁴R⁵, whereby R⁴ and R⁵, independently of one another, can be hydrogen, C₁-C₅-alkyl or (CO)—C₁-C₅-alkyl, whereby phthalazinones are excluded,
   as well as their racemates or separately present stereoisomers, and optionally their physiologically compatible salts are one aspect of the invention.

Compounds of general formula I in which
A stands for an aryl group, a benzyl group or a phenethyl group, whereby the aryl, benzyl or phenethyl group optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂, —O—CH═CH—, —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom of the chain mean a C₃-C₆-cycloalkyl ring,
R³ means a C₁-C₃-alkyl group or an optionally partially or completely fluorinated C₁-C₃-alkyl group,
B means a methylene group or a carbonyl group that is optionally substituted by a methyl or ethyl group, and
Q means a quinazolinyl, quinoxalinyl, cinnolinyl, indazolyl, naphthyridinyl or benzothiazolyl group that is linked via any position, which optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro or NR⁴R⁵, whereby R⁴ and R⁵, independently of one another, can be hydrogen, C₁-C₅-alkyl or (CO)—C₁-C₅-alkyl,
   as well as their racemates or separately present stereoisomers, and optionally their physiologically compatible salts are a subject of the invention.

Compounds of general formula I in which R³ represents a C₁-C₈-alkyl group that is optionally substituted, independently of one another, by one or more groups selected from halogen, hydroxy or C₁-C₃-alkoxy, or an optionally partially or completely fluorinated C₁-C₃-alkyl group are a subject of the invention.

Compounds of general formula I in which R³ is a C₁-C₃-alkyl group or an optionally partially or completely fluorinated C₁-C₃-alkyl group are a special subject of the invention. Preferred are the CF₃ group and the C₂F₅ group.

The C₁-C₅- or C₁-C₈-alkyl groups can be straight-chain or branched and can stand for a methyl, ethyl, n-propyl, iso-propyl, n-butyl, iso-butyl, tert-butyl, or n-pentyl, 2,2-dimethylpropyl, 2-methylbutyl or 3-methylbutyl group. A methyl or ethyl group is preferred.

For a partially or completely fluorinated C₁-C₃-alkyl group, for example, the following partially or completely fluorinated straight-chain or branched groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, and pentafluoroethyl. Of the latter, the trifluoromethyl group or the pentafluoroethyl group is preferred.

A C₁-C₅-perfluoroalkyl group is defined as a completely fluorinated straight-chain or branched alkyl group, such as, e.g., CF₃, C₂F₅, C₃F₇, C₄F₉, or C₅F₁₁.

Alkyl radicals R¹ and R² together with the carbon atom of the chain can form a 3- to 6-membered ring. The methyl group or the ethyl group is preferred for R¹ and R².

As alkyl radicals R⁴ and R⁵, C₁-C₃-alkyl is preferred, whereby the C₁-C₃-alkyl group can be straight-chain or branched.

The C₁-C₅-alkoxy groups in A and Q can be straight-chain or branched and stand for, for example, a methoxy, ethoxy, n-propoxy, iso-propoxy, n-butoxy, iso-butoxy, tert.-butoxy or n-pentoxy, 2,2-dimethylpropoxy, 2-methylbutoxy or 3-methylbutoxy group. A methoxy or ethoxy group is preferred.

The C₁-C₅-alkylthio groups in A and Q can be straight-chain or branched and stand for a methylthio, ethylthio, n-propylthio, iso-propylthio, n-butylthio, iso-butylthio, tert.-butylthio or n-pentylthio, 2,2-dimethylpropylthio, 2-methylbutylthio or 3-methylbutylthio group. A methylthio or ethylthio group is preferred.

The designation halogen atom or halogen means a fluorine, chlorine, bromine or iodine atom. A fluorine, chlorine or bromine atom is preferred.

The NR⁴R⁵ group can mean, for example, NH₂, N(H)CH₃, N(CH₃)₂, N(H)(CO)CH₃, N(CH₃)(CO)CH₃, N[(CO)CH₃]₂, N(H)CO₂CH₃, N(CH₃)CO₂CH₃, or N(CO₂CH₃)₂.

As acyl radicals R⁴ and R⁵, (CO)—C₁-C₃-alkyl is preferred, whereby the C₁-C₃-alkyl radical can be straight-chain or branched.

The C₂-C₆- or C₂-C₅-alkenyl group is straight-chain or branched; for example, vinyl, propenyl, isopropenyl, butenyl, or isobutenyl is suitable.

The C₂-C₆- or C₂-C₅-alkinyl group is straight-chain or branched; for example, C≡C, propinyl, isopropinyl, butinyl, or isobutinyl is suitable.

For a cycloalkyl group, for example, cyclopropyl, cyclobutyl, cyclopentyl, cyclohexyl, cycloheptyl or cyclooctyl is considered.

The C₁-C₈-alkyl(C₃-C₈)cycloalkyl group can be, for example, cyclobutylmethyl, cyclopentylmethyl, cyclohexylmethyl, or cycloheptylmethyl. The linkage with the chain is carried out via the alkyl group.

The heterocyclyl group is not aromatic and can be, for example, pyrrolidine, imidazolidine, pyrazolidine, or piperidine.

For an aryl group, phenyl and naphthyl are considered, and for (C₁-C₈)alkylaryl, benzyl and homobenzyl are considered. If the aryl group stands for A, the phenyl group is preferred.

Heteroaryl comprises, for example, furanyl, thienyl, thiazolyl, oxazolyl, imidazolyl, triazolyl, pyridyl and pyrimidyl.

(C₁-C₈-Alkyl)heteroaryl comprises all combinations from the definition of alkyl given above with monocyclic aromatic heterocyclic compounds, in particular the heterocyclic compounds that are mentioned by name. The linkage with the chain is carried out via the alkyl group, which in turn is linked to any chemically possible position of the heterocyclic compound.

The substituents of groups in R³ can be C₁-C₆-alkyl, C₂-C₆-alkenyl, C₂-C₆-alkinyl, C₁-C₆-alkoxy, halogen, hydroxy, and NR⁴R⁵.

In the ring, the substituted aryl, benzyl or phenethyl groups carry 1-4 or 1-3 substituents, preferably 2 substituents.

The substituents for A can be selected, independently of one another, from the group that consists of C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂—, —O—CH═CH—, —(CH₂)ₙ₊₂—, (whereby n=1 or 2, and the terminal oxygen atoms and/or carbon atoms are linked to directly adjacent ring-carbon atoms), or NR⁴R⁵ (whereby R⁴ and R⁵, independently of one another, are hydrogen, C₁-C₅-alkyl or (CO)—C₁-C₅-alkyl).

C₁-C₅-Alkyl, C₁-C₅-alkoxy, C₁-C₅-alkylthio, C₁-C₅-perfluoroalkyl, halogen, hydroxy, —O—(CH₂)ₙ—O—, —O—(CH₂)ₙ—CH₂—, —O—CH═CH— and —(CH₂)ₙ₊₂— are preferred.

C₁-C₅-Alkyl, C₁-C₅-alkoxy, C₁-C₅-alkylthio, C₁-C₅-perfluoroalkyl, halogen, hydroxy, —O—(CH₂)ₙ—O— and —O—(CH₂)ₙ—CH₂— are especially preferred. In particular, compounds whose substituents of A are selected from the group of C₁-C₅-alkyl, C₁-C₅-alkoxy, C₁-C₅-alkylthio, C₁-C₅-perfluoroalkyl, halogen and hydroxy are a subject of the invention.

Compounds whose substituents of A are selected from the group of —O—(CH₂)—O—, —O—(CH₂)ₙ—CH₂—, —O—CH═CH— and —CH₂)ₙ₊₂—, preferably —O—(CH₂)ₙ—O— and —O—(CH₂)ₙ—CH₂—, are another subject of the invention.

Compounds of formula I, in which A means a phenyl radical and whose substituents are selected from the group of hydroxy, C₁-C₅-alkoxy and halogen, are another subject of the invention.

The following definitions and substitution patterns in ring A are a special subject of the invention: 2,5-disubstituted phenyl derivatives and 2,4-disubstituted phenyl derivatives.

For radical B, the unsubstituted methylene group and the carbonyl group are preferred.

Compounds of formula I according to claim 1, in which B stands for a methylene group that is optionally substituted by a methyl or ethyl group, are a special subject of the invention.

Compounds according to claim 1, in which Q means a benzothiazolyl, quinazolinyl, quinoxalinyl, cinnolinyl, phthalazinyl, 1,7- or 1,8-naphthyridinyl, indazolyl, dihydroindolonyl, dihydroisoindolonyl, benzimidazolyl or indolyl group that is linked via any position, are a subject of the invention.

Compounds of formula I in which Q means a benzothiazolyl, quinazolinyl, quinoxalinyl, cinnolinyl, indazolyl, phthalazinyl or a 1,7- or 1,8-naphthyridinyl group that is linked via an position are another subject of the invention.

Preferred radicals Q are quinazoline, benzothiazole, naphthyridine, indazole, indolone, benzimidazole, and isoindolone. Especially preferred are quinazoline, indazole and benzimidazole.

Radical Q can be linked via any ring-carbon atom to the (NH)-group of the chain. 5- and 8-positions are preferred for the quinazoline ring, the quinoxaline ring, the cinnoline ring and the phthalazine ring; 3- and 5-positions are preferred for the naphthyridine ring, and 7- and 4-positions are preferred for the dihydroindolone, dihydroisoindolone, benzimidazole, indazole, indole and the benzothiazole ring.

For the purposes of this invention, the expression that Q “can be linked via any ring-carbon atom or any position” means any possible chemical linkage between one of the carbon atoms of the heterocyclic compound Q and the NH group of the compound of formula I.

Q can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro or NR⁴R⁵, whereby R⁴ and R⁵, independently of one another, can be hydrogen, C₁-C₅-alkyl or (CO)—C₁-C₅-alkyl.

The C₁-C₃-alkyl group, the C₁-C₅-alkoxy group, the hydroxy group, the C₁-C₅-perfluoroalkyl group and halogen atoms are preferred. The C₁-C₃-alkyl group, the hydroxy group and halogen atoms are especially preferred.

Anther subject of this invention follows from the meanings for A, R¹, R², R³, R⁴, R⁵, B and Q, disclosed in the Examples, and all possible combinations thereof.

The compounds of general formula I according to the invention can be present as different stereoisomers because of the presence of asymmetry centers. Both the racemates and the separately present stereoisomers are part of the subject of this invention.

With respect to their active strength, the separately present stereoisomers, i.e., (+)-enantiomers and (−)-enantiomers, are a special subject of this invention.

In the case that the compounds of general formula I are present as salts, the latter can be in the form of, for example, hydrochloride, sulfate, nitrate, phosphate, pivalate, maleate, fumarate, tartrate, benzoate, mesylate, citrate or succinate, which can be obtained according to the methods that are known to one skilled in the art.

The process for the production of the compounds of WO98/54159, WO00/32584 and WO02/10143 can also be used for the production of the compounds according to the invention. For the linkage of the benzothiazole, quinazoline, quinoxaline, cinnoline, indazole, phthalazine, 1,7- and 1,8-naphthyridine, dihydroindolone, dihydroisoindolone, benzimidazole or indole groups that are characteristic of the compounds according to the invention, the following process steps can be performed:

A1)

for B=CO

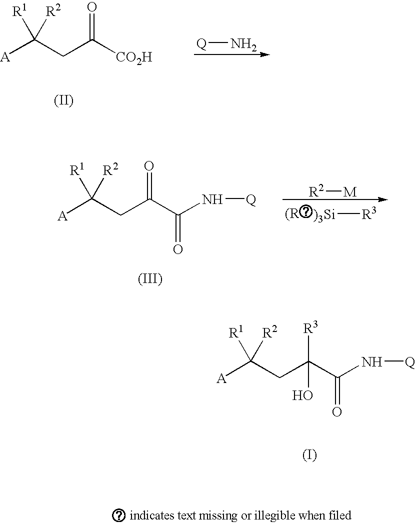

A □-keto acid of general formula (II), in which A, R¹ and R² have the meanings that are indicated for formula (I), is converted with an aminobenzothiazole, aminoquinazoline, aminoquinoxaline, aminocinnoline, or aminophthalazine derivatives (Q-NH₂) into □-ketoamide (III), whereby A, R¹ and R² have the above-indicated meanings, in the way that is known to one skilled in the art. For example, □-ketoamide (III) is obtained with use of dehydrating coupling reagents, as they are known from peptide chemistry, e.g., dicyclohexylcarbodiimide or 1-(3-dimethylaminopropyl)-3-ethylcarbodiimides, or by upstream conversion of the acid into an acid chloride, e.g., with thionyl chloride or POCl₃ and subsequent reaction with Q-NH₂.

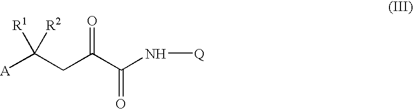

Compound (III) is reacted either with an alkyl metal compound R³-M, in which R³ has the above-indicated meanings and M stands for an alkali metal (lithium, sodium or potassium) or MgX or ZnX with X=halogen (chlorine, bromine, or iodine), or by reaction with compound (IV),
(R⁶)₃Si—R³ (IV)
whereby R³ has the above-indicated meaning, and R⁶ refers to a C₁-C₅-alkyl group, and the three R⁶ groups must not be the same, in the presence of a catalyst, e.g., fluoride salts or bases, such as, for example, alkali carbonates (J. Am. Chem. Soc. 1989, 111, 393), to form title compound (I).
A2)
for B=CO 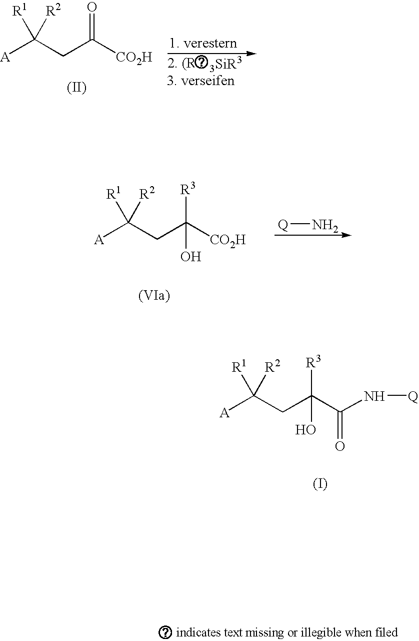
[Key: verestern=esterification
   verseifen=saponification]

As an alternative, □-keto acids (II) can also be esterified to compounds (V), 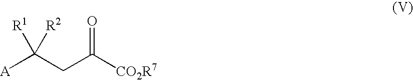
in which A, R¹ and R² are defined as described above, and R⁷ is C₁-C₄-alkyl, according to commonly used methods, e.g., with thionyl chloride in methanol or ethanol or with methyl iodide and alkali carbonate, and can be reacted analogously to reaction sequence A1) from (III) to (I) with alkyl compounds of formula R³-M, in which R³ has the above-indicated meanings, and M stands for an alkali metal (lithium, sodium or potassium) or MgX or ZnX with X=halogen (chlorine, bromine, or iodine), or with (R⁶)₃Si—R³ to form compound (VI).

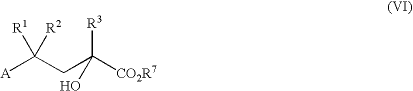

The ester (VI) is saponified under standard conditions, for example aqueous alkali hydroxide solution, to acid (VIa; R⁷=H).

The acid (VIa) is reacted for coupling with an aminoquinazoline, aminoquinoxaline, aminocinnoline, aminoindazole, aminophthalazine, aminonaphthyridine, aminobenzothiazole, aminodihydroindolone, aminodihydroisoindolone, aminobenzimidazole or aminoindole with use of a conventional activating reagent, e.g., thionyl chloride, optionally in the presence of a catalyst such as dimethylaminopyridine, to form title compound (I).

B)

for B=a Methylene Group that is Optionally Substituted by Methyl or Ethyl

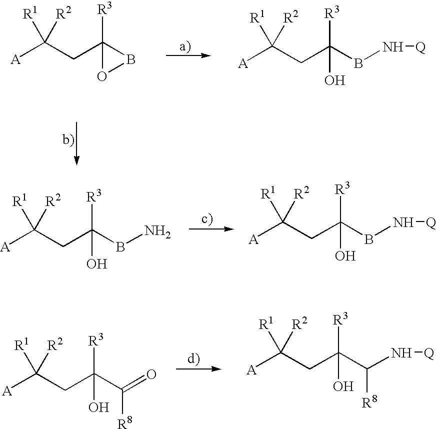

a)

A compound of general formula (VII) or (VIII), 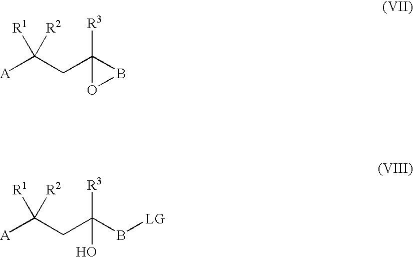
in which A, B and R¹, R² and R³ have the above-indicated meanings, and LG means any leaving group such as halide or sulfonate, is reacted with a compound of general formula (IX) or (X)
Q-NH—R⁹ (IX)
Q-N═C═O (X)
in which R⁹ means a hydrogen atom, a C₁-C₅-acyl group or alkoxy or aryloxycarbonyl group, and Q has the above-indicated meaning, whereby radical R⁹ is cleaved off, or an intermediately formed oxazolidinone (cf., e.g., S. J. Brickner, D. K. Hutchinson, M. R. Barbachyn, P. R. Manninen, D. A. Ulanowicz, S. A. Garmon, K. C. Grega, S. K. Hendges, D. S. Toops, C. W. Ford, G. E. Zurenko J. Med. Chem. 1996, 39, 673) is cleaved with, for example, aqueous alkali hydroxides to produce title compound (I).

b)

Another method consists in reacting compounds of formula (VII) or (VIII) with nitrogen nucleophiles, for example azide salts or ammonia, whereby in the first case, a reduction step follows in the way that is known to one skilled in the art, e.g., with complex hydride reagents, such as lithium aluminum hydride, or by a transition metal-catalyzed hydrogenolysis to produce compounds of formula (XI).

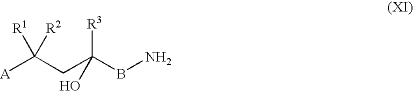

Radicals R¹-R³, A and B are equally important as indicated above.

c)

Compound (XI) can be converted under base catalysis, e.g., in the presence of tertiary amine bases or alkali carbonates or alkali hydroxides, or under transition metal catalysis, e.g., palladium catalysis (J. P. Wolfe, S. Wagaw, J.-F. Marcoux, S. L. Buchwald Acc. Chem. Res. 1998, 31, 805; J. F. Hartwig Acc. Chem. Res. 1998, 31, 852), with a halogenated quinazoline, quinoxaline, cinnoline, indazole, phthalazine, naphthyridine, benzothiazole, dihydroindolone, dihydroisoindolone, benzimidazole or indole, into title compound (I).

d)

Finally, title compound (I) can also be synthesized with Q-NH₂ by reductive amination of a compound of formula (XII), which can be obtained by means of reduction or alkylation from compound (VI) according to the methods that are known to one skilled in the art, whereby, e.g., sodium cyanoborohydride, sodium triacetoxy borohydride or hydrogen is considered as a reducing agent under palladium catalysis.

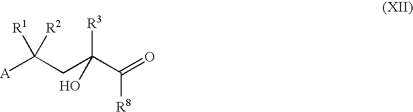

R⁸ means hydrogen, methyl or ethyl according to the substituents that are defined for the methylene group in B.

In the case that the compounds of general formula I are present as salts, this can be, for example, in the form of hydrochloride, sulfate, nitrate, phosphate, pivalate, maleate, fumarate, tartrate, benzoate, mesylate, citrate or succinate.

If the compounds according to the invention are present as racemic mixtures, they can be separated into pure, optically active forms according to the methods of racemate separation that are familiar to one skilled in the art. For example, the racemic mixtures can be separated by chromatography on an even optically active carrier material (CHIRALPAK AD®) into the pure isomers. It is also possible to esterify the free hydroxy group in a racemic compound of general formula I with an optically active acid. The diastereoisomeric esters that are obtained can be separated by fractionated crystallization or by chromatography. The separated esters are then saponified in each case to the optically pure isomers. As an optically active acid, for example, mandelic acid, camphorsulfonic acid or tartaric acid can be used.

The binding of the substances to the glucocorticoid receptor (GR) and other steroid hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses, which code for the GR, are used for the binding studies. In comparison to reference substance [³H]-dexamethasone, the substances show a high to very high affinity to GR.

Moreover, the compounds of formula (I) substituted by quinazolines, quinoxalines, cinnolines, indazoles, phthalazines, naphthyridines, benzothiazoles, dihydroindolones, dihydroisoindolones, benzimidazoles and indoles that are described here show a high selectivity for the glucocorticoid receptor. Example 2 thus shows, e.g., the following profile: IC₅₀(GR)=1.8 nmol; IC₅₀(MR), IC₅₀(PR), IC₅₀(AR)>1 □mol, and the compound of Example 52: IC₅₀(GR)=10 nmol; IC₅₀(MR), IC₅₀(PR), IC₅₀(AR)>1 □mol.

As an essential, molecular mechanism for the anti-inflammatory action of glucocorticoids, the GR-mediated inhibition of the transcription of cytokines, adhesion molecules, enzymes and other pro-inflammatory factors is considered. This inhibition is produced by an interaction of the GR with other transcription factors, e.g., AP-1 and NF-kappa-B (for a survey, see Cato, A. C. B. and Wade, E., BioEssays 18, 371-378, 1996).

The compounds of general formula I according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits.

The anti-inflammatory action of the compounds of general formula I was tested in the animal experiment by tests in the croton oil-induced inflammation in rats and mice (J. Exp. Med. (1995), 182, 99-108). To this end, croton oil in ethanolic solution was applied topically to the animals' ears. The test substances were also applied topically or systemically at the same time or two hours before the croton oil. After 16-24 hours, the ear weight was measured as a yardstick for inflammatory edema, the peroxidase activity as a yardstick for the invasions of granulocytes, and the elastase activity as a yardstick for the invasion of neutrophilic granulocytes. In this test, the compounds of general formula I inhibit the three above-mentioned inflammation parameters both after topical administration and after systemic administration.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Bases, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula I induce little or no tyrosinamino transferase.

Because of their anti-inflammatory and, in addition, anti-allergic, immunosuppressive and antiproliferative action, the compounds of general formula I according to the invention can be used as medications for treatment or prophylaxis of the following pathologic conditions in mammals and humans: In this case, the term “DISEASE” stands for the following indications:

(i) Lung Diseases that are Accompanied by Inflammatory, Allergic and/or Proliferative Processes:
Chronic, obstructive lung diseases of any origin, primarily bronchial asthma
Bronchitis of different origins
All forms of restrictive lung diseases, primarily allergic alveolitis,
All forms of pulmonary edema, primarily toxic pulmonary edema
Sarcoidoses and granulomatoses, especially Boeck's disease

(ii) Rheumatic Diseases/Autoimmune Diseases/Joint Diseases that are Accompanied by Inflammatory, Allergic and/or Proliferative Processes:
All forms of rheumatic diseases, especially rheumatoid arthritis, acute rheumatic fever, polymyalgia rheumatica
Reactive arthritis
Inflammatory soft-tissue diseases of other origins
Arthritic symptoms in the case of degenerative joint diseases (arthroses)
Traumatic arthritides
Collagenoses of any origin, e.g., systemic lupus erythematodes, sclerodermia, polymyositis, dermatomyositis, Sjögren's syndrome, Still's syndrome, Felty's syndrome

(iii) Allergies that are Accompanied by Inflammatory and/or Proliferative Processes:
All forms of allergic reactions, e.g., Quincke's edema, hay fever, insect bites, allergic reactions to pharmaceutical agents, blood derivatives, contrast media, etc., anaphylactic shock, urticaria, contact dermatitis

(iv) Vascular inflammations (vasculitides)
Panarteritis nodosa, temporal arteritis, erythema nodosum

(v) Dermatological Diseases that are Accompanied by Inflammatory, Allergic and/or Proliferative Processes:
Atopic dermatitis (primarily in children)
Psoriasis
Pityriasis rubra pilaris
Erythematous diseases, triggered by different noxae, e.g., radiation, chemicals, bums, etc.
Bullous dermatoses
Diseases of the lichenoid group,
Pruritis (e.g., of allergic origin)
Seborrheal eczema
Rosacea
Pemphigus vulgaris
Erythema exudativum multiforme
Balanitis
Vulvitis
Hair loss such as alopecia areata
Cutaneous T-cell lymphoma

(vi) Kidney Diseases that are Accompanied by Inflammatory, Allergic and/or Proliferative Processes:
Nephrotic syndrome
All nephritides

(vii) Liver Diseases that are Accompanied by Inflammatory, Allergic and/or Proliferative Processes:
Acute liver cell decomposition
Acute hepatitis of different origins, e.g., viral, toxic, pharmaceutical agent-induced
Chronic aggressive hepatitis and/or chronic intermittent hepatitis

(viii) Gastrointestinal Diseases that are Accompanied by Inflammatory, Allergic and/or Proliferative Processes:
Regional enteritis (Crohn's disease)
Colitis ulcerosa
Gastritis
Reflux esophagitis
Ulcerative colitis of other origins, e.g., native sprue

(ix) Proctologic Diseases that are Accompanied by Inflammatory, Allergic and/or Proliferative Processes:
Anal eczema
Fissures
Hemorrhoids
Idiopathic proctitis

(x) Eye Diseases that are Accompanied by Inflammatory, Allergic and/or Proliferative Processes:
Allergic keratitis, uveitis, iritis
Conjunctivitis
Blepharitis
Optic neuritis
Chorioiditis
Sympathetic ophthalmia

(xi) Diseases of the Ear-Nose-Throat Area that are Accompanied by Inflammatory, Allergic and/or Proliferative Processes:
Allergic rhinitis, hay fever
Otitis externa, e.g., caused by contact dermatitis, infection, etc.
Otitis media

(xii) Neurological Diseases that are Accompanied by Inflammatory, Allergic and/or Proliferative Processes:
Cerebral edema, primarily tumor-induced cerebral edema
Multiple sclerosis
Acute encephalomyelitis
Meningitis
Various forms of convulsions, e.g., infantile nodding spasms

(xiii) Blood Diseases that are Accompanied by Inflammatory, Allergic and/or Proliferative Processes:
Acquired hemolytic anemia
Idiopathic thrombocytopenia

(xiv) Tumor Diseases that are Accompanied by Inflammatory, Allergic and/or Proliferative Processes:
Acute lymphatic leukemia
Malignant lymphoma
Lymphogranulomatoses
Lymphosarcoma
Extensive metastases, mainly in breast, bronchial and prostate cancers

(xv) Endocrine Diseases that are Accompanied by Inflammatory, Allergic and/or Proliferative Processes:
Endocrine orbitopathy
Thyreotoxic crisis
De Quervain's thyroiditis
Hashimoto's thyroiditis
Basedow's disease

(xvi) Organ and Tissue Transplants, Graft-Versus-Host Disease

(xvii) Severe Shock Conditions, e.g., Anaphylactic Shock, Systemic Inflammatory Response Syndrome (SIRS)

(xviii) Substitution Therapy in:
Innate primary suprarenal insufficiency, e.g., congenital adrenogenital syndrome
Acquired primary suprarenal insufficiency, e.g., Addison's disease, autoimmune adrenalitis, meta-infective tumors, metastases, etc.
Innate secondary suprarenal insufficiency, e.g., congenital hypopituitarism
Acquired secondary suprarenal insufficiency, e.g., meta-infective tumors, etc.

(xix) Vomiting that is Accompanied by Inflammatory, Allergic and/or Proliferative Processes:

e.g., in combination with a 5-HT3 antagonist in cytostatic-agent-induced vomiting (xx) Pains of Inflammatory Origins, e.g., Lumbago.

Moreover, the compounds of general formula I according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications (i) to (xx) are described in more detail in Hatz, H. J., Glucocorticoide: Immunologische Grundlagen, Pharmakologie und Therapierichtlinien, Wissenschaftliche Verlagsgesellschaft mbH, Stuttgart, 1998.

For the therapeutic actions in the above-mentioned pathologic conditions, the suitable dose varies and depends on, for example, the active strength of the compound of general formula I, the host, the type of administration, and the type and severity of the conditions that are to be treated, as well as the use as a prophylactic agent or therapeutic agent.

In addition, the invention provides:
(i) The use of one of the compounds of general formula I according to the invention or mixture thereof for the production of a medication for treating a DISEASE;
(ii) A process for treating a DISEASE, said process comprises an administration of an amount of the compound according to the invention, whereby the amount suppresses the disease and whereby the amount of compound is given to a patient who requires such a medication;
(iii) A pharmaceutical composition for treating a DISEASE, said treatment comprises one of the compounds according to the invention or mixture thereof and at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), individual doses can be given that are significantly above the above-mentioned doses.

The formulation of the pharmaceutical preparations based on the new compounds is carried out in a way that is known in the art by the active ingredient being processed with the vehicles that are commonly used in galenicals, fillers, substances that influence decomposition, binding agents, moisturizers, lubricants, absorbents, diluents, flavoring correctives, coloring agents, etc., and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments and tinctures in corresponding pharmaceutical preparations.

For topical application, formulations in gels, ointments, fatty ointments, creams, pastes, powders, milk and tinctures are possible. The dosage of the compounds of general formula I should be 0.01%-20% in these preparations to achieve a sufficient pharmacological action.

The invention also comprises the compounds of general formula I according to the invention as therapeutic active ingredients.

In addition, the compounds of general formula I are part of the invention as therapeutic active ingredients together with pharmaceutically compatible and acceptable adjuvants and vehicles. The invention also comprises a pharmaceutical composition that contains one or more of the pharmaceutically active compounds according to the invention or mixtures thereof or a pharmaceutically compatible salt thereof or pharmaceutically compatible adjuvants and vehicles.

The examples below are used for a more detailed explanation of the invention without intending that it be limited thereto. The syntheses of important precursors, which are not disclosed within the scope of the experiments, are already prior art and can be

### EXPERIMENTS

### Example 1

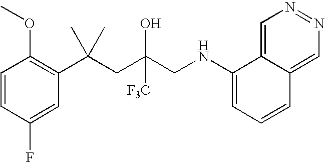

### 4-(5-Fluoro-2-methoxyphenyl)-4-methyl-1-(phthalazin-5-ylamino)-2-(trifluoromethyl)-pentan-2-ol

4-(5-Fluoro-2-methoxy-phenyl)-2-hydroxy-4-methyl-2-trifluoromethyl-pentanal

0.81 ml (8.67 mmol) of oxalyl chloride is cooled in 15 ml of dichloromethane to −60° C. and mixed with 1.6 ml (22.6 mmol) of dimethyl sulfoxide in 10 ml of dichloromethane. After 15 minutes, 1.0 g (3.22 mmol) of 4-(5-fluoro-2-methoxy-phenyl)-4-methyl-2-trifluoromethyl-pentane-1,2-diol (WO 00/32584) in 10 ml of dichloromethane is added, and the mixture is stirred for one hour at −60° C. 4.1 ml (29 mmol) of triethylamine is added, and the mixture is allowed to heat over 30 minutes to room temperature. It is poured into 50 ml of water and extracted with CH₂Cl₂. The combined organic extracts are washed with saturated NaCl solution, dried (Na₂SO₄) and concentrated by evaporation in a vacuum. After chromatography on silica gel with hexane-ethyl acetate (0-30%), 600 mg of the product is obtained.

¹H-NMR (CDCl₃); δ=1.38 (s, 3H), 1.47 (s, 3H), 2.23 (d, 1H), 3.36 (d, 1H), 3.86 (s, 3H), 6.77 (dd, 1H), 6.87 (dd, 1H), 6.91 (ddd, 1H), 9.05 (s, 1H).

200 mg (0.65 mmol) of 4-(5-fluoro-2-methoxy-phenyl)-2-hydroxy-4-methyl-2-trifluoromethyl-pentanal in 5 ml of toluene is added to 70 mg (0.48 mmol) of 5-aminophthalazine (I. A. Shaikh, F. Johnson, A. P. Grollman, J. Med. Chem. 1986, 26, 1329-1340) in 2 ml of acetic acid. The reaction solution is refluxed with water being separated off for 6 hours and refluxed on a molecular sieve (4 A) for another 4 hours. The solvent is removed in a vacuum, and acetic acid residue is eliminated by azeotropic codistillation with toluene. After chromatography on silica gel with hexane-ethyl acetate (0-70%), 40 mg of 4-(5-fluoro-2-methoxyphenyl)-4-methyl-1-(phthalazin-5-ylimino)-2-(trifluoromethyl)-pentan-2-ol is obtained. 20 mg of palladium on carbon is added to 10 mg of imine in 10 ml of ethyl acetate and 1 ml of triethylamine, and it is shaken for 2 hours under a hydrogen atmosphere at normal pressure. Catalyst is removed from the solution by means of filtration, and it is concentrated by evaporation. After chromatography on silica gel with hexane-ethyl acetate (0-70%), 4 mg of the desired product is obtained.

¹H-NMR (CDCl₃); δ=1.44 (s, 3H), 1.66 (s, 3H), 2.07 (d, 1H), 3.07 (d, 1H), 3.16 (d, 1H), 3.24 (d, 1H), 3.85 (s, 3H), 6.42 (d, 1H), 6.76 (m, 2H), 7.11 (dd, 1H), 7.30 (d, 1H), 7.66 (dd, 1H), 9.38 (s, 1H), 9.48 (s, 1H).

### Example 2

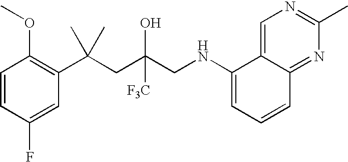

### 4-(5-Fluoro-2-methoxyphenyl)-4-methyl-1-(2-methylquinazolin-5-ylamino)-2-(trifluoromethyl)-pentan-2-ol

5-Amino-2-methylquinazoline

12.7 g (mmol) of 2-methyl-5-nitro-3H-quinazolin-4-one (M. T. Bogert, V. J. Chambers J. Org Chem. 1905, 649-658) and 37.5 g of phosphorus pentachloride are refluxed in 75 ml of phosphoryl chloride over 20 hours. After cooling, it is poured into saturated NaHCO₃ solution and extracted with ethyl acetate. The organic phase is dried, and the solvent is removed. 14 g of 4-chloro-2-methyl-5-nitroquinazoline, of which 4.5 g (20.2 mmol) in 225 ml of ethyl acetate and 22.5 ml of triethylamine are dissolved, is obtained. 2 g of palladium on carbon is added, and it is stirred while being cooled with ice for 4 hours under a hydrogen atmosphere at normal pressure. Catalyst is removed from the solution by means of filtration on Celite, whereby washing is continued with 200 ml of ethanol, and it is concentrated by evaporation. After chromatography on silica gel with ethyl acetate-ethanol (0-10%), 530 mg of the product is obtained. ¹H-NMR (CDCl₃); δ=2.87 (s, 3H), 4.52 (br., 2H), 6.77 (d, 1H), 7.33 (d, 1H), 7.65 (t, 1H), 9.40 (s, 1H).

180 mg (0.48 mmol) of 4-(5-fluoro-2-methoxyphenyl)-2-hydroxy-4-methyl-2-(trifluoromethyl)pentanal and 50 mg of 5-amino-2-methylquinazoline are concentrated to 5 ml in 20 ml of dichloroethane and 2 ml of acetic acid with continuous slow removal of the solvent over 5 hours. The residual solvent is removed in a vacuum, and acetic acid residue is eliminated by azeotropic codistillation with toluene. After chromatography on silica gel with hexane-ethyl acetate (0-70%), 58 mg of 4-(5-fluoro-2-methoxyphenyl)-4-methyl-1-(2-methylquinazolin-5-ylimino)-2-(trifluoromethyl)-pentan-2-ol is obtained. 20 mg of palladium on carbon is added to the imine in 10 ml of ethyl acetate and 1 ml of triethylamine, and it is shaken for 2 hours under a hydrogen atmosphere at normal pressure. Catalyst is removed from the solution by means of filtration, and it is concentrated by evaporation. It is taken up in 5 ml of chloroform, and 200 mg of activated manganese dioxide is added and stirred for 30 minutes. It is filtered on Celite and concentrated by evaporation in a vacuum. After chromatography on silica gel with hexane-ethyl acetate (0-70%), 22 mg of the product is obtained. ¹H-NMR (CDCl₃); δ=1.47 (s, 3H), 1.56 (s, 3H), 2.38 (d, 1H), 2.77 (d, 1H), 2.83 (s, 3H), 3.16 (dd, 1H), 3.33 (dd, 1H), 3.85 (s, 3H), 4.70 (br., 1H), 6.05 (d, 1H), 6.77 (dd, 1H), 6.88 (ddd, 1H), 7.09 (dd, 1H), 7.24 (d, 1H), 7.56 (t, 1H), 9.16 (s, 1H).

### Example 3

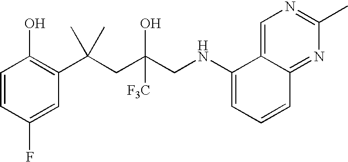

### 4-(5-Fluoro-2-hydroxyphenyl)-4-methyl-1-(2-methylquinazolin-5-ylamino)-2-(trifluoromethyl)-pentan-2-ol

103 mg (0.23 mmol) of 4-(5-fluoro-2-methoxyphenyl)-4-methyl-1-(2-methylquinazolin-5-ylamino)-2-(trifluoromethyl)-pentan-2-ol in 10 ml of CH₂Cl₂ is mixed at 0° C. with 5 ml of 1 M boron tribromide-CH₂Cl₂ solution. After 10 hours, another 5 ml of 1 M boron tribromide-CH₂Cl₂ solution is added, and at room temperature, the batch is poured into saturated NaHCO₃ after 72 hours, stirred for 20 minutes and extracted with CH₂Cl₂. The combined organic extracts are washed with water, dried (Na₂SO₄) and concentrated by evaporation in a vacuum. Chromatography with hexane-2-propanol (0-20%) on silica gel yields 80 mg of the product.

¹H-NMR (CDCl₃); δ=1.51 (s, 3H), 1.58 (s, 3H), 2.37 (d, 1H), 2.81 (s, 3H), 2.91 (d, 1H), 3.25 (dd, 1H), 3.43 (dd, 1H), 5.05 (br., 1H), 6.20 (d, 1H), 6.54 (dd, 1H), 6.69 (m, 1H), 7.05 (dd, 1H), 7.23 (d, 1H), 7.59 (d, 1H), 7.58 (d, 1H), 8.32 (d, 1H), 8.68 (d, 1H).

### Example 4

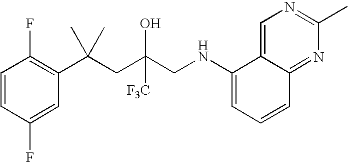

### 4-(2,5-Difluorophenyl)-4-methyl-1-(2-methylquinazolin-5-ylamino)-2-(trifluoromethyl)-pentan-2-ol

4-(2,5-Difluorophenyl)-2-hydroxy-4-methyl-2-trifluoromethyl-pentanal

5.4 g (15.5 mmol) of 4-(2,5-difluorophenyl)-2-hydroxy-4-methyl-2-trifluoromethyl-valeric acid ethyl ester (WO 02/10143) is dissolved at 0° C. in diethyl ether and mixed within 20 minutes with 1.76 g (46.5 mmol) of lithium aluminum hydride. It is allowed to stir at room temperature for 4 hours, and enough saturated NaHCO₃ solution is carefully added until no more gas generation is observed. The mixture is diluted with ethyl acetate, stirred for another 15 minutes, and then the precipitate that is formed is filtered off. It is concentrated by evaporation and chromatographed on silica gel with hexane/ethyl acetate (50%). 2.45 g of 2,5-difluorophenyl)-4-methyl-2-trifluoromethyl-pentane-1,2-diol is obtained as a pale yellowish crystallizing oil. 800 mg (2.8 mmol) of 4-(2,5-difluorophenyl)-4-methyl-2-trifluoromethyl-pentane-1,2-diol is introduced into 20 ml of dichloromethane, and at 0° C., 9.5 ml of DMSO and 1.95 ml of triethylamine are added. The solution is slowly mixed with 1.34 g (8.4 mmol) of SO₃-pyridine complex, and it is stirred for 2 hours at 0° C. The mixture is dispersed between saturated ammonium chloride solution and MTBE, the phases are separated, and the aqueous phase is extracted with MTBE. The combined organic phases are washed with water and saturated NaCl solution and dried with NaSO₄. It is concentrated by evaporation and chromatographed on silica gel with hexane/ethyl acetate (30%). 710 mg of the desired product is obtained. ¹H-NMR (CDCl₃): δ=1.41 (s, 3H), 1.48 (s, 3H), 2.39 (d, 2H), 3.02 (d, 1H), 3.61 (s, 1H), 6.84-7.18 (m, 3H), 9.23 (s, 1H).

240 mg (0.84 mmol) of 4-(2,5-difluoro-phenyl)-2-hydroxy-4-methyl-2-trifluoromethyl-pentanal and 200 mg (1.26 mmol) of 5-amino-2-methyl-quinazoline are reacted first analogously to Example 2. After chromatography on silica gel with hexane-ethyl acetate (0-70%), 80 mg of 4-(2,5-difluorophenyl)-4-methyl-1-(2-methylquinazolin-5-ylimino)-2-(trifluoromethyl)-pentan-2-ol is obtained and is taken up again in ethyl acetate/ethanol 1:1 and hydrogenated with 10 mg of palladium catalyst (10% on activated carbon) under hydrogen atmosphere (1 atm). After 5 hours at room temperature, the catalyst is suctioned off, and the filtrate is concentrated by evaporation. The residue is taken up again in chloroform and reacted with manganese dioxide analogously to Example 2. After chromatographic purification, 15 mg of the desired product is obtained as a reddish-brown film. MS (ESI): 440 (M+H); ¹H-NMR (CDCl₃): δ=1.48 (s, 3H), 1.62 (s, 3H), 2.29 (d, 1H), 2.61 (d, 1H), 2.79 (s, 3H), 3.19-3.35 (m, 2H), 3.61 (s, 1H), 4.69-4.73 (m, 1H), 6.00 (d, 1H), 6.83-6.91 (m, 2H), 7.08-7.14 (m, 1H), 7.23 (d, 1H), 7.52 (dd, 1H), 9.14 (d, 1H).

### Example 5

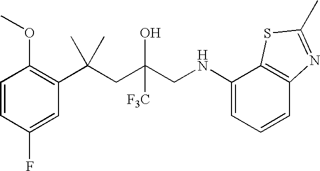

### 4-(5-Fluoro-2-methoxyphenyl)-4-methyl-1-(2-methylbenzothiazol-7-ylamino)-2-(trifluoromethyl)-pentan-2-ol

200 mg (0.65 mmol) of 4-(5-fluoro-2-methoxy-phenyl)-2-hydroxy-4-methyl-2-trifluoromethyl-pentanal and 126 mg (0.77 mmol) of 7-amino-2-methylbenzothiazole (Libeer et al. Bull. Soc. Chim. Belg.; 1971; 80; 43-47) are heated in 8 ml of acetic acid over 5 hours to 125° C. After cooling to room temperature, it is mixed with 214 mg (1.01 mmol) of sodium triacetoxy borohydride and allowed to stir for 16 hours. After another 100 mg (0.47 mmol) of sodium triacetoxy borohydride is added, and after 2 hours of stirring, toluene is added, and it is concentrated by evaporation in a vacuum. The residue is taken up in ethyl acetate, the organic phase is washed with saturated sodium bicarbonate and saturated sodium chloride solution, and it is dried on sodium sulfate. After chromatography on silica gel with hexane-ethyl acetate (0-50%), 221 mg of the product is obtained. ¹H-NMR (CDCl₃); δ=1.45 (s, 3H), 1.58 (s, 3H), 2.25 (d, 1H), 2.78 (d, 1H), 2.82 (s, 3H), 3.14 (s, 1H), 3.16 (dd, 1H), 3.28 (dd, 1H), 3.48 (dd, 1H), 3.84 (s, 3H), 4.23 (d, 1H), 5.97 (d, 1H), 6.82 (dd, 1H), 6.96 (ddd, 1H), 7.15 (dd, 1H), 7.21 (t, 1H), 7.42 (d, 1H).

### Example 6

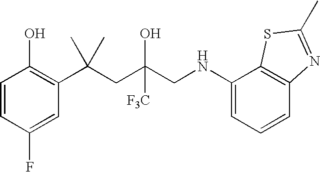

### 4-(5-Fluoro-2-hydroxyphenyl)-4-methyl-1-(2-methylbenzothiazol-7-ylamino)-2-(trifluoromethyl)pentan-2-ol

Analogously to Example 3, 150 mg (0.13 mmol) of 4-(5-fluoro-2-methoxyphenyl)-4-methyl-1-(2-methylbenzothiazol-7-ylamino)-2-(trifluoromethyl)-pentan-2-ol in 15 ml of CH₂Cl₂ is reacted with 6.8 ml of 1 M boron tribromide-CH₂Cl₂ solution. After chromatography on silica gel with hexane-ethyl acetate (0-70%), 102 mg of the product is obtained. ¹H-NMR (CDCl₃); δ=1.50 (s, 3H), 1.59 (s, 3H), 2.31 (d, 1H), 2.79 (d, 1H), 2.80 (s, 3H), 3.27 (m, 2H), 3.40 (dd, 1H), 3.54 (dd, 1H), 6.02 (d, 1H), 6.11 (br., 1H), 6.65 (dd, 1H), 6.82 (ddd, 1H), 7.12 (dd, 1H), 7.18 (t, 1H), 7.40 (d, 1H).

### Example 7

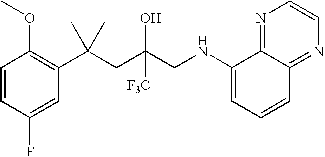

### 1-(Quinoxalin-5-ylamino)-4-(5-fluoro-2-methoxyphenyl)-4-methyl-2-(trifluoromethyl)-pentan-2-ol

140 mg (0.46 mmol) of 4-(5-fluoro-2-methoxy-phenyl)-2-hydroxy-4-methyl-2-trifluoromethyl-pentanal, dissolved in 5 ml of dichloroethane, is added to 80 mg (0.55 mmol) of 5-aminoquinoxaline (J. Salon, V. Milata, N. Pronayova, J. Lesko Monatsh. Chem. 2000, 131, 293-299) in 2 ml of acetic acid. The reaction solution is refluxed for 5 hours on a molecular sieve (4 A). The mixture is dispersed between water and dichloromethane and extracted (CH₂Cl₂). The combined organic phases are washed (saturated NaCl solution), dried (Na₂SO₄) and concentrated by evaporation. After chromatographic purification on silica gel with hexane/ethyl acetate (0-50%), 82 mg of 1-(quinoxalin-5-ylimino)-4-(5-fluoro-2-methoxyphenyl)-4-methyl-2-(trifluoromethyl)-pentan-2-ol, which is taken up in 3 ml of methanol and mixed with 100 μl of acetic acid and 10 mg (0.26 mmol) of NaBH₄, is obtained. The reaction mixture is stirred for 2 days at room temperature, and here, another 10 mg of NaBH₄ is added twice in each case. The mixture is dispersed between water and dichloromethane and extracted (CH₂Cl₂). The combined organic phases are washed (saturated NaCl solution), dried (Na₂SO₄) and concentrated by evaporation. The crude product is purified by chromatography on silica gel with hexane/ethyl acetate (10-50%). 40 mg of the desired product, which can be recrystallized from hexane/diethyl ether, is obtained. MS (ESI): 438 (M+H); ¹H-NMR (CDCl₃): δ=1.46 (s, 3H), 1.61 (s, 3H), 2.26 (d, 1H), 2.80 (d, 1H), 2.99 (s, 1H), 3.22-3.49 (m, 3H), 3.85 (s, 3H), 6.07 (d, 1H), 6.81 (dd, 1H), 6.91-6.99 (m, 1H), 7.19 (dd, 1H), 7.36 (dd, 1H), 7.46 (d, 1H), 8.61 (d, 1H), 8.80 (d, 1H).

### Example 8

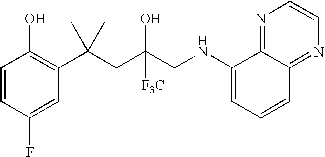

### 1-(Quinoxalin-5-ylamino)-4-(5-fluoro-2-hydroxyphenyl)-4-methyl-2-(trifluoromethyl)-pentan-2-ol

Analogously to Example 3, 30 mg (68 μmol) of 1-(quinoxalin-5-ylamino)-4-(5-fluoro-2-methoxyphenyl)-4-methyl-2-(trifluoromethyl)-pentan-2-ol, dissolved in 3 ml of dichloromethane, is reacted with 3 ml of boron tribromide solution (1 M in CH₂Cl₂) and stirred at room temperature for 24 hours. The mixture is dispersed between ethyl acetate and saturated NaHCO₃ solution and extracted with ethyl acetate. The combined organic phases are washed (saturated NaCl solution), dried (Na₂SO₄) and concentrated by evaporation. The crude product is purified by chromatography on silica gel with hexane/ethyl acetate (20%). 15 mg of the desired product is obtained. MS (ESI): 424 (M+H); ¹H-NMR (CDCl₃): δ=1.46 (s, 3H), 1.53 (s, 3H), 2.28 (d, 1H), 2.58 (d, 1H), 2.97 (br, 1H), 3.30-3.56 (m, 4H), 6.18 (d, 1H), 6.56 (dd, 1H), 6.76-6.83 (m, 1H), 7.15 (dd, 1H), 7.36 (d, 1H), 7.46 (d, 1H), 8.65 (d, 1H), 8.83 (d, 1H).

### Example 9

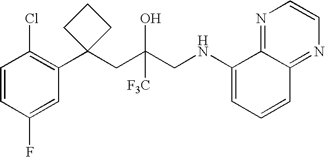

### □-[(Quinoxalin-5-ylamino)methyl]-1-(2-chloro-5-fluorophenyl)-□-(trifluoromethyl) cyclobutane ethanol

1-(2-Chloro-5-fluorophenyl)-□-hydroxy-□-(trifluoromethyl)cyclobutane propanal

3.1 g (8.7 mmol) of 1-(2-chloro-5-fluorophenyl)-□-hydroxy-□-(trifluoromethyl) cyclobutanepropionic acid ethyl ester (WO 02/10143) is reacted analogously to Example 4 with 990 mg (26.1 mmol) of lithium aluminum hydride. 1.80 g of 1-(2-chloro-5-fluorophenyl)-□-(hydroxy)-□-(trifluoromethyl)cyclobutane propanol is obtained as a pale yellowish oil. 493 μl (2.56 mmol) of oxalyl chloride is introduced into 20 ml of dichloromethane. At −75° C., 802 μl (11.3 mmol) of DMSO is added in drops, and after 15 minutes of stirring, a solution of 800 mg (2.56 mmol) of 1-[(chloro-5-fluorophenyl)-□-(hydroxy)-□-(trifluoromethyl)cyclobutane propanol in 10 ml of dichloromethane is added in drops. After another 15 minutes, 2.20 ml (15.8 mmol) of triethylamine is added in drops and stirred for another 30 minutes at −60° C. and for 30 minutes at 0° C. The reaction is completed by adding water, the phases are separated and extracted with dichloromethane. The combined organic phases are washed with water and saturated NaCl solution and dried with NaSO₄. It is concentrated by evaporation and chromatographed on silica gel with hexane/ethyl acetate (30%). 810 mg of the desired product is obtained. MS (CI): 342 (M+NH₄); ¹H-NMR (CDCl₃): δ=1.74-1.92 (m, 1H), 2.00-2.70 (m, 5H), 2.86 (d, 1H), 3.19 (d, 1H), 3.52 (s, 1H), 6.79-6.93 (m, 1H), 7.10-7.24 (m, 2H), 8.94 (s, 1H).

200 mg (0.64 mmol) of 1-[2-chloro-5-fluorophenyl)-□-hydroxy-□-(trifluoromethyl)cyclobutane propanal in 2 ml of toluene is added to 325 mg (0.96 mmol) of 5-aminoquinoxaline (J. Salon, V. Milata, N. Pronayova, J. Lesko Monatsh. Chem. 2000, 131, 293-299) in 3 ml of acetic acid, and it is stirred for 24 hours at room temperature. The solution is dispersed between toluene and water, the aqueous phase is extracted with toluene, the combined organic phases are washed with saturated NaCl solution, dried (Na₂SO₄), and the solvent is removed. The crude □-[(quinoxalin-5-ylimino)methyl]-1-(2-chloro-5-fluorophenyl)-□-(trifluoromethyl)cyclobutane ethanol is taken up in methanol/acetic acid 1:1 and mixed with 100 mg (2.66 mmol) of NaBH₄. After 6 hours of stirring at room temperature, the reaction is brought to a halt by adding saturated NH₄Cl solution, and the mixture is diluted with dichloromethane. After extraction with dichloromethane, the combined organic phases are washed (saturated NaCl solution), dried (Na₂SO₄), and the solvent is removed. 280 mg of product is obtained as a dark red resin, which can be crystallized from hexane/diethyl ether. MS (ESI): 454 (M+H).

### Example 10

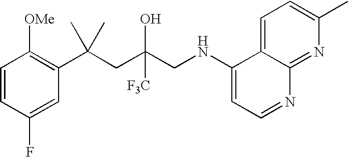

### 4-(5-Fluoro-2-methoxyphenyl)-4-methyl-1-(2-methyl-1,8-naphthyridin-5-ylamino)-2-(trifluoromethyl)-pentan-2-ol

1-Amino-4-(5-fluoro-2-methoxyphenyl)-4-methyl-2-(trifluoromethyl)propan-2-ol

1.0 g (3.4 mmol) of 2-[2-(5-fluoro-2-methoxyphenyl-2-methylpropyl]-2-(trifluoromethyl)oxirane (WO 00/32584) in 68 ml of THF is refluxed with 1.1 g of sodium azide and 180 mg of ammonium chloride in 14 ml of water and 26 ml of ethanol for 6 hours. The batch is concentrated by evaporation, diluted with ether, washed with water, dried (Na₂SO₄) and concentrated by evaporation. Chromatography on silica gel with hexane-ethyl acetate (0-15%) yields 950 mg of 1-azido-4-(5-fluoro-2-methoxyphenyl)-4-methyl-2-(trifluoromethyl)propan-2-ol. The latter is dissolved in 29 ml of THF and mixed at 0° C. in portions with 270 mg of lithium aluminum hydride. After 1 hour, the batch is treated with ethyl acetate and water and filtered on Celite. The ethyl acetate phase is dried (Na₂SO₄) and concentrated by evaporation in a vacuum. 920 mg of amine is obtained.

¹H-NMR (CDCl₃): δ=1.4 (s, 3H), 1.5 (s, 3H), 2.15 (d, 1H), 2.45 (d, 1H), 2.55 (d, 1H), 2.75 (d, 1H), 2.80 (m), 3.8 (s, 3H), 6.8 (dd, 1H), 6.9 (td, 1H), 7.05 (dd, 1H)

202 mg (1.13 mmol) of 5-chloro-2-methyl-1,8-naphthyridine (E. V. Brown, J. Org. Chem 1965, 1607-1609) is added to 350 mg (1.13 mmol) of 1-amino-4-(5-fluoro-2-methoxyphenyl)-4-methyl-2-(trifluoromethyl)-pentan-2-ol and 128 mg (1.13 mmol) of DABCO. It is heated for 1.5 hours to 150° C. After chromatography of the cooled melts on silica gel with dichloromethane/methanol (0-10%), 385 mg of the desired product is obtained. ¹H-NMR (CDCl₃); δ=1.46 (s, 3H), 1.58 (s, 3H), 2.45 (d, 1H), 2.68 (s, 3H), 2.72 (d, 1H), 3.20 (d, 1H), 3.38 (d, 1H), 3.83 (s, 3H), 5.86 (d, 1H), 6.77 (dd, 1H), 6.92 (ddd, 1H), 7.08 (dd, 1H), 7.11 (d, 1H), 7.71 (d, 1H), 8.50 (d, 1H).

### Example 11

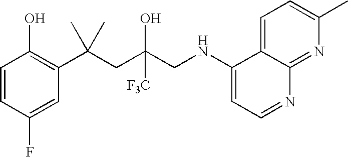

### 4-(5-Fluoro-2-hydroxyphenyl)-4-methyl-1-(2-methyl-1,8-naphthyridin-5-ylamino)-2-(trifluoromethyl)-pentan-2-ol

Analogously to Example 3, mg (0.13 mmol) of 4-(5-fluoro-2-methoxyphenyl)-4-methyl-1-(2-methyl-1,8-naphthyridin-5-ylamino)-2-(trifluoromethyl)-pentan-2-ol in 15 ml of CH₂Cl₂ is reacted with ml of 1 M boron tribromide-CH₂Cl₂ solution. After chromatography on silica gel with hexane-ethyl acetate (0-70%), 102 mg of the product is obtained. ¹H-NMR (CDCl₃); δ=1.50 (s, 3H), 1.59 (s, 3H), 2.31 (d, 1H), 2.79 (d, 1H), 2.80 (s, 3H), 3.27 (m, 2H), 3.40 (dd, 1H), 3.54 (dd, 1H), 6.02 (d, 1H), 6.11 (br., 1H), 6.65 (dd, 1H), 6.82 (ddd, 1H), 7.12 (dd, 1H), 7.18 (t, 1H), 7. (d, 1H).

### Example 12

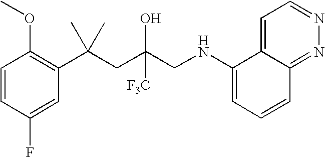

### 1-(Cinnolin-5-ylamino)-4-(5-fluoro-2-hydroxyphenyl)-4-methyl-2-(trifluoromethyl)-pentan-2-ol

240 mg (0.78 mmol) of 4-(5-fluoro-2-methoxy-phenyl)-2-hydroxy-4-methyl-2-trifluoromethyl-pentanal and 170 mg (1.17 mmol) of 5-aminocinnoline (J. R. Elkins, E. V. Brown J. Heterocycl. Chem. 1968, 639-646) are dissolved in 10 ml of dichloroethane. 1 ml of acetic acid and 30 mg of powdered molecular sieve (4 A) are added and refluxed for 6 hours on a molecular sieve (4 A). The reaction mixture is dispersed between dichloromethane and water, the phases are separated, the aqueous phase is extracted with CH₂Cl₂, the combined organic phases are washed with saturated NaHCO₃ solution and saturated NaCl solution and dried with Na₂SO₄. It is concentrated by evaporation and chromatographed on silica gel with hexane/ethyl acetate (20 to 50%). 70 mg of 1-(cinnolin-5-ylimino)-4-(5-fluoro-2-hydroxyphenyl)-4-methyl-2-(trifluoromethyl)-pentan-2-ol is obtained, of which 30 mg is taken up in THF and mixed with 10 mg (0.16 mmol) of sodium cyanoborohydride and 100 μl of acetic acid. After 6 hours of stirring at room temperature, it is dispersed between water and CH₂Cl₂, and the phases are separated. The aqueous phase is extracted with CH₂Cl₂, the combined organic phases are washed with saturated NaCl solution and dried with Na₂SO₄. It is concentrated by evaporation, the residue is taken up again in chloroform, a spatula tip full of activated manganese dioxide is added, and it is stirred for 2 hours at room temperature. Then, the manganese dioxide is filtered off, and the filtrate is concentrated by evaporation. The crude product is chromatographed on silica gel with hexane/ethyl acetate (20 to 50%). 3.3 mg of the desired product is obtained as a red film. MS (ESI): 438 (M+H); ¹H-NMR (CDCl₃): δ=1.48 (s, 3H), 1.53 (s, 3H), 2.59 (dd, 2H), 3.13 (s, 1H), 3.24 (dd, 1H), 3.37 (dd, 1H), 3.89 (s, 3H), 4.80-4.84 (m, 1H), 6.47 (d, 1H), 6.83 (dd, 1H), 6.91-6.99 (m, 1H), 7.05 (dd, 1H), 7.76-7.87 (m, 2H), 8.11 (d, 1H), 9.08 (d, 1H).

### Example 13

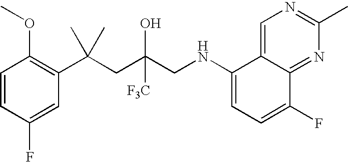

### 4-(5-Fluoro-2-methoxyphenyl)-4-methyl-1-(8-fluoro-2-methylquinazolin-5-ylamino)-2-(trifluoromethyl)-pentan-2-ol

5-Amino-8-fluoro-2-methylquinazoline

A solution of 2.4 g (18.6 mmol) of 2,5-difluoroaniline in 11 ml of water and 1.6 ml of concentrated hydrochloric acid (37%) that is 50° C. is added to a solution of 3.35 g (20.25 mmol) of chloral hydrate and 21.27 g (149.7 mmol) of sodium sulfate in 72 ml of water, which was already stirred at this temperature for 1 hour. It is stirred for another 30 minutes at room temperature, and after 4.09 g (58.9 mmol) of hydroxyl ammonium chloride in 19 ml of water is added, it is heated over 45 minutes to 125° C. and kept at this temperature for 5 minutes. After cooling and after another hour, the precipitated light brown precipitate is filtered off, washed with water and dried. 3.0 g (15.0 mmol) of the hydroxylimine is obtained as an intermediate product, which is dissolved in portions in 15 ml of concentrated sulfuric acid at 60° C. After the addition is completed, it is heated for 2 hours to 80° C. and for 4 hours to 90° C. It is allowed to cool off, and the solution is poured onto 100 g of ice. It is extracted with ethyl acetate, the organic phase is washed with water, dried on sodium sulfate and concentrated by evaporation. After chromatography on silica gel with hexane-ethyl acetate (0-45%), 1.2 g (7.1 mmol) of the 4,7-difluoroisatin is obtained. 1.8 ml of a 30% hydrogen peroxide solution is added in drops to the isatin in 30 ml of a 1 molar sodium hydroxide solution over 10 minutes. After 2 hours of stirring at room temperature, it is cooled to 0° C., and 5 ml of a 4 molar hydrochloric acid is added and diluted with 50 ml of water. It is extracted with ethyl acetate, dried on sodium sulfate, concentrated by evaporation, and 1.27 g of 3,6-difluoroanthranilic acid, which is reacted without further purification, is thus obtained quantitatively. The 3,6-difluoroanthranilic acid is heated in 8 ml of acetic acid anhydride for 45 minutes to 100° C. After cooling, the acetic acid and excess acetic acid anhydride that are produced are removed azeotropically with toluene in a vacuum. The residue is mixed with 40 ml of a 25% ammonia solution while being cooled with ice and stirred for 72 hours. It is diluted with water and acidified with acetic acid. It is extracted with ethyl acetate, the organic phase is washed with water, dried on sodium sulfate and concentrated by evaporation. The thus obtained 1.03 g (5.25 mmol) of 5,8-difluoro-2-methyl-3H-quinazolin-4-one and 6 g of phosphorus pentachloride are heated in 20 ml of phosphoryl chloride for 12 hours to 125° C. After cooling, it is poured into saturated NaHCO₃ solution and extracted with ethyl acetate. The organic phase is dried, and the solvent is removed. 1.7 g of 4-chloro-5,8-difluoro-2-methylquinazoline, which is dissolved in 60 ml of ethyl acetate and 5 ml of triethylamine, is obtained quantitatively. 600 mg of palladium is added to carbon and shaken for 2 hours (480 ml of hydrogen absorption) under a hydrogen atmosphere at normal pressure. Catalyst is removed from the solution by means of filtration on Celite, whereby it was rewashed with 100 ml of ethanol and concentrated by evaporation. After chromatography on silica gel with hexane-ethyl acetate-ethanol (0-40%), 550 mg of 5,8-difluoro-2-methylquinazoline is obtained. 890 mg (13.7 mmol) of sodium azide is added to 240 mg (1.3 mmol) of 5,8-difluoro-2-methylquinazoline and 300 mg (1.13 mmol) of 18-crown-6 in 10 ml of DMF, and the mixture is heated for 8 hours to 125° C. The solvent is removed in a vacuum, and it is chromatographed on silica gel with ethyl acetate, and 52 mg of product is obtained. ¹H-NMR (CDCl₃); δ=2.92 (s, 3H), 4.31 (br., 2H), 6.67 (dd, 1H), 7.38 (dd, 1H), 9.37 (s, 1H).

In dichloroethane, 50 mg of sodium acetate, 0.05 ml of trifluoroacetic acid and 0.1 ml of acetic acid are added to 200 mg (0.48 mmol) of 4-(5-fluoro-2-methoxyphenyl)-2-hydroxy-4-methyl-2-(trifluoromethyl)pentanal and 40 mg (0.23 mmol) of 5-amino-8-fluoro-2-methylquinazoline. It is refluxed, and after 4 hours, the solvent is removed in a vacuum with the addition of toluene. After chromatography on silica gel with hexane-ethyl acetate (0-70%), 58 mg of 4-(5-fluoro-2-methoxyphenyl)-4-methyl-1-(8-fluoro-2-methylquinazolin-5-ylimino)-2-(trifluoromethyl)-pentan-2-ol is obtained. 20 mg of palladium on carbon is added to the imine in 10 ml of ethyl acetate and 1 ml of triethylamine, and it is shaken for 1 hour under hydrogen atmosphere at normal pressure. Catalyst is removed from the solution by means of filtration, and it is concentrated by evaporation. It is taken up in 5 ml of chloroform, and 200 mg of activated manganese dioxide is added, and it is stirred for 30 minutes. It is filtered on Celite and concentrated by evaporation in a vacuum. After chromatography on silica gel with hexane-ethyl acetate (0-70%), 12 mg of the product is obtained. ¹H-NMR (CDCl₃); δ=1.46 (s, 3H), 1.55 (s, 3H), 2.37 (d, 1H), 2.76 (d, 1H), 2.90 (s, 3H), 3.13 (dd, 1H), 3.27 (dd, 1H), 3.85 (s, 3H), 4.50 (br., 1H), 5.94 (dd, 1H), 6.77 (dd, 1H), 6.91 (ddd, 1H), 7.08 (dd, 1H), 7.30 (dd, 1H), 9.16 (s, 1H).

### Example 14

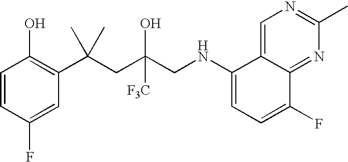

### 4-(5-Fluoro-2-hydroxyoxyphenyl)-4-methyl-1-(8-fluoro-2-methylquinazolin-5-ylamino)-2-(trifluoromethyl)-pentan-2-ol

Analogously to Example 3, 20 mg (43 μmol) of 4-(5-fluoro-2-methoxyphenyl)-4-methyl-1-(8-fluoro-2-methylquinazolin-5-ylamino)-2-(trifluoromethyl)-pentan-2-ol in 4 ml of CH₂Cl₂ is reacted with 2 ml of 1 M boron tribromide-CH₂Cl₂ solution. After chromatography on silica gel with hexane/2-propanol (10%), 17 mg of the product is obtained. ¹H-NMR (CDCl₃); δ=1.50 (s, 3H), 1.57 (s, 3H), 2.35 (d, 1H), 2.86 (s, 3H), 2.90 (d, 1H), 3.21 (dd, 1H), 3.36 (dd, 1H), 4.72 (br., 1H), 6.08 (dd, 1H), 6.54 (dd, 1H), 6.68 (ddd, 1H), 7.03 (dd, 1H), 7.33 (dd, 1H), 9.19 (s, 1H).

### Example 15

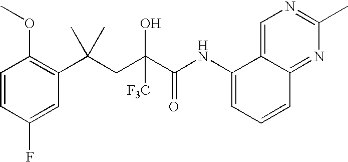

### N-(2-Methylquinazolin-5-yl)-4-(5-fluoro-2-methoxyphenyl)-2-hydroxy-4-methyl-2-(trifluoromethyl)pentanoic acid amide

104 mg (0.41 mmol) of 4-(5-fluoro-2-methoxyphenyl)-4-methyl-2-oxopentanoic acid (WO 00/32584) and 100 mg (0.63 mmol) of 5-amino-2-methylquinazoline in 2 ml of DMF are mixed at room temperature under argon with 102 mg (4.49 mmol) of dicyclohexylcarbodiimide. It is allowed to stir for 3 hours at room temperature, the reaction mixture is poured into water, extracted with ethyl acetate, the organic phase is washed with water and dried (Na₂SO₄). After chromatography on silica gel with hexane-ethyl acetate (0-70%), 64.9 mg of N-(2-methylquinazolin-5-yl)-4-(5-fluoro-2-methoxyphenyl)-4-methyl-2-oxopentanoic acid amide is obtained, which is dissolved in 2.2 ml of DMF and cooled to 0° C. The solution is mixed with 0.18 ml of (trifluoromethyl)trimethylsilane and 243 mg of cesium carbonate and stirred for 6 hours at room temperature. Water is added, it is extracted with ethyl acetate, the organic phase is washed with water and dried on sodium sulfate. The intermediate product that is concentrated by evaporation is taken up in 2 ml of THF, and 100 μl of a 1 M solution of tetrabutylammonium fluoride is added. It is stirred for 30 minutes, water is added, it is extracted with ethyl acetate, the organic phase is washed with water and dried on sodium sulfate. After chromatography on silica gel with hexane-ethyl acetate (0-65%), 14.7 mg of product is obtained. ¹H-NMR (CDCl₃); δ=1.44 (s, 3H), 1.46 (s, 3H), 2.85 (d, 1H), 2.91 (s, 1H), 3.04 (d, 1H), 3.89 (s, 3H), 4.18 (s, 1H), 6.77 (m, 2H), 6.94 (dd, 1H), 7.79 (d, 1H), 7.86 (t, 1H), 8.05 (d, 1H), 9.08 (s, 1H), 9.12 (s, 1H).

### Example 16

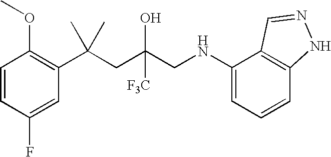

### 4-(5-Fluoro-2-methoxyphenyl)-1-(1H-indazol-4-ylamino)-4-methyl-2-(trifluoromethyl)pentan-2-ol

154 mg of 4-(5-fluoro-2-methoxyphenyl)-2-hydroxy-4-methyl-2-(trifluoromethyl)pentanal and 80 mg of 1H-indazol-4-ylamine (from Auwers Chem. Ber., 1920, 53, 1213) are dissolved in 10 ml of toluene and 1.5 ml of acetic acid and stirred for 16 hours at room temperature. It is mixed with ethyl acetate and sodium bicarbonate solution, the ethyl acetate phase is washed twice with sodium bicarbonate solution, dried on sodium sulfate and concentrated by evaporation. After chromatography on silica gel with hexane/ethyl acetate (1.5+1), 172 mg of 4-(5-fluoro-2-methoxyphenyl)-1-(1H-indazol-4-ylimino)-4-methyl-2-(trifluoromethyl)pentan-2-ol is obtained. MS(EI⁺): 423/424.

148 mg of imine is dissolved in 5 ml of methanol and 0.5 ml of acetic acid, combined with 60 mg of sodium cyanoborohydride, and stirred for 2 hours at 0° C. and for 6 hours at room temperature. It is mixed with ethyl acetate and sodium bicarbonate solution, the ethyl acetate phase is washed twice with sodium bicarbonate solution, dried and concentrated by evaporation. After chromatography on silica gel with hexane/ethyl acetate (1.5+1), 130 mg of 4-(5-fluoro-2-methoxyphenyl)-1-(1H-indazol-4-ylamino)-4-methyl-2-(trifluoromethyl)pentan-2-ol is obtained. MS(EI⁺): 425/426, ¹H-NMR (CDCl₃); δ=1.45 (s, 3H), 1.58 (s, 3H), 2.27 (d, 1H), 2.78 (d, 1H), 3.18 (d, 1H), 3.35 (d, 1H), ), 3.85 (s, 3H), 5.67 (d, 1H), 6.83 (dd, 1H), 6.85 (d, 1H), 6.95 (ddd, 1H), 7.12 (dd, 1H), 7.15 (dd, 1H), 7.86 (br, 1H).

### Example 17

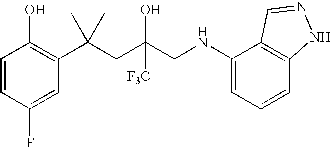

### 4-(5-Fluoro-2-hydroxyphenyl)-1-(1H-indazol-4-ylamino)-4-methyl-2-(trifluoromethyl)pentan-2-ol

Analogously to Example 3, 127 mg of 4-(5-fluoro-2-methoxyphenyl)-1-(1H-indazol-4-ylamino)-4-methyl-2-(trifluoromethyl)pentan-2-ol is reacted with 10 ml of 1 M boron tribromide-CH₂Cl₂ solution. After chromatography on silica gel with hexane/ethyl acetate (40%), 60 mg of 4-(5-fluoro-2-hydroxyphenyl)-1-(1H-indazol-4-ylamino)-4-methyl-2-(trifluoromethyl)pentan-2-ol is obtained. Flash point: 164-165° C. MS(EI⁺): 411/412 ¹H-NMR (D6-DMSO); δ=1.37 (s, 3H), 1.55 (s, 3H), 1.92 (d, 1H), 2.92 (dd, 1H), 3.03-3.18 (2H), 5.16 (t(br), 1H), 5.58 (d, 1H), 5.82 (s, 1H), 6.65 (d, 1H), 6.81 (dd, 1H), 6.85 (ddd, 1H), 6.95 (dd, 1H), 7.00 (dd, 1H), 7.97 (s, 1H), 9.75 (s, 1H), 12.7 (s, 1H)

### Example 18

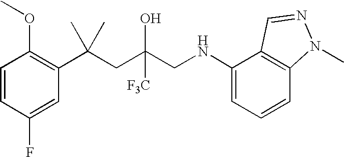

### 4-(5-Fluoro-2-methoxyphenyl)-4-methyl-1-(1-methyl-1H-indazol-4-ylamino)-2-(trifluoromethyl)pentan-2-ol

154 mg of 4-(5-fluoro-2-methoxy-phenyl)-2-hydroxy-4-methyl-2-(trifluoromethyl)pentanal and 91 mg of 1-methyl-1H-indazol-4-ylamine (Sureau Chimia, 1961, 15, 195) are reacted, as described in Example 15, to form 4-(5-fluoro-2-methoxyphenyl)-4-methyl-1-(1-methyl-1H-indazol-4-ylimino)-2-(trifluoromethyl)pentan-2-ol MS(EI⁺): 437/438 and further reduced with sodium cyanoborohydride to 4-(5-fluoro-2-methoxyphenyl)-4-methyl-1-(1-methyl-1H-indazol-4-ylamino)-2-(trifluoromethyl)pentan-2-ol. MS(EI⁺): 439/440, ¹H-NMR (CDCl₃); δ=1.46 (s, 3H), 1.59 (s, 3H), 2.27 (d, 1H), 2.77 (d, 1H), 3.05-3.20 (3H), 3.38 (d, 1H), 3.82 (s, 3H), 4.00 (s, 3H), 5.60 (d, 1H), 6.75 (d, 1H), 6.84 (dd, 1H), 6.95 (ddd, 1H), 7.12 (dd, 1H), 7.16 (dd, 1H), 7.75 (s, 1H).

### Example 19

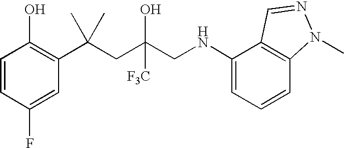

### 4-(5-Fluoro-2-hydroxyphenyl)-4-methyl-1-(1-methyl-1H-indazol-4-ylamino)-2-(trifluoromethyl)pentan-2-ol

Analogously to Example 3, 22 mg of 4-(5-fluoro-2-hydroxyphenyl)-4-methyl-1-(1-methyl-1H-indazol-4-ylamino)-2-(trifluoromethyl)pentan-2-ol is obtained from 84 mg of 4-(5-fluoro-2-methoxyphenyl)-4-methyl-1-(1-methyl-1H-indazol-4-ylamino)-2-(trifluoromethyl)pentan-2-ol. Flash point: 193-194° C., MS(EI⁺): 425/426, ¹H-NMR (D6-DMSO); δ=1.40 (s, 3H), 1.53 (s, 3H), 1.91 (d, 1H), 2.95 (dd, 1H), 3.09-3.20 (2H), 3.90 (s, 3H), 5.26 (t(br), 1H),), 5.62 (d, 1H), 5.83 (s, 1H), 6.73 (d, 1H), 6.80 (dd, 1H), 6.85 (ddd, 1H), 6.99-7.05 (2H), 7.93 (s, 1H), 9.75 (s, 1H)

### Example 20

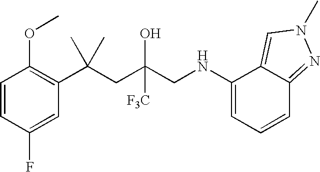

### 4-(5-Fluoro-2-methoxyphenyl)-4-methyl-1-(2-methyl-2H-indazol-4-ylamino)-2-(trifluoromethyl)pentan-2-ol

154 mg of 4-(5-fluoro-2-methoxy-phenyl)-2-hydroxy-4-methyl-2-(trifluoromethyl)pentanal and 91 mg of 2-methyl-2H-indazol-4-ylamine (Sureau Chimia, 1961, 15, 195) are reacted, as described in Example 15, to form 4-(5-fluoro-2-methoxyphenyl)-4-methyl-1-(2-methyl-2H-indazol-4-ylimino)-4-methyl-2-(trifluoromethyl)pentan-2-ol [flash point: 92-94° C., MS(EI⁺): 437/438] and further reduced with sodium cyanoborohydride to 4-(5-fluoro-2-methoxyphenyl)-4-methyl-1-(2-methyl-2H-indazol-4-ylamino)-2-(trifluoromethyl)pentan-2-ol. MS(EI⁺): 439/440, ¹H-NMR (CDCl₃); δ=1.47 (s, 3H), 1.56 (s, 3H), 2.30 (d, 1H), 2.75 (d, 1H), 3.14 (d, 1H), 3.29 (d(br), 1H), 3.33 (s(br), 1H), 3.75 (s(br), 1H), 3.85 (s, 3H), 4.15 (s, 3H), 5.55 (d, 1H), 6.86 (d, 1H), 6.95-7.07 (2H), 7.10 (dd, 1H), 7.15 (dd, 1H), 7.66 (s, 1H)

### Example 21

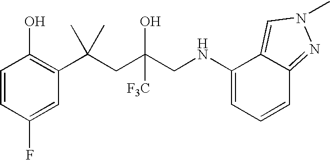

### 4-(5-Fluoro-2-hydroxyphenyl)-4-methyl-1-(2-methyl-2H-indazol-4-ylamino)-2-(trifluoromethyl)pentan-2-ol

Analogously to Example 3, 100 mg of 4-(5-fluoro-2-hydroxyphenyl)-4-methyl-1-(2-methyl-2H-indazol-4-ylamino)-2-(trifluoromethyl)pentan-2-ol is obtained from 132 mg of 4-(5-fluoro-2-methoxyphenyl)-4-methyl-1-(2-methyl-2H-indazol-4-ylamino)-2-(trifluoromethyl)pentan-2-ol. Flash point: 182° C., MS(EI⁺): 425/426, ¹H-NMR (D6-DMSO); δ=1.40 (s, 3H), 1.55 (s, 3H), 1.93 (d, 1H), 2.89 (dd, 1H), 3.05-3.17 (2H), 4.07 (s, 3H), 5.00 (t(br), 1H), 5.38 (d, 1H), 5.85 (s, 1H), 6.72-6.90 (4H), 6.99 (dd, 1H), 8.16 (s, 1H), 9.73 (s, 1H)

### Example 22

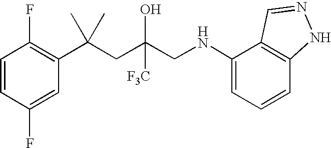

### 4-(2,5-Difluorophenyl)-1-(1H-indazol-4-ylamino)-4-methyl-2-(trifluoromethyl)pentan-2-ol

Analogously to Example 3, 207 mg (0.73 mmol) of 4-(2,5-difluorophenyl)-2-hydroxy-4-methyl-2-trifluoromethyl-pentanal is reacted with 150 mg (1.10 mmol) of 1H-indazol-4-ylamine (from Auwers Chem. Ber., 1920, 53, 1213). 110 mg of 4-(2,5-difluorophenyl)-1-(1H-indazol-4-ylimino)-4-methyl-2-(trifluoromethyl)pentan-2-ol is obtained. 50 mg (0.12 mmol) thereof is reduced analogously to Example 7 with 27 mg (0.72 mmol) of NaBH₄. After chromatography on silica gel with hexane/ethyl acetate (20-30%), 18 mg of the desired product is obtained.

¹H-NMR (300 MHz, CDCl₃): □=1.48 (s, 3H), 1.62 (s, 3H), 2.27 (d, 1H), 2.52 (d, 1H), 3.23-3.38 (m, 2H), 5.67 (d, 1H), 6.83-7.01 (m, 3H), 7.08-7.18 (m, 2H), 7.89 (s, 1H)

### Example 23

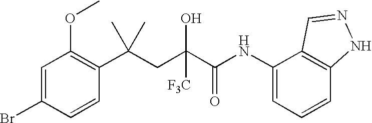

### 4-(4-Bromo-2-methoxyphenyl)-2-hydroxy-N-(1H-indazol-4-yl)-4-methyl-2-(trifluoromethyl)pentanoic acid amide

122 mg of 4-dimethylaminopyridine is dissolved in the heat in 3 ml of Sulfolan®, cooled to room temperature and combined with 0.0525 ml of thionyl chloride. After 45 minutes at room temperature, it is mixed with 192 mg of 4-(4-bromo-2-methoxyphenyl)-2-hydroxy-4-methyl-2-(trifluoromethyl)pentanoic acid (WO 98/54159) and stirred again for 45 minutes at room temperature. It is mixed with 90 mg of 1H-indazol-4-ylamine (from Auwers Chem. Ber., 1920, 53, 1213), heated for 1 hour to 80° C. and combined with sodium bicarbonate solution and ethyl acetate. The ethyl acetate phase is washed four times with water, dried and concentrated by evaporation. After chromatography on silica gel with hexane/ethyl acetate (50%), 150 mg of 4-(4-bromo-2-methoxyphenyl)-2-hydroxy-N-(1H-indazol-4-yl)-4-methyl-2-(trifluoromethyl)pentanoic acid amide is obtained. MS(EI⁺): 499/501 ¹H-NMR (D6-DMSO); δ=1.38 (s, 3H), 1.50 (s, 3H), 2.17 (d, 1H), 3.10 (d, 1H), 3.83 (s, 3H), 6.65 (dd, 1H), 6.97 (d, 1H), 7.03 (d, 1H), 7.08 (s, 1H), 7.10 (d, 1H), 7.27 (dd, 1H), 7.31 (d, 1H), 7.92 (s, 1H), 9.45 (s, 1H), 13.1 (s, 1H)

### Example 24

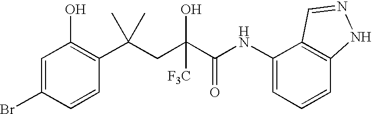

### 4-(4-Bromo-2-hydroxyphenyl)-2-hydroxy-N-(1H-indazol-4-yl)-4-methyl-2-(trifluoromethyl)pentanoic acid amide

Analogously to Example 3, 55 mg of 4-(4-bromo-2-hydroxyphenyl)-2-hydroxy -N-(1H-indazol-4-yl)-4-methyl -2-(trifluoromethyl)pentanoic acid amide is obtained from 100 mg of 4-(4-bromo-2-methoxyphenyl)-2-hydroxy-N-(1H-indazol-4-yl)-4-methyl-2-(trifluoromethyl)pentanoic acid amide. MS(EI⁺): 485/487, ¹H-NMR (D6-DMSO); δ=1.42 (s, 3H), 1.48 (s, 3H), 2.23 (d, 1H), 3.15 (d, 1H), 6.54 (dd, 1H), 6.83 (d, 1H), 6.95 (d, 1H), 7.00 (s, 1H), 7.10 (d, 1H), 7.25 (dd, 1H), 7.30 (d, 1H), 7.95 (s, 1H), 9.61 (s, 1H), 9.95 (s, 1H), 13.12 (s, 1H)

### Example 25

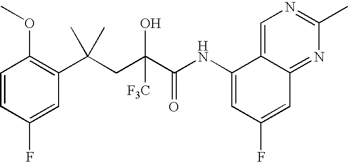

### 4-(5-Fluoro-2-methoxyoxyphenyl)-4-methyl-1-(7-fluoro-2-methylquinazolin-5-ylamino)-2-(trifluoromethyl)-pentan-2-ol

5-Amino-7-fluoro-2-methylquinazoline

17 g (70.5 mmol) of 3,6-difluoro-2-N-pivaloylaminobenzaldehyde (L. Florvall, I. Fagervall, L.-G- Larsson, S. B. Ross, Eur. J. Med. Chem. 34 (1999) 137-151), 9.2 g of acetamidine hydrochloride, 13.4 g of potassium carbonate and 10.4 g of molecular sieve (4A) are added together in 70 ml of butyronitrile. It is heated for 17 hours to 145° C. while being stirred vigorously, and the solvent is removed in a vacuum. After chromatography of the residue on silica gel with hexane/ethyl acetate (0-70%), 4.5 g of 7-fluoro-5-N-pivaloylamino-2-methylquinazoline is obtained.

1 g (3.82 mmol) of 7-fluoro-5-N-pivaloylamino-2-methylquinazoline is dissolved in 74 ml of toluene and cooled to −70° C. Over 30 minutes, 9.5 ml (11.4 mmol) of a 1.2 M diisobutyl aluminum hydride solution in toluene is added in drops. The reaction mixture is allowed to heat to −40° C. and stirred for 4 hours at −40° C. Water is slowly added, and it is stirred for 30 minutes at room temperature until a precipitate forms, which is removed by means of filtration through Celite. The phases are separated, washed with saturated sodium chloride solution and dried on sodium sulfate. After chromatography on silica gel with hexane-ethyl acetate (0-100%), 64 mg of the product is obtained. ¹H-NMR (CDCl₃); δ=2.83 (s, 3H), 4.67 (br., 2H), 6.50 (dd, 1H), 6.93 (dd, 1H), 9.23 (s, 1H).

0.1 ml of titanium tetraethylate is added to 22 mg (0.07 mmol) of 4-(5-fluoro-2-methoxyphenyl)-2-hydroxy-4-methyl-2-(trifluoromethyl)-pentanal and 11 mg (0.06 mmol) of 5-amino-7-fluoro-2-methylquinazoline in 4 ml of toluene, and the mixture is heated over 2.5 hours to 100° C. After cooling, it is poured into water, and vigorous stirring is continued. The suspension is filtered through Celite, and rewashed thoroughly with ethyl acetate. The phases of the filtrate are separated, and it is extracted again with ethyl acetate. It is dried on sodium sulfate, and the solvent is removed in a vacuum. The 4-(5-fluoro-2-methoxyphenyl)-1-(7-fluoro-2-methylquinazolin-5-ylimino)-4-methyl-2-(trifluoromethyl)-pentan-2-ol that is thus obtained in crude form is in 8 ml of ethyl acetate, in 5 ml of ethyl acetate and 0.5 ml of triethylamine, 20 mg of palladium on carbon is added, and it is shaken for 1 hour under a hydrogen atmosphere at normal pressure. Catalyst is removed from the solution by means of filtration, and it is concentrated by evaporation. It is taken up in 5 ml of chloroform, 50 mg of activated manganese dioxide is added, and it is stirred for 20 minutes. It is filtered on Celite and concentrated by evaporation in a vacuum. After chromatography on silica gel with hexane-ethyl acetate (0-70%), 18 mg of the product is obtained. ¹H-NMR (CDCl₃); δ=1.47 (s, 3H), 1.54 (s, 3H), 2.44 (d, 1H), 2.70 (d, 1H), 2.81 (s, 3H), 3.15 (dd, 1H), 3.30 (dd, 1H), 3.86 (s, 3H), 4.97 (br., 1H), 5.83 (dd, 1H), 6.79 (dd, 1H), 6.85 (dd, 1H), 6.92 (ddd, 1H), 7.06 (dd, 1H), 8.98 (s, 1H).

### Example 26

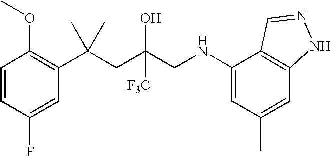

### 4-(5-Fluoro-2-methoxyphenyl)-1-(6-methoxyl-1H-indazol-4-ylamino)-4-methyl-2-(trifluoromethyl)pentan-2-ol

Analogously to Example 16, the desired product is obtained from 4-(5-fluoro-2-methoxyphenyl)-2-hydroxy-4-methyl-2-(trifluoromethyl)pentanal and 6-methyl-1H-indazol-4-ylamine (from Auwers Chem. Ber., 1920, 53, 1213). ¹H-NMR (CD₃OD); δ=1.45 (s, 3H), 1.64 (s, 3H), 2.01 (d, 1H), 2.23 (s, 3H), 2.78 (d, 1H), 3.01 (d, 1H), 3.21 (d, 1H), 3.86 (s, 3H), 5.96 (s, 1H), 6.93 (m, 2H), 7.14 (dd, 1H), 7.21 (s, 1H), 7.73 (s, 1H).

### Example 27

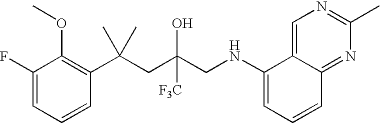

### 4-(3-Fluoro-2-methoxyphenyl)-1-(2-methylquinazolin-5-ylamino)-4-methyl-2-(trifluoromethyl)pentan-2-ol

2,6-Difluoroanisole

20 g (153.74 mmol) of 2,6-difluorophenol is dissolved in 200 ml of acetone and mixed under nitrogen with 42.5 g (307.48 mmol) of potassium carbonate. After 19.1 ml of methyl iodide (2 equivalents) is added, it is refluxed for three and one-half hours. After cooling, the reaction mixture is filtered, the filter residue is washed with acetone, and the filtrate is spun in until a dry state is reached. The residue is chromatographed on silica gel (mobile solvent ethyl acetate/hexane). 17.27 g (77.9%) of the desired product is obtained. It should be noted that the product is slightly volatile. The bath temperature should not exceed 30° C. and is to be adapted to the vacuum of the rotary evaporator.

2-(3-Fluoro-2-methoxyphenyl)-2-methylpropanenitrile

10 g (69.39 mol) of 2,6-difluoroanisole is dissolved in 200 ml of toluene and mixed at room temperature with 5.75 g (83.27 mmol) of isobutyric acid nitrile. Within 35 minutes, 166.5 ml of a 0.5 molar solution of potassium hexamethyl disilazide in toluene is added in drops. In this case, a slight temperature rise to 27.5° C. takes place. After 16 hours of stirring at room temperature, the reaction mixture is mixed with 200 ml of water and 400 ml of ethyl acetate and acidified with 10% sulfuric acid to a pH of 4. The organic phase is separated, and the aqueous phase is shaken once with ethyl acetate (200 ml). The combined organic extracts are shaken with water and brine. After the solvent is dried, filtered, and spun off, the residue is chromatographed on silica gel (mobile solvent ethyl acetate/hexane). 7.66 g (57.1%) of the desired compound is isolated.

2-(3-Fluoro-2-methoxyphenyl)-2-methylpropanal

7.66 g (39.64 mmol) of the above-described nitrile is dissolved in 158 ml of toluene. At −65 to −60° C. and within 40 minutes, 49.5 ml of a 1.2 molar solution of DIBAH in toluene is added in drops. After one hour of stirring at this temperature is begun, 493 ml of a 10% L-(+)-tartaric acid solution is to be added in drops. After 100 milliliters, the temperature is increased to −10° C. The remainder of the tartaric acid solution is quickly added, and the batch is stirred vigorously for two hours at room temperature. The reaction mixture is shaken twice with 400 ml of diethyl ether each. The combined organic extracts are shaken with water and brine, dried, and the solvent is spun off. The residue that is obtained (7.8 g=102%) is incorporated in crude form into the next stage.

(E/Z)-4-(3-Fluoro-2-methoxyphenyl)-4-methylpent-2-enoic acid ethyl ester

21.3 ml of a 2 molar LDA solution in THF is added in drops to a solution of 9.87 g (39.75 mmol) of 2-ethoxy-phosphonoacetic acid triethyl ester in 40 ml of absolute THF at 0° C. After 30 minutes of stirring at 0° C., 7.8 g (39.75 mmol) of 2-(3-fluoro-2-methoxyphenyl)-2-methylpropanal, dissolved in 26 ml of THF, is quickly added in drops at 0° C. The cold bath is removed, and the batch is stirred for 16 hours at room temperature. The reaction mixture is poured into water and extracted twice with ethyl acetate. The combined organic extracts are washed with water and brine, dried, and after the desiccant is filtered off, the solvent is spun off. The residue is chromatographed on silica gel (mobile solvent ethyl acetate/hexane). 8.39 g (68.2%) of the desired compound is isolated.

(E/Z)-4-(3-Fluoro-2-methoxyphenyl)-4-methylpent-2-enoic acid

8.39 g (27.03 mmol) of (E/Z)-4-(3-fluoro-2-methoxyphenyl)-4-methylpent-2-enoic acid ethyl ester is mixed with 270 ml of 1 N NaOH in ethanol/water (2:1) and stirred for two days at room temperature. The ethanol is drawn off in a rotary evaporator, and the residue is extracted twice with 150 ml of diethyl ether each. The combined organic extracts are washed with water and discarded after monitoring by TLC. The aqueous phases are acidified with concentrated hydrochloric acid to pH 3 and extracted twice with 300 ml each of diethyl ether. The ether extracts are washed with water and brine, dried, the solvent is spun off, and the residue (5.89 g=77.2%) is incorporated in crude form into the next stage.

4-(3-Fluoro-2-methoxyphenyl)-4-methyl-2-oxo-pentanoic acid

5.89 g (20.86 mmol) of (E/Z)-4-(3-fluoro-2-methoxyphenyl)-4-methylpent-2-enoic acid is mixed at room temperature with 126 ml of a I molar sulfuric acid, and after 21 ml of glacial acetic acid is added, it is stirred for 15 hours at a bath temperature of 90° C. The reaction mixture is mixed with solid potassium carbonate to a pH of 9 while being cooled carefully in an ice bath (heavily foaming). It is extracted twice with diethyl ether. The combined organic extracts are washed with water and discarded after TLC. The combined aqueous phases are acidified with concentrated hydrochloric acid to a pH of 4 and extracted twice with 300 ml each of diethyl ether. The ether extracts are washed with water and brine, dried, and the solvent is spun off. Since the residue still contains acetic acid, it is spun off twice with 100 ml each of toluene. The remaining residue (4.14 g=78.1%) is incorporated in crude form into the next stage.

4-(3-Fluoro-2-methoxyphenyl)-4-methyl-2-oxo-pentanoic acid ethyl ester

4.14 g (16.28 mmol) of 4-(3-fluoro-2-methoxyphenyl)-4-methyl-2-oxo-pentanoic acid is dissolved in 97 ml of ethanol, mixed with 1.79 ml of sulfuric acid, and refluxed for four hours. The ethanol is drawn off in a rotary evaporator, and the residue is carefully mixed with co-saturated sodium bicarbonate solution until a pH of 9 is reached. It is extracted twice with 100 ml each of ethyl acetate, and the combined organic extracts are washed with water and then with brine. After the desiccant is dried and filtered off, and after the solvent is spun in, the residue is chromatographed on silica gel (mobile solvent ethyl acetate/hexane). 4.16 g (90.6%) of the desired compound is isolated.

4-(3-Fluoro-2-methoxyphenyl)-4-methyl-2-trifluoromethyl-2-trimethylsilyloxy-pentanoic acid ethyl ester

4.16 g (14.74 mmol) of 4-(3-fluoro-2-methoxyphenyl)-4-methyl-2-oxo-pentanoic acid ethyl ester is dissolved in 24 ml of THF and mixed at 0° C. with 2.51 g (17.68 mmol) of (trifluoromethyl)-trimethylsilane and 36.1 mg of tetrabutylammonium fluoride. After two and one-half hours of stirring between 0 and 5° C., the batch is poured into 50 ml of ice water. It is extracted twice with 150 ml each of diethyl ether, and the combined organic extracts are worked up as usual. After chromatography on silica gel (mobile solvent ethyl acetate/hexane), 5.24 g (83.8%) of the desired compound is obtained.

4-(3-Fluoro-2-methoxyphenyl)-4-methyl-2-trifluoromethyl-2-trimethylsilyloxy-pentan-1-ol

5.24 g (12.34 mmol) of 4-(3-fluoro-2-methoxyphenyl)-4-methyl-2-trifluoromethyl-2-trimethylsilyloxy-pentanoic acid ethyl ester is dissolved in 45 ml of diethyl ether and mixed at 0 to 5° C. in portions with 936.9 mg (24.69 mmol) of LiAlH₄. After four and one-half hours of stirring at room temperature, the reaction mixture is carefully mixed with saturated NaHCO₃ while being cooled in an ice bath, and it is stirred for one hour under cold conditions and overnight at room temperature. After the usual working-up, 4.11 g (87.1%) of a mixture that consists of the desired compound and the compound are obtained, in which the silyl ether has migrated. The mixture is incorporated in crude form into the next stage.

4-(3-Fluoro-2-methoxyphenyl)-4-methyl-2-trifluoromethyl-pentane-1,2-diol

4.11 g (10.75 mmol) of 4-(3-fluoro-2-methoxyphenyl)-4-methyl-2-trifluoromethyl-2-trimethylsilyloxy-pentan-1-ol is dissolved in 61 ml of THF, mixed with 3.39 g (10.746 mmol) of Bu₄NF trihydrate, and stirred for one hour at room temperature. The reaction mixture is poured into water and extracted twice with diethyl ether. The organic phases are washed as usual with water and brine. After the desiccant is dried and filtered off, and after the solvent is spun in, the remaining residue is chromatographed on silica gel (mobile solvent ethyl acetate/hexane). 2.71 g (81.4%) of the desired compound is isolated.

4-(3-Fluoro-2-methoxyphenyl)-2-hydroxy-4-methyl-2-trifluoromethyl-pentanal

765 mg (6.03 mmol) of oxalyl chloride in 13 ml of dichloromethane is introduced into a heated flask. At −78° C., 0.855 ml of DMSO, dissolved in 2.5 ml of dichloromethane, is added in drops, and the batch is stirred for five more minutes. Then, 1.7 g (5.48 mmol) of 4-(3-fluoro-2-methoxyphenyl)-4-methyl-2-trifluoromethyl-pentane-1,2-diol, dissolved in 5 ml of dichloromethane, is added in drops. After 15 minutes of stirring, the batch is carefully mixed with 3.79 ml (27.40 mmol) of triethylamine, stirred for five minutes at −78° C. and slowly allowed to come to room temperature. 20 ml of water is added, and the batch is stirred for another hour at room temperature. After phase separation, the aqueous phase is shaken once with 100 ml of dichloromethane. The combined organic extracts are washed with 1% sulfuric acid, 5% sodium bicarbonate solution and brine. According to the usual procedure, 1.617 g (96.2%) of the aldehyde is obtained, which is incorporated in crude form into the next stage.

Analogously to Example 25, the desired product is obtained from 4-(3-fluoro-2-methoxyphenyl)-2-hydroxy-4-methyl-2-(trifluoromethyl)pentanal and 5-amino-2-methylquinazoline. ¹H-NMR (300 MHz, CDCl₃): □=1.45 (3H), 1.57 (3H), 2.35 (1H), 2.75 (1H), 2.82 (3H), 3.00-3.40 (3H), 4.00 (3H), 4.75 (1H), 6.10 (1H), 6.90-7.02 (2H), 7.05-7.18 (1H), 7.25 (1H), 7.55 (1H), 9.10 (1H).

### Example 28

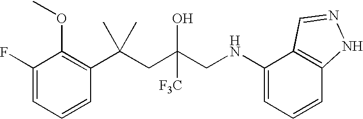

### 4-(3-Fluoro-2-methoxyphenyl)-1-(1H-indazol-4-ylamino)-4-methyl-2-(trifluoromethyl)pentan-2-ol

Analogously to Example 16, the desired product is obtained from 4-(3-fluoro-2-methoxyphenyl)-2-hydroxy-4-methyl-2-(trifluoromethyl)pentanal and 1H-indazol-4-ylamine. ¹H-NMR (300 MHz, CDCl₃): □=1.49 (3H), 1.60 (3H), 2.30 (1H), 2.71 (1H), 3.18 (1H), 3.30 (1H), 4.05 (3H), 5,75 (1H), 6.85 (1H), 6.90-7.20 (4H), 7.80 (1H).

### Example 29

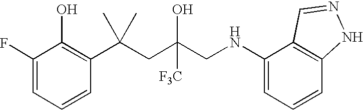

### 4-(3-Fluoro-2-hydroxyphenyl)-1-(1H-indazol-4-ylamino)-4-methyl-2-(trifluoromethyl)pentan-2-ol

Analogously to Example 3, the desired product is obtained from 4-(3-fluoro-2-methoxyphenyl)-1-(1H-indazol-4-ylamino)-4-methyl-2-(trifluoromethyl)pentan-2-ol.

¹H-NMR (300 MHz, DMSO-d₆): □=1.42 (3H), 1.59 (3H), 1.95 (1H), 2.85-2.95 (1H), 3.03-3.18 (2H), 5.15 (1H), 5.55 (1H), 5.80 (1H), 6.65 (1H), 6.70-6.80 (1H), 6.90 (1H), 6.90-7.13 (2H), 7.95 (1H), 9.80 (1H), 12.70 (1H).

### Example 30

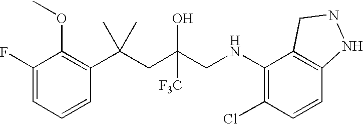

### 2-[(5-Chloro-1H-indazol-4-ylamino)-methyl]-1,1,1-trifluoro-4-(3-fluoro-2-methoxyphenyl)-4-methylpentan-2-ol

5-Chloro-4-nitro-1H-indazole

2.24 g (12 mmol) of 4-chloro-2-methyl-3-nitrophenylamine, produced according to literature (Mori et al., Chem. Pharm. Bull. 1986, 34, 4859 ff. as well as Brand and Zöller, Chem. Ber. 1907, 3324 ff.) is dissolved in 100 ml of acetic acid. At 10° C., 6.0 ml of a 2 molar aqueous sodium nitrite solution is added in drops. Then, the suspension is added within 15 minutes to boiling acetic acid (150 ml), and the reaction mixture is allowed to reflux for 4 hours. After the acetic acid is removed in a vacuum, the residue is taken up in ethyl acetate and saturated sodium bicarbonate solution. The organic phase is washed with saturated sodium chloride solution and dried on sodium sulfate. After the solvent is removed in a vacuum, the crude product (1.81 g, 76%) is further reacted.

¹H-NMR (300 MHz, DMSO-d₆): δ=7.65 (d, 1H), 7.97 (d, 1H), 8.32 (s, 1H), 13.97 (s, 1H).

5-Chloro-1H-indazol-4-ylamine

A solution that consists of 5-chloro-4-nitro-1H-indazole (872 mg, 4.41 mmol) is mixed with 150 mg of palladium on carbon (10%) and stirred under hydrogen atmosphere at room temperature. After 45 minutes, the catalyst is suctioned off on one frit and washed with methanol. The filtrate is concentrated by evaporation, and the residue is taken up in 200 ml of ethyl acetate and heated. After renewed suctioning and concentration by evaporation of the filtrate, the purification is carried out on silica gel with hexane/ethyl acetate (100-33% hexane). 296 mg (40% of theory) of the product is obtained.

¹H-NMR (300 MHz, DMSO-d₆): δ=5.97 (s, 2H), 6.66 (d, 1H), 7.05 (d, 1H), 8.19 (s, 1H), 12.83 (s, 1H).

2-[(5-Chloro-1H-indazol-4-ylimino)-methyl]-1,1,1-trifluoro-4-(3-fluoro-2-methoxyphenyl)-4-methyl-pentan-2-ol

A solution that consists of 4-(3-fluoro-2-methoxyphenyl)-2-hydroxy-4-methyl-2-trifluoro-methylpentanal (278 mg, 0.9 mmol) and 5-chloro-1H-indazol-4-ylamine (121 mg, 0.72 mmol) in 20 ml of xylene is mixed with titanium(IV)ethylate (0.42 ml, 2.0 mmol) and refluxed for 10 hours. After cooling to room temperature, xylene is distilled off, and the residue is purified on silica gel with hexane/ethyl acetate (30-100% ethyl acetate). 123 mg (37% of theory) of the product is obtained.

¹H-NMR (400 MHz, CDCl₃): δ=1.43 (s, 3H), 1.57 (s, 3H), 2.38 (d, 1H), 3.22 (d, 1H), 3.94 (d, 3H), 4.91 (s, 1H), 6.41-6.52 (m, 2H), 6.90 (d, 1H), 7.28 (d, 1H), 7.38 (d, 1H), 7.56 (s, 1H), 7.72 (s, 1H), 10.26 (br, 1H).

2-[(5-Chloro-1H-indazol-4-ylamino)-methyl]-1,1,1-trifluoro-4-(3-fluoro-2-methoxyphenyl)-4-methylpentan-2-ol

A solution that consists of 2-[(5-chloro-1H-indazol-4-ylimino)-methyl]-1,1,1-trifluoro-4-(3-fluoro-2-methoxyphenyl)-4-methylpentan-2-ol (49 mg, 0.11 mmol) in 5.0 ml of methanol is mixed with 15 mg of sodium borohydride and stirred at room temperature. Then, a total of 300 mg of sodium borohydride was added in portions within 4 days. The reaction mixture is neutralized with 10% acetic acid. After the solvent is removed, the residue is taken up in saturated sodium bicarbonate solution and ethyl acetate. It is extracted with ethyl acetate, the combined organic phases are washed with saturated sodium chloride solution and dried on sodium sulfate. After the solvent is removed in a vacuum and after the residue is purified by means of preparative thin-layer chromatography on silica gel with hexane/ethyl acetate (50% ethyl acetate), 24 mg (47%) of the product is obtained.

¹H-NMR (300 MHz, CDCl₃): δ=1.48 (s, 3H), 1.59 (s, 3H), 2.39 (d, 1H), 2.72 (d, 1H), 3.55-3.59 (m, 2H), 4.01 (d, 3H), 4.80-4.84 (m, 1H), 6.80 (d, 1H), 6.86-6.98 (m, 2H), 7.11 (d, 1H), 7.22 (d, 1H), 7.70 (s, 1H).

### Example 31

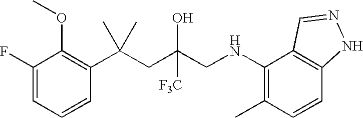

### 1,1,1-Trifluoro-4-(3-fluoro-2-methoxyphenyl)-4-methyl-2-[(5-methyl-1H-indazol-4-ylamino)-methyl]-pentan-2-ol

5-Methyl-1H-indazol-4-ylamine

It is mixed with 5.0 ml of fuming nitric acid at 0° C. into a solution that consists of 2,4-dimethylaniline (12.4 ml, 100 mmol) in 80 ml of concentrated sulfuric acid, and it is stirred for 20 minutes at 4° C., and then for 30 minutes at room temperature. The reaction mixture is poured into 600 ml of ice water, and set at pH 10 with 5N sodium hydroxide solution. The precipitate is suctioned off, washed with water and dried. 15.72 g (95% of theory) of 2,4-dimethylnitrophenylamine is obtained as a mixture of regioisomers.

Analogously to the production of 5-chloro-4-nitro-1H-indazole, 1.14 g (57% of theory) of the product was obtained as a mixture of the two regioisomers in the reaction of 2,4-dimethylnitrophenylamine (2.0 g, 12 mmol) with 6.0 ml of a 2 molar aqueous sodium nitrite solution in acetic acid (250 ml).

MS (ES+, acetonitrile/water 1:1+0.01% formic acid): m/z(%) 178 (M+1, 100).

Analogously to the production of 5-chloro-1H-indazol-4-ylamine, the regioisomeric mixture of the previous reaction (1.0 g, 5.64 mmol) is reacted with 100 mg of palladium on carbon in methanol under hydrogen atmosphere for 16 hours at room temperature. After purification on silica gel with hexane/ethyl acetate (33% hexane, then 100% ethyl acetate), 53 mg (6% of theory) of 5-methyl-1H-indazol-4-ylamine is obtained.

¹H-NMR (300 MHz, DMSO-d₆): δ=2.12 (s, 3H), 5.41 (s, 2H), 6.57 (d, 1H), 6.90 (d, 1H), 8.10 (s, 1H), 12.5 (s, 1H).

4-(3-Fluoro-2-methoxyphenyl)-2-hydroxy-4-methyl-2-trifluoro-methylpentanal (308 mg, 1.0 mmol) and 5-methyl-1H-indazol-4-ylamine (148 mg, 1.0 mmol) are introduced into 15.0 ml of xylene and mixed with titanium(IV) ethylate (0.42 ml, 2.0 mmol). After 3 hours under reflux, the reaction mixture is allowed to cool to room temperature. After ethyl acetate and saturated sodium chloride solution are added, it is vigorously stirred for 30 minutes at room temperature. The deposited precipitate is suctioned off, the aqueous phase is separated, and the organic phase is dried on sodium sulfate. The purification is carried out by means of chromatography on silica gel with hexane/ethyl acetate (30-40% ethyl acetate). 345 mg (79% of theory) of 1,1,1-trifluoro-4-(3-fluoro-2-methoxyphenyl)-4-methyl-2-[(5-methyl-1H-indazol-4-ylimino)methyl]-pentan-2-ol is obtained.

A solution that consists of 1,1,1-trifluoro-4-(3-fluoro-2-methoxyphenyl)-4-methyl-2-[(5-methyl-1H-indazol-4-ylimino)-methyl]-pentan-2-ol (151 mg, 0.34 mmol) in 20 ml of methanol is dissolved, mixed with 30 mg of palladium on carbon (10%) and stirred at room temperature under hydrogen atmosphere. After 20 hours, 30 mg of palladium on carbon is added, and the reaction mixture is allowed to stir for another 28 hours at room temperature. The catalyst is filtered off on Celite and washed with methanol. After the filtrate is concentrated by evaporation and after the residue is purified by means of preparative thin-layer chromatography on silica gel with hexane/ethyl acetate (50% ethyl acetate), 28 mg (19% of theory) of the product is obtained. ¹H-NMR (400 MHz, CDCl₃): δ=1.47 (s, 3H), 1.60 (s, 3H), 2.18 (s, 3H), 2.37 (d, 1H), 2.70 (d, 1H), 3.48 (s, 2H), 4.00 (d, 3H), 6.88-6.97 (m, 3H), 7.07 (d, 1H), 7.11 (d, 1H), 7.67 (s, 1H).

### Example 32

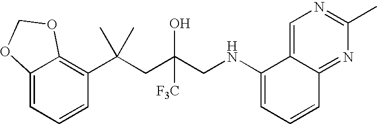

### 4-(Benzo[1,3]dioxol-4-yl)-4-methyl-1-(2-methychinazolin-5-ylamino)-2-(trifluoromethyl)-pentan-2-ol

1-(Benzo[1,3]dioxol-4-yl)-1-methylethanol

25.5 g of 4-acetylbenzo[1,3]dioxole is mixed at room temperature under argon with 57.2 ml of methylmagnesium chloride solution (3M in THF) in 375 ml THF. It is stirred for 16 hours at room temperature and added to ice/2H hydrochloric acid. It is extracted with ethyl acetate, and the organic phase is washed with water and brine, and it is dried (Na₂SO₄). 27.89 g of 1-[benzo(1,3)dioxol-4-yl]-1-methylethanol is obtained as a brown oil.

¹H-NMR (CDCl₃), □ (ppm)=1.6 (s, 6H), 5.95 (s, 2H), 6.76 (dd, 1H), 6.82 (t, 1H), 6.91 (dd, 1H)

4-(Benzo[1,3]dioxol-4-yl)-4-methyl-2-oxo-pentanoic acid ethyl ester

5.0 g of 1-(benzo[1,3]dioxol-4-yl)-1-methylethanol and 7.8 g of 2-trimethylsilyloxy-acrylic acid-ethyl ester are mixed in 100 ml of dichloromethane at −70° C. with 2.4 ml of tin(IV) chloride. After 5 minutes, the solution is added to potassium carbonate solution and vigorously stirred. It is filtered through diatomaceous earth, the phases are separated, and the aqueous phase is extracted with dichloromethane. The organic phase is washed with water and brine, dried (Na₂SO₄) and concentrated by evaporation. After chromatography on silica gel (hexane/ethyl acetate 0->10%), 3.4 g of the title compound is obtained as a colorless oil.

¹H-NMR (CDCl₃), □ (ppm)=1.25 (t, 3H), 1.44 (s, 6H), 3.31 (s, 2H), 4.12 (q, 2H), 5.92 (s, 2H), 6.7-6.82 (m, 3H)

4-(Benzo[1,3]dioxol-4-yl)-2-hydroxy-4-methyl-2-trifluoromethyl-pentanoic acid ethyl ester

4.42 g of 4-(benzo[1,3]dioxol-4-yl)-4-methyl-2-oxo-pentanoic acid ethyl ester and 6.9 ml of trifluoromethyl-trimethylsilane in 95 ml of THF are slowly mixed at −70° C. with 3.2 ml of TBAF solution (1H in THF). It is stirred for 1 hour at −70° C. and for 2 hours at room temperature, and a spatula tip full of solid tetrabutylammonium fluoride (TBAF) is added. After 1 hour of stirring, it is added to 0.1 N hydrochloric acid and extracted with ethyl acetate. The organic phase is washed with water and brine, dried (Na₂SO₄) and concentrated by evaporation. After chromatography on silica gel (hexane/ethyl acetate 0->10%), 4.55 g of the title compound is obtained as a yellow oil.

¹H-NMR (CDCl₃), □ (ppm)=1.19 (t, 3H), 1.39 (s, 3H), 1.46 (s, 3H), 2.29 (d, 1H), 2.74 (d, 1H), 3.59 (dq, 1H), 4.05 (dq, 1H), 5.92 (s, 1H), 5.98 (s, 1H), 6.68-6.85 (m, 3H)

4-(Benzo[1,3]dioxol-4-yl)-2-hydroxy-4-methyl-2-trifluoromethyl-pentanol

2.92 g of 4-(benzo[1,3]dioxol-4-yl)-2-hydroxy-4-methyl-2-trifluoromethyl-pentanoic acid ethyl ester in 100 ml of diethyl ether is mixed in portions at 0° C. with 478 mg of lithium aluminum hydride. After stirring for 10 hours, it is added to saturated bicarbonate solution and filtered through diatomaceous earth. The phases are separated, and the aqueous phase is extracted with ethyl acetate. The organic phase is washed with water and brine, dried (Na₂SO₄) and concentrated by evaporation. After chromatography on silica gel (hexane/ethyl acetate 0->10%), 2.44 g of the title compound is obtained as a yellow oil.

¹H-NMR (CDCl₃), □ (ppm)=1.42 (s, 3H), 1.51 (s, 3H), 2.22 (d, 1H), 2.36 (d, 1H), 2.9 (bs, 1H), 3.41 (d, 1H), 3.51 (d, 1H), 5.92 (s, 1H), 5.95 (s, 1H), 6.69-6.85 (m, 3H)

4-(Benzo[1,3]dioxol-4-yl)-2-hydroxy-4-methyl-2-trifluoromethyl-pentanal

0.650 ml of oxalyl chloride in 16.0 ml of dichloromethane is mixed at −78° C. with 1.2 ml of DMSO in 3.0 ml of dichloromethane. After 5 minutes, 2 g of 4-(benzo[1,3]dioxol-4-yl)-2-hydroxy-4-methyl-2-trifluoromethyl-pentanol in 7.0 ml of dichloromethane is added in drops at −78° C. After 15 minutes, it is mixed with 4.6 ml of triethylamine and slowly heated to room temperature. It is washed with water and brine, dried with sodium sulfate and concentrated by evaporation in a vacuum. After chromatography on silica gel (hexane/ethyl acetate 0->5%), 1.64 g of the title compound is obtained as a yellow oil.

¹H-NMR (CDCl₃), □ (ppm)=1.40 (s, 3H), 1.44 (s, 3H), 2.24 (d, 1H), 3.1 (d, 1H), 3.64 (bs, 1H), 5.94 (s, 1H), 5.99 (s, 1H), 6.67-6.9 (m, 3H), 9.05 (s, 1H)

Analogously to the production of Example 2, the corresponding imine is obtained from 125 mg of 5-amino-2-methylquinazoline and 237 mg of 4-(benzo[1,3]dioxol-4-yl)-2-hydroxy-4-methyl-2-trifluoromethyl-pentanal and reduced with palladium on activated carbon. The reoxidation is achieved by heating the product in xylene in the presence of palladium on activated carbon in the air. 61 mg of the title compound is obtained. ¹H-NMR (CDCl₃), □ (ppm)=1.48 (s, 3H), 1.56 (s, 3H), 2.35 (d, 1H), 2.53 (d, 1H), 2.85 (s, 3H), 3.23 (dd, 1H), 3.41 (dd, 1H), 4.74 (bt, 1H), 5.90 (s, 1H), 5.93 (s, 1H), 6.17 (d, 1H), 6.78 (dd, 1H), 6.84-6.93 (m, 2H), 7.28 (d, 1H), 7.58 (t, 1H), 9.21 (s, 1H)

### Example 33

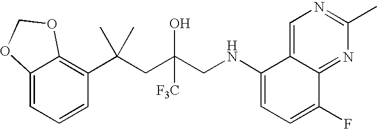

### 4-(Benzo[1,3]dioxol-4-yl)-4-methyl-1-(8-fluoro-2-methylquinazolin-5-ylamino)-2-(trifluoromethyl)-pentan-2-ol

Analogously to Example 25, 90 mg of 4-(benzo[1,3]dioxol-4-yl)-2-hydroxy-4-methyl-2-(trifluoromethyl)pentanal and 50 mg of 5-amino-8-fluoro-2-methylquinazoline in 10 ml of toluene are reacted. After chromatography on silica gel with hexane-ethyl acetate (0-70%), 58 mg of 4-(benzo[1,3]dioxol-4-yl)-4-methyl-1-(2-methylquinazolin -5-ylimino)-2-(trifluoromethyl)-pentan-2-ol is obtained. 20 mg of palladium on carbon is added to the imine in 10 ml of ethyl acetate and 1 ml of triethylamine, and it is shaken for 2 hours under a hydrogen atmosphere at normal pressure. Catalyst is removed from the solution by means of filtration, and it is concentrated by evaporation. It is taken up in 5 ml of chloroform, and 200 mg of activated manganese dioxide is added and stirred for 10 minutes. It is filtered on Celite and concentrated by evaporation in a vacuum. After chromatography on silica gel with hexane-ethyl acetate (0-70%), 15 mg of the product is obtained. ¹H-NMR (CDCl₃); δ=1.26 (s, 3H), 1.48 (s, 3H), 2.34 (d, 1H), 2.54 (d, 1H), 2.91 (s, 3H), 3.19 (dd, 1H), 3.34 (dd, 1H), 4.58 (br., 1H), 5.89 (d, 1H), 5.94 (d, 1H), 6.05 (dd, 1H), 6.71-6.85 (m, 3H), 7.32 (dd, 1H), 9.27 (s, 1H).

### Example 34

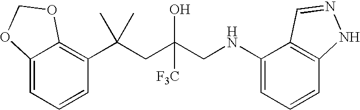

### 4-(Benzo[1,3]dioxol-4-yl)-4-methyl-1-(1H-indazol-4-ylamino)-2-(trifluoromethyl) -pentan-2-ol

Analogously to the production of Example 30, the corresponding imine that consists of 168 mg of 4-amino-1H-indazole and 383 mg of 4-(benzo[1,3]dioxol-4-yl)-2-hydroxy-4-methyl-2-trifluoromethyl-pentanal is obtained and further reduced with 488 mg of sodium cyanoborohydride to 240 mg of the title compound. ¹H-NMR (CDCl₃), □ (ppm)=1.44 (s, 3H), 1.58 (s, 3H), 2.27 (d, 1H), 2.54 (d, 1H), 3.22 (dd, 1H), 3.41 (dd, 1H), 4.01-4.17 (m, 1H), 5.81 (d, 1H), 5.92 s, 2H), 6.73-6.99 (m, 4H), 7.12 (t, 1H), 7.88 (s, 1H)

### Examples 35 and 36

### (−)-4-(Benzo[1,3]dioxol-4-yl)-4-methyl-1-(1H-indazol-4-ylamino)-2-(trifluoromethyl) -pentan-2-ol and (+)-4-(Benzo[1,3]dioxol-4-yl)-4-methyl-1-(1H-indazol-4-ylamino)-2-(trifluoromethyl)-pentan-2-ol

Separation of (+/−)-4-(benzo[1,3]dioxol-4-yl)-4-methyl-1-(1H-indazol-4-ylamino)-2-(trifluoromethyl)-pentan-2-ol:

The enantiomer mixture is separated by chromatography on chiral carrier material (CHIRALPAK AD®, DAICEL Company) with hexane/ethanol (90:10, vvv). Thus obtained are

(−)-enantiomer: MS (esi): M⁺+1=422, [□]_{D}−50.9° ^{o}(c=1.0, CHCl₃) and

(+)-enantiomer: MS (esi): M⁺+1=422, [□]_{D}+54.3° ^{o}(c=1.0, CHCl₃)

### Example 37

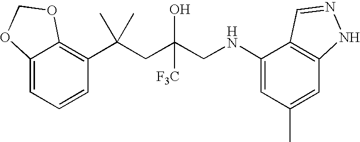

### 4-(Benzo[1,3]dioxol-4-yl)-1-(6-methyl-1H-indazol-4-ylamino)-4-methyl-2-(trifluoromethyl)pentan-2-ol

Analogously to Example 16, the desired product is obtained from 4-(benzo[1,3]dioxol-4-yl)-2-hydroxy-4-methyl-2-(trifluoromethyl)pentanal and 6-methyl-1H-indazol-4-ylamine (from Auwers Chem. Ber., 1920, 53, 1213). ¹H-NMR (CD₃OD); δ=1.44 (s, 3H), 1.65 (s, 3H), 2.06 (d, 1H), 2.23 (s, 3H), 2.8 (d, 2H), 3.09 (d, 1H), 5.89 (s, 1H), 5.91 (s, 1H), 6.05 (s, 1H), 6.75 (dd, 1H), 6.86 (t, 1H), 6.94 (dd, 1H), 7.22 (s, 1H), 7.73 (s, 1H).

### Example 38

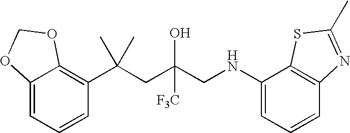

### 4-(Benzo[1,3]dioxol-4-yl)-4-methyl-1-(2-methyl-benzothiazol-7-ylamino)-2-(trifluoromethyl)-pentan-2-ol

Analogously to the production of Example 30, the corresponding imine is obtained from 100 mg of 7-amino-2-methylbenzothiazole (Libeer et al. Bull. Soc. Chim. Belg.; 1971; 80; 43-47) and 154 mg of 4-(benzo[1,3]dioxol-4-yl)-2-hydroxy-4-methyl-2-trifluoromethyl-pentanal, and further reduced with 63 mg of sodium cyanoborohydride to 8 mg of the title compound. ¹H-NMR (CDCl₃), □ (ppm)=1.42 (s, 3H), 1.53 (s, 3H), 2.01 (d, 1H), 2.10 (d, 1H), 2.78 (s, 3H), 5.29 (d, 1H), 5.67 (d, 1H), 6.00 (s, 2H), 6.13 (s, 1H), 6.63-6.82 (m, 3H), 7.18-7.30 (m, 2H)

### Example 39

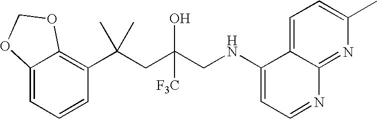

### 4-(Benzo[1,3]dioxol-4-yl)-4-methyl-1-(2-methyl-1,8-naphthyridin-5-ylamino)-2-(trifluoromethyl)-pentan-2-ol

Analogously to Example 10, 1-amino-4-(benzo[1,3]dioxol-4-yl)-4-methyl-2-(trifluoromethyl)propan-2-ol is reacted with 5-chloro-2-methyl-1,8-naphthyridine to form the desired product. ¹NMR (CDCl₃) □ (ppm)=1.49 (s, 3H), 1.57 (s, 3H), 2.41 (d, 1H), 2.49 (d, 1H), 2.74 (s, 3H), 3.32 (d, 1H), 3.50 (d, 1H), 5.89 (s, 1H), 5.93 (s, 1H), 6.06 (d, 1H), 6.76-6.92 (m, 3H), 7.23 (d, 1H), 8.05 (d, 1H), 8.61 (d, 1H)

### Example 40

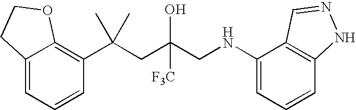

### 4-(2,3-Dihydrobenzofuranyl)-4-methyl-1-(1H-indazol-4-ylamino)-2-(trifluoromethyl)-pentan-2-ol

4-(2,3-Dihydrobenzofuranyl)-2-hydroxy-4-methyl-2-(trifluoromethyl)pentanal

Analogously to the described method of synthesis of 4-(benzo[1,3]dioxol-4-yl)-2-hydroxy-4-methyl-2-trifluoromethyl-pentanol (Example 32), starting from 513 mg of 4-(2,3-dihydrobenzofuranyl)-4-methyl-2-oxo-pentanoic acid ethyl ester (WO 00/32584), 142 mg of 4-(2,3-dihydrobenzofuranyl)-2-hydroxy-4-methyl-2-(trifluoromethyl)-pentanol as fraction 2 and 84 mg of the title compound as fraction 1 are obtained as colorless oils.

Fraction 1: ¹H-NMR (CDCl₃), □ (ppm)=1.39 (s, 3H), 1.45 (s, 3H), 2.18 (d, 1H), 3.19 (t, 2H), 3.34 (d, 1H), 3.62 (bs, 1H), 4.51-4.63 (m, 2H), 6.8 (t, 1H), 6.91 (d, 1H), 7.11 (d, 1H), 8.96 (s, 1H)

Fraction 2: ¹H-NMR (CDCl₃), □ (ppm)=1.41 (s, 3H), 1.48 (s, 3H), 2.25 (d, 1H), 2.48 (d, 1H), 3.2 (t, 2H), 3.42 (bs, 1H), 4.56 (t, 2H), 6.85 (t, 1H), 7.06-7.15 (m, 2H)

Analogously to the production of Example 30, the corresponding imine is obtained from 32 mg of 4-amino-1H-indazole and 75 mg of 4-(2,3-dihydrobenzofuranyl)-2-hydroxy-4-methyl-2-(trifluoromethyl)pentanal and further reduced with 134 mg of sodium cyanoborohydride to 64 mg of the title compound. ¹H-NMR (CDCl₃), □ (ppm)=1.45 (s, 3H), 1.57 (s, 3H), 2.31 (d, 1H), 2.64 (d, 1H), 3.05-3.19 (m, 2H), 3.21 (d, 1H), 3.39 (d, 1H), 4.48-4.63 (m, 2H), 5.76 (d, 1H), 6.85 (d, 1H), 6.92 (t, 1H), 7.05-7.23 (m, 3H), 7.85 (s, 1H)

### Example 41

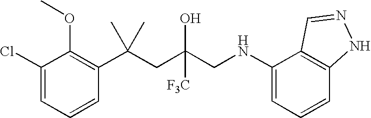

### 4-(3-Chloro-2-methoxyphenyl)-1-(1H-indazol-4-ylamino)-4-methyl-2-(trifluoromethyl)pentan-2-ol

3-Chloro-2-methoxybenzylcyanide

39.4 g (221.3 mmol) of NBS and 100 mg of benzoyl peroxide are added to 31.6 g (201.7 mmol) of 3-chloro-2-methoxytoluene in 500 ml of CCl₄. It is refluxed for 16 hours, allowed to cool, and filtered. Solvent is removed from the filtrate and dissolved in 214 ml of dimethylformamide and 142 ml of water. 20.9 g (322.1 mmol) of potassium cyanide is added at 0° C. and stirred over 16 hours. The reaction mixture is diluted with water and extracted several times with tert-butyl-methyl ether. The organic phase is washed several times with saturated sodium chloride solution and dried on sodium sulfate. The solvent is removed in a vacuum, and after chromatographic purification on silica gel (hexane/ethyl acetate 20%), 29.7 g of product is obtained. ¹H-NMR (CDCl₃): δ=3.76 (s, 2H), 3.95 (s, 3H), 7.08 (t, 1H), 7.31 (d, 1H), 7.37 (d, 1H)

4-(3-Chloro-2-methoxy-phenyl)-4-methyl-2-trifluoromethyl-pentane-1,2-diol

29.7 g (163.7 mmol) of 4-chloro-2-methoxybenzylcyanide and 46.5 g (327.4 mmol) of methyl iodide in 260 ml of DMF are mixed in portions at 0° C. with 13.2 g (327.4 mmol) of sodium hydride (60% in oil). It is stirred overnight and then mixed with water and ethyl acetate. The phases are separated, and the aqueous phase is extracted several times with ethyl acetate. It is washed with water and saturated sodium chloride solution, dried with sodium sulfate and concentrated by evaporation in a vacuum. After chromatography on silica gel (hexane/ethyl acetate 95:5), 32.4 g of 2-(4-chloro-2-methoxy-phenyl)-2-methylpropionitrile is obtained as a colorless oil. 7 g (33.4 mmol) of nitrile is slowly mixed in toluene at −78° C. with 41.6 ml (50.1 mmol) of diisobutylaluminum hydride solution (20% in toluene), and 5.55 ml of isopropanol is added in drops after 3 hours at −78° C. It is allowed to heat to −5° C., and 380 ml of a 10% aqueous tartaric acid solution is added. After dilution with ether, it is stirred vigorously, the organic phase is separated, and the aqueous phase is extracted several times with ether. It is washed with brine, dried with sodium sulfate and concentrated by evaporation in a vacuum. After chromatography on silica gel (hexane/ethyl acetate 95:5), 7.1 g of 2-(4-chloro-methoxy-phenyl)-2-methylpropanal is obtained as a colorless oil. A solution of 8.95 g (33.4 mmol) of 2-diethylphosphono-2-ethoxyacetic acid ethyl ester in 30 ml of tetrahydrofuran is mixed with 19 ml (38 mmol) of a 2 M solution of lithium diisopropylamide in tetrahydrofuran-heptane-toluene while being cooled with ice within 20 minutes, and it is stirred for 15 minutes at 0° C. A solution of 7.1 g (33.4 mmol) of 2-(3-chloro-2-methoxyphenyl)-2-methylpropanal in 27 ml of tetrahydrofuran is added in drops at 0° C. within 30 minutes. After 20 hours at room temperature, water is added, and it is extracted several times with ether and ethyl acetate. It is washed with saturated ammonium chloride solution, dried (Na₂SO₄) and concentrated by evaporation. The crude product is purified by column chromatography on silica gel (hexane/ethyl acetate 10%), and 8.5 g of 4-(3-chloro-2-methoxy-phenyl)-4-methyl-3-ethoxy-2-ene-valeric acid ethyl ester is obtained. The intermediate product is saponified with 80 ml of 3 M sodium hydroxide solution/160 ml of ethanol. 5.3 g of acid, which is stirred with 80 ml of 2N sulfuric acid at 90° C. over 16 hours, is obtained. After cooling, it is made basic with potassium carbonate, washed with ether and acidified with hydrochloric acid. After extraction with ethyl acetate, washing with saturated sodium chloride solution and removal of the solvent, 4.0 g of 4-(3-chloro-2-methoxyphenyl)-4-methyl-2-oxo-valeric acid is obtained. 6.6 g (24.3 mmol) of 4-(3-chloro-2-methoxy-phenyl)-4-methyl-2-oxo-valeric acid and 2.74 ml (51.4 mmol) of sulfuric acid (96%) are refluxed in 150 ml of ethanol for 5 hours. The batch is concentrated by evaporation in a vacuum, and the residue is taken up in saturated sodium bicarbonate solution. It is extracted several times with ethyl acetate, washed with saturated sodium bicarbonate solution, dried (sodium sulfate) and concentrated by evaporation in a vacuum. After chromatographic purification on silica gel (hexane/ethyl acetate 10%), 5.9 g of 4-(3-chloro-2-methoxy-phenyl)-4-methyl-2-oxo-valeric acid-ethyl ester is obtained. This ester and 3.4 g (23.8 mmol) of (trifluoromethyl)-trimethylsilane in 34 ml of THF are mixed with 49 mg of tetrabutylammonium fluoride at 0° C. It is stirred for 16 hours at room temperature and then the reaction mixture is added to water. It is extracted several times with ethyl acetate, washed with saturated sodium chloride solution, dried with sodium sulfate and concentrated by evaporation in a vacuum. 2.96 g of 4-(3-chloro-2-methoxy-phenyl)-2-hydroxy-4-methyl-2-trifluoromethyl-valeric acid-ethyl ester is obtained as a yellow oil. This oil is mixed in 24 ml of diethyl ether at 0° C. with 510 mg of lithium aluminum hydride and stirred for 4 more hours at room temperature. 20 ml of saturated sodium bicarbonate solution is carefully added at 0° C. to the batch, and it is vigorously stirred for 1 more hour. It is extracted several times with tert-butyl methyl ether, washed with water and saturated sodium chloride solution, dried with sodium sulfate and concentrated by evaporation in a vacuum. The crude product is mixed in 33 ml of THF with 1.83 (5.79 mmol) of tetrabutylammonium fluoride trihydrate and stirred for 16 hours. It is poured into ice water, extracted several times with tert-butyl methyl ether, washed with saturated sodium chloride solution, dried with sodium sulfate and concentrated by evaporation in a vacuum. After chromatographic purification on silica gel (hexane/ethyl acetate 25%), 1.81 g of 4-(3-chloro-2-methoxy-phenyl)-4-methyl-2-trifluoromethyl-pentane-1,2-diol is obtained. ¹H-NMR (CDCl₃), □ (ppm)=1.47 (s, 3H), 1.56 (s, 3H), 2.21 (d, 1H), 2.54 (d, 1H), 2.91 (s, 1H), 3.31 (dd, 1H), 3.42 (d, 1H), 4.01(s, 3H), 7.00 (t, 1H), 7.20-7.35 (m, 2H)

4-(3-Chloro-2-methoxy-phenyl)-2-hydroxy-4-methyl-2-trifluoromethyl-pentanal

1.87 g (18.5 mmol) of triethylamine and, in portions over 10 minutes, 1.17 g (7.4 mmol) of pyridine SO₃ complex are added to 1.2 g (3.7 mmol) of diol in 24 ml of dichloromethane and 6.4 ml of DMSO. It is stirred over 5 hours, and 30 ml of saturated ammonium chloride solution is added. The mixture is stirred for another 15 minutes, the phases are separated, and it is extracted with tert.-butyl ethyl ether. It is washed with water and dried on sodium sulfate. The solvent is removed in a vacuum, and after chromatographic purification on silica gel (hexane/ethyl acetate, 0-50%), 0.98 g of product is obtained. ¹H-NMR (CDCl₃): δ=1.44 (s, 3H), 1.50 (s, 3H), 2.29 (d, 2H), 3.28 (d, 1H), 3.55 (s, 1H), 4.01 (s, 3H), 6.95 (t, 1H), 7.07 (dd, 1H), 7.30 (dd, 1H), 8.90 (s, 1H).

Analogously to Example 16, the desired product is obtained from 4-(3-chloro-2-methoxyphenyl)-2-hydroxy-4-methyl-2-(trifluoromethyl)pentanal and 1H-indazol-4-ylamine.

¹H-NMR (300 MHz, CD₃OD): □=1.49 (3H), 1.68 (3H), 2.03 (1H), 3.00 (1H), 3.25 (1H), 3.35 (1H), 3.95 (3H), 5.60 (1H), 6.73-6.83 (2H), 7.03 (1H), 7.18 (1H), 7.30 (1H), 7.95 (1H).

### Example 42

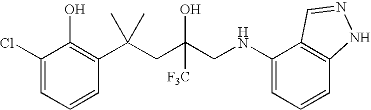

### 4-(3-Chloro-2-hydroxyphenyl)-1-(1H-indazol-4-ylamino)-4-methyl-2-(trifluoromethyl)pentan-2-ol

Analogously to Example 3, the desired product is obtained from 4-(3-chloro-2-methoxyphenyl)-1-(1H-indazol-4-ylamino)-4-methyl-2-(trifluoromethyl)pentan-2-ol. ¹H-NMR (300 MHz, CD₃OD): □=1.50 (3H), 1.70 (3H), 2.03 (1H), 3.00 (1H), 3.25 (1H), 3.35 (1H), 5.60 (1H), 6.73-6.83 (2H), 7.03 (1H), 7.18 (1H), 7.30 (1H), 7.95 (1H).

### Example 43

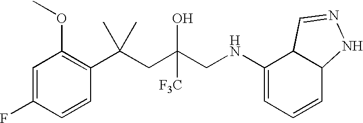

### 4-(4-Fluoro-2-methoxyphenyl)-4-methyl-1-(1H-indazol-4-ylamino)-2-(trifluoromethyl) -pentan-2-ol

4-(4-Fluoro-2-methoxyphenyl)-2-hydroxy-4-methyl-2-(trifluoromethyl)pentanol and 4-(2-fluoro-4-methoxyphenyl)-2-hydroxy-4-methyl-2-(trifluoromethyl)pentanol

Both products are obtained analogously to the method of synthesis of 4-(4-chloro-2-methoxyphenyl)-2-hydroxy-4-methyl-2-(trifluoromethyl)pentanol and 4-(2-chloro-4-methoxyphenyl)-2-hydroxy-4-methyl-2-(trifluoromethyl)pentanol (Example 45) from 3-fluoroanisole and 2-hydroxy-4-methylene-2-(trifluoromethyl)valeric acid ethyl ester and the subsequent reaction with lithium aluminum hydride. The separation is carried out by chromatography on silica gel.

1st Fraction: ¹H-NMR (CDCl₃), □ (ppm)=1.40 (s, 3H), 1.52 (s, 3H), 2.25 (d, 1H), 2.50 (d, 1H), 2.83 (bs, 1H), 3.36 (d, 1H), 3.46 (d, 1H), 3.85 (s, 3H), 6.54-6.69 (m, 2H), 7.19-7.30 (m, 1H)

2nd Fraction: ¹H-NMR (CDCl₃), □ (ppm)=1.41 (s, 3H), 1.53 (s, 3H), 2.17 (d, 1H), 2.3 (d, 1H), 2.89 (bs, 1H), 3.34 (bd, 1H), 3.53 (d, 1H), 3.78 (s, 3H), 6.58 (dd, 1H), 6.63 (dd, 1H), 7.22 (t, 1H)

4-(4-Fluoro-2-methoxyphenyl)-2-hydroxy-4-methyl-2-(trifluoromethyl)pentanal

Analogously to the synthesis of 4-(4-chloro-2-methoxyphenyl)-2-hydroxy-4-methyl-2-(trifluoromethyl)pentanal, 1.81 g of the title compound is obtained as a cloudy oil starting from 3 g of 4-(4-fluoro-2-methoxyphenyl)-2-hydroxy-4-methyl-2-(trifluoromethyl)pentanol.

¹H-NMR (CDCl₃), □ (ppm)=1.38 (s, 3H), 1.46 (s, 3H), 2.19 (d, 1H), 3.37 (d, 1H), 3.58 (s, 1H), 3.87 (s, 3H), 6.55-6.64 (m, 2H), 7.06 (dd, 1H), 8.97 (s, 1H)

Analogously to the production of Example 30, the corresponding imine is obtained from 64 mg of 4-amino-1H-indazole and 150 mg of 4-(4-fluoro-2-methoxyphenyl)-2-hydroxy-4-methyl-2-(trifluoromethyl)pentanal and further reduced with 141 mg of sodium cyanoborohydride to 53 mg of the title compound. ¹H-NMR (CDCl₃), □ (ppm)=1.44 (s, 3H), 1.59 (s, 3H), 2.29 (d, 1H), 2.70 (d, 1H), 3.18 (d, 1H), 3.33 (d, 1H), 3.84 (s, 3H), 5.64 (d, 1H), 6.65 (dd, 1H), 6.71 (dt, 1H), 6.88 (d, 1H), 7.12 (t, 1H), 7.37 (dd, 1H), 7.90 (s, 1H)

### Example 44

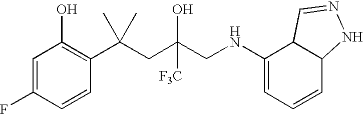

### 4-(4-Fluoro-2-hydroxyphenyl)-4-methyl-1-(1H-indazol-4-ylamino)-2-(trifluoromethyl) -pentan-2-ol

Analogously to the production of Example 3, 4 mg of the title compound is obtained from the reaction of 45 mg of 4-(4-fluoro-2-methoxyphenyl)-4-methyl-1-(1H-indazol-4-ylamino)-2-(trifluoromethyl)-pentan-2-ol and 1.65 ml of boron tribromide (1 M in dichloromethane). ¹H-NMR (CDCl₃), □ (ppm)=1.48 (s, 3H), 1.52 (s, 3H), 2.26 (d, 1H), 2.84 (d, 1H), 3.25 (d, 1H), 3.41 (d, 1H), 4.23 (bs, 1H), 5.72 (d, 1H), 6.44 (dd, 1H), 6.64 (dt, 1H), 6.83 (d, 1H), 7.12 (t, 1H), 7.33 (dd, 1H), 7.96 (s, 1H)

### Example 45

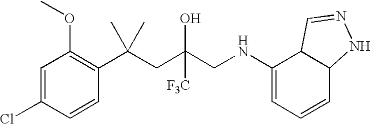

### 4-(4-Chloro-2-methoxyphenyl)-4-methyl-1-(1H-indazol-4-ylamino)-2-(trifluoromethyl) -pentan-2-ol

4-(4-Chloro-2-methoxyphenyl)-2-hydroxy-4-methyl-2-(trifluoromethyl)pentanol and 4-(2-chloro-4-methoxyphenyl)-2-hydroxy-4-methyl-2-(trifluoromethyl)pentanol

A solution of 3 g of 2-hydroxy-4-methylene-2-(trifluoromethyl)valeric acid ethyl ester in 22 ml of 3-chloroanisole is mixed in portions with aluminum trichloride at room temperature. It is stirred for 48 hours and then mixed with 2N hydrochloric acid and hexane and stirred for 1 hour. It is washed with 2N hydrochloric acid and water, and excess 3-chloroanisole is distilled off in a vacuum. The remaining residue is purified by chromatography on silica gel (hexane/ethyl acetate 0-10%). 2.85 g of a mixture of 4-(4-chloro-2-methoxyphenyl)-2-hydroxy-4-methy-2-(trifluoromethyl)valeric acid ethyl ester and 4-(2-chloro-4-methoxyphenyl)-2-hydroxy-4-methy-2-(trifluoromethyl)valeric acid ethyl ester is obtained as a yellow oil. This substance mixture is mixed in 90 ml of ether at 0° C. with 445 mg of lithium aluminum hydride and stirred for 12 hours. The batch is added to saturated sodium bicarbonate solution, filtered through diatomaceous earth, the phases are separated, and the aqueous phase is extracted with ethyl acetate. It is washed with water and brine, dried with sodium sulfate, and concentrated by evaporation in a vacuum. After chromatography on silica gel (hexane/ethyl acetate 95:5), 1.87 mg of 4-(4-chloro-2-methoxyphenyl)-2-hydroxy-4-methyl-2-(trifluoromethyl)pentan-1-ol as a 1^{st} fraction and 160 mg of 4-(2-chloro-4-methoxyphenyl)-2-hydroxy-4-methyl-2-(trifluoromethyl)pentan-1-ol as a 2^{nd} fraction are obtained as colorless oils.

1st Fraction: ¹H-NMR (CDCl₃), □ (ppm)=1.41 (s, 3H), 1.51 (s, 3H), 2.24 (d, 1H), 2.51 (d, 1H), 2.84 (bs, 1H), 3.36 (d, 1H), 3.48 (d, 1H), 3.85 (s, 3H), 6.88 (d, 1H), 6.92 (dd, 1H), 7.24 (d, 1H)

2nd Fraction: ¹H-NMR (CDCl₃), □ (ppm)=1.52 (s, 3H), 1.62 (s, 3H), 2.18 (d, 1H), 2.76 (d, 1H), 2.93 (bs, 1H), 3.33 (d, 1H), 3.55 (d, 1H), 3.80 (s, 3H), 6.78 (dd, 1H), 6.90 (d, 1H), 7.38 (d, 1H)

4-(4-Chloro-2-methoxyphenyl)-2-hydroxy-4-methyl-2-(trifluoromethyl)pentanal

0.425 ml of oxalyl chloride in 10 ml of dichloromethane is mixed at −78° C. with 0.77 ml of DMSO in 2.0 ml of dichloromethane. After 5 minutes, 1.38 g of 4-(4-chloro-2-methoxyphenyl)-2-hydroxy-4-methyl-2-(trifluoromethyl)pentan-1-ol in 6.0 ml of dichloromethane is added in drops at −78° C. After 15 minutes, it is mixed with 2.9 ml of triethylamine and slowly heated to room temperature. It is washed with water and brine, dried with sodium sulfate and concentrated by evaporation in a vacuum. After chromatography on silica gel (hexane/ethyl acetate 98:2), 1.16 g of 4-(4-chloro-2-methoxyphenyl)-2-hydroxy-4-methyl-2-(trifluoromethyl)-pentanal is obtained as a colorless oil. ¹H-NMR (CDCl₃), □ (ppm)=1.38 (s, 3H), 1.44 (s, 3H), 2.21 (d, 1H), 3.34 (d, 1H), 3.57 (bs, 1H), 3.89 (s, 3H), 6.84 (d, 1H), 6.87 (d, 1H), 7.04 (d, 1H), 9.02 (s, 1H)

Analogously to the production of 4-(5-fluoro-2-methoxyphenyl)-4-methyl-1-(2-methylphthalazin-1-on-5-ylamino)-2-(trifluoromethyl)-pentan-2-ol (Example 27), the corresponding imine is obtained from 168 mg of 4-amino-1H-indazole and 410 mg of 4-(4-chloro-2-methoxyphenyl)-2-hydroxy-4-methyl-2-(trifluoromethyl)pentanal and further reduced with 580 mg of sodium cyanoborohydride to 303 mg of the title compound. ¹H-NMR (CDCl₃), □ (ppm)=1.44 (s, 3H), 1.59 (s, 3H), 2.26 (d, 1H), 2.70 (d, 1H), 3.17 (dd, 1H), 3.31 (dd, 1H), 3.85 (s, 3H), 4.08 (bs, 1H), 5.53 (d, 1H), 6.86 (d, 1H), 6.89 (d, 1H), 6.99 (dd, 1H), 7.14 (t, 1H), 7.34 (d, 1H), 7.86 (s, 1H)

### Examples 46 and 47

### (−)-4-(4-Chloro-2-methoxyphenyl)-4-methyl-1-(1H-indazol-4-ylamino)-2-(trifluoromethyl)-pentan-2-ol and (+)-4-(4-Chloro-2-methoxyphenyl)-4-methyl-1-(1H-indazol-4-ylamino)-2-(trifluoromethyl)-pentan-2-ol

Separation of (+/−)-4-(4-chloro-2-methoxyphenyl)-4-methyl-1-(1H-indazol-4-ylamino)-2-(trifluoromethyl)-pentan-2-ol:

The enantiomer mixture is separated by chromatography on chiral carrier material (CHIRALPAK AD®, DAICEL Company) with hexane/ethanol (80:20, vvv). Thus obtained are

(−)-Enantiomer: MS (esi): M⁺+1=442/444, [□]_{D}−60.8° ^{o}(c=1.0, CHCl₃) and

(+)-Enantiomer: MS (esi): M⁺+1=442/444, [□]_{D}+43.0° ^{o}(c=1.0, CHCl₃)

### Examples 48 and 49

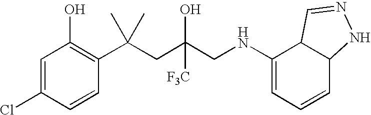

### (−)-4-(4-Chloro-2-hydroxyphenyl)-4-methyl-1-(1H-indazol-4-ylamino)-2-(trifluoromethyl)-pentan-2-ol

Analogously to the production of Example 3, 11 mg of the title compound is obtained from the reaction of 100 mg of (−)-4-(4-chloro-2-methoxyphenyl)-4-methyl-1-(1H-indazol-4-ylamino)-2-(trifluoromethyl)-pentan-2-ol and 3.6 ml of boron tribromide (1 M in dichloromethane).

¹H-NMR (CDCl₃), □ (ppm)=1.49 (s, 3H), 1.62 (s, 3H), 2.25 (d, 1H), 2.82 (d, 1H), 3.25 (d, 1H), 3.40 (d, 1H), 5.63 (d, 1H), 6.72 (d, 1H), 6.84 (d, 1H), 6.93 (d, 1H), 7.14 (t, 1H), 7.31 (d, 1H), 7.95 (s, 1H)

### (+)-4-(4-Chloro-2-hydroxyphenyl)-4-methyl-1-(1H-indazol-4-ylamino)-2-(trifluoromethyl)-pentan-2-ol

Analogously to the production of Example 3, 2.17 g of the title compound is obtained from the reaction of 5.3 g of (+)-4-(4-chloro-2-methoxyphenyl)-4-methyl-1-(1H-indazol-4-ylamino)-2-(trifluoromethyl)-pentan-2-ol and 190 ml of boron tribromide (1 M in dichloromethane).

¹H-NMR (CDCl₃), □ (ppm)=1.49 (s, 3H), 1.62 (s, 3H), 2.25 (d, 1H), 2.82 (d, 1H), 3.25 (d, 1H), 3.40 (d, 1H), 5.63 (d, 1H), 6.72 (d, 1H), 6.84 (d, 1H), 6.93 (d, 1H), 7.14 (t, 1H), 7.31 (d, 1H), 7.95 (s, 1H)

### Example 50

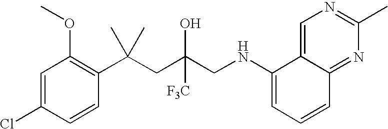

### 4-(4-Chloro-2-methoxyphenyl)-4-methyl-1-(2-methylquinazolin-5-ylamino)-2-(trifluoromethyl)-pentan-2-ol

Analogously to the production of Example 2, the corresponding imine is obtained from 395 mg of 5-amino-2-methylquinazoline and 299 mg of 4-(4-chloro-2-methoxyphenyl)-2-hydroxy-4-methyl-2-(trifluoromethyl)pentanal, and it is reduced with palladium on activated carbon. The reoxidation is achieved by heating the product in xylene in the presence of palladium on activated carbon in air. 11 mg of the title compound is obtained. ¹H-NMR (CDCl₃), □ (ppm)=1.50 (s, 3H), 1.59 (s, 3H), 2.45 (d, 1H), 2.68 (d, 1H), 2.86 (s, 3H), 3.18 (dd, 1H), 3.36 (dd, 1H), 3.89 (s, 3H), 6.08 (d, 1H), 6.92 (d, 1H), 6.98 (t, 1H), 7.36 (d, 1H), 7.54 (d, 1H), 9.11 (s, 1H)

### Example 51

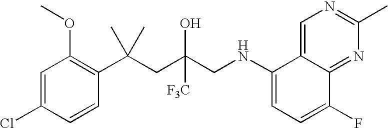

### 4-(4-Chloro-2-methoxyphenyl)-1-(8-fluoro-2-methylquinazolin-5-ylamino)-4-methyl-2-(trifluoromethyl)-pentan-2-ol

1.72 g (5.30 mmol) of 4-(4-chloro-2-methoxyphenyl)-2-hydroxy-4-methyl-2-trifluoromethyl-pentanal and 0.85 g (4.80 mmol) of 5-amino-8-fluoro-2-methyl-quinazoline are first reacted analogously to Example 2 in 64 ml of dichloromethane and 13 ml of acetic acid. 600 mg of purified intermediate product is obtained.

200 mg (0.42 mmol) of this imine is dissolved in 7 ml of THF, and 60 mg (0.84 mmol) of sodium cyanoborohydride is added at 0° C. After 1 hour, several drops of methanol/acetic acid (1:1) as well as another 15 mg (0.21 mmol) of sodium cyanoborohydride are added and stirred for another 2 hours. The reaction is brought to a halt by adding saturated ammonium chloride solution and worked up analogously to Example 26. The crude product is chromatographed on silica gel (eluant hexane/ethyl acetate 20%). This product is taken up in a little chloroform, mixed with a spatula tip full of manganese dioxide and stirred for 1 hour. The manganese dioxide is filtered off, the filtrate is concentrated by evaporation and chromatographed with a little silica gel with hexane/ethyl acetate 20-50%. 5 mg of the desired product is obtained. ¹H-NMR (CDCl₃), □ (ppm)=1.49 (s, 3H), 1.56 (s, 3H), 2.45 (d, 1H), 2.68 (d, 1H), 2.88 (s, 3H), 3.16 (dd, 1H), 3.30 (dd, 1H), 3.89 (s, 3H), 5.92 (dd, 1H), 6.85 (d, 1H), 6.92 (dd, 1H), 7.30 (dd, 1H), 7.36 (d, 1H), 9.19 (s, 1H); MS (CI): 486/488 (M+H).

### Example 52

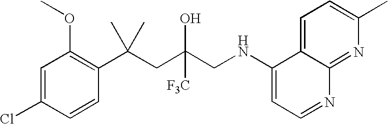

### 4-(4-Chloro-2-methoxyphenyl)-4-methyl-1-(2-methyl-1,8-naphthyridin-5-ylamino)-2-(trifluoromethyl)pentan-2-ol

Analogously to Example 10, 1-amino-4-(4-chloro-2-methoxyphenyl)-4-methyl-2-(trifluoromethyl)propan-2-ol is reacted with 5-chloro-2-methyl-1,8-naphthyridine to form the desired product. ¹H-NMR (CDCl₃); δ=1.46 (s, 3H), 1.56 (s, 3H), 2.46 (d, 1H), 2.62 (d, 1H), 2.70 (s, 3H), 3.22 (d, 1H), 3.38 (dd, 1H), 3.84 (s, 3H), 5.89 (d, 1H), 6.87 (d, 1H), 6.94 (dd, 1H), 7.14 (d, 1H), 7.27 (d, 1H), 7.82 (d, 1H), 8.58 (s, 1H).

### Example 53

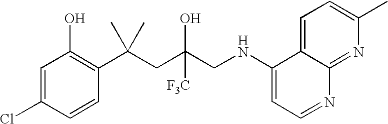

### 4-(4-Chloro-2-hydroxyphenyl)-4-methyl-1-(2-methyl-1,8-naphthyridin-5-ylamino)-2-(trifluoromethyl)-pentan-2-ol

Analogously to Example 3, 4-(4-chloro-2-methoxyphenyl)-4-methyl-1-(2-methyl-1,8-naphthyridin-5-ylamino)-2-(trifluoromethyl)pentan-2-ol is reacted to form the desired product. ¹H-NMR (CD₃OD); δ=1.47 (s, 3H), 1.66 (s, 3H), 2.02 (d, 1H), 2.18 (d, 1H), 2.72 (s, 3H), 3.14 (d, 1H), 3.24 (dd, 1H), 5.94 (d, 1H), 6.65 (d, 1H), 6.76 (dd, 1H), 7.31 (d, 1H), 7.40 (d, 1H), 8.26 (d, 1H), 8.35 (s, 1H).

### Example 54

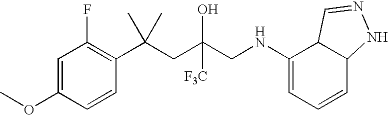

### 4-(2-Fluoro-4-methoxyphenyl)-4-methyl-1-(1H-indazol-4-ylamino)-2-(trifluoromethyl) -pentan-2-ol

4-(2-Fluoro-4-methoxyphenyl)-2-hydroxy-4-methyl-2-(trifluoromethyl)pentanal

Analogously to the synthesis of 4-(4-chloro-2-methoxyphenyl)-2-hydroxy-4-methyl-2-(trifluoromethyl)pentanal (Example 45), 1.73 g of the title compound is obtained as a colorless oil starting from 3 g of 4-(2-fluoro-4-methoxyphenyl)-2-hydroxy-4-methyl-2-(trifluoromethyl)pentanol (Example 47).

¹H-NMR (CDCl₃), □ (ppm)=1.39 (s, 3H), 1.46 (s, 3H), 2.26 (d, 1H), 3.09 (d, 1H), 3.63 (s, 1H), 3.78 (s, 3H), 6.52-6.65 (m, 2H), 7.03 (t, 1H), 9.04 (s, 1H)

Analogously to the production of Example 30, the corresponding imine is obtained from 82 mg of 4-amino-1H-indazole and 192 mg of 4-(2-fluoro-4-methoxyphenyl)-2-hydroxy-4-methyl-2-(trifluoromethyl)pentanal and further reduced with 281 mg of sodium cyaonoborohydride to 152 mg of the title compound. ¹H-NMR (CDCl₃), □ (ppm)=1.47 (s, 3H), 1.61 (s, 3H), 2.23 (d, 1H), 2.50 (d, 1H), 3.24 (dd, 1H), 3.37 (dd, 1H), 3.78 (s, 3H), 4.00-4.11 (m, 1H), 5.70 (d, 1H), 6.61 (dd, 1H), 6.72 (dd, 1H), 6.87 (d, 1H), 7.08 (t, 1H), 7.33 (t, 1H), 7.90 (s, 1H)

### Example 55

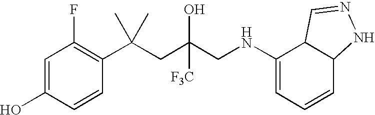

### 4-(2-Fluoro-4-hydroxyphenyl)-4-methyl-1-(1H-indazol-4-ylamino)-2-(trifluoromethyl) -pentan-2-ol

Analogously to the production of Example 3, 76 mg of the title compound is obtained from the reaction of 80 mg of 4-(2-fluoro-4-methoxyphenyl)-4-methyl-1-(1H-indazol-4-ylamino)-2-(trifluoromethyl)-pentan-2-ol and 3 ml of boron tribromide (1 M in dichloromethane). ¹H-NMR (CDCl₃), □ (ppm)=1.44 (s, 3H), 1.60 (s, 3H), 2.22 (d, 1H), 2.49 (d, 1H), 3.28 (d, 1H), 3.37 (d, 1H), 5.83 (d, 1H), 6.58 (dd, 1H), 6.66 (dd, 1H), 6.85 (d, 1H), 7.16 (t, 1H), 7.27 (d, 1H), 7.88 (s, 1H)

### Example 56

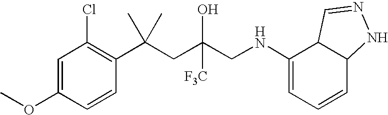

### 4-(2-Chloro-4-methoxyphenyl)-4-methyl-1-(1H-indazol-4-ylamino)-2-(trifluoromethyl) -pentan-2-ol

4-(2-Chloro-4-methoxyphenyl)-2-hydroxy-4-methyl-2-(trifluoromethyl)pentanal

Analogously to the synthesis of 4-(4-chloro-2-methoxyphenyl)-2-hydroxy-4-methyl-2-(trifluoromethyl)pentanal (Example 45), 101 mg of the title compound is obtained as a yellow oil starting from 150 mg of 4-(2-chloro-4-methoxyphenyl)-2-hydroxy-4-methyl-2-(trifluoromethyl)pentanol. ¹H-NMR (CDCl₃), □ (ppm)=1.52 (s, 3H), 1.54 (s, 3H), 2.24 (d, 1H), 3.60 (d, 1H), 3.65 (s, 1H), 3.79 (s, 3H), 6.72 (dd, 1H), 6.88 (d, 1H), 7.19 (d, 1H), 9.11 (s, 1H)

Analogously to the production of Example 30, the corresponding imine is obtained from 36 mg of 4-amino-1H-indazole and 90 mg of 4-(2-chloro-4-methoxyphenyl)-2-hydroxy-4-methyl-2-(trifluoromethyl)pentanal and further reduced to 54 mg of the title compound with 114 mg of sodium cyanoborohydride. ¹H-NMR (CDCl₃), □ (ppm)=1.56 (s, 1H), 1.71 (s, 3H), 2.19 (d, 1H), 3.01 (d, 1H), 3.28 (d, 1H), 3.37 (d, 1H), 3.79 (s, 3H), 5.56 (d, 1H), 6.83 (dd, 1H), 6.86 (d, 1H), 7.06 (t, 1H), 7.49 (d, 1H), 7.90 (s, 1H)

### Example 57

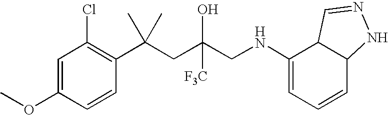

### 4-(2-Chloro-4-hydroxyphenyl)-4-methyl-1-(1H-indazol-4-ylamino)-2-(trifluoromethyl) -pentan-2-ol

Analogously to the production of Example 3, 11 mg of the title compound is obtained from the reaction of 27 mg of 4-(2-chloro-4-methoxyphenyl)-4-methyl-1-(1H-indazol-4-ylamino)-2-(trifluoromethyl)-pentan-2-ol and 0.93 ml of boron tribromide (1 M in dichloromethane). ¹H-NMR (CDCl₃), □ (ppm)=1.54 (s, 3H), 1.69 (s, 3H), 2.19 (d, 1H), 3.00 (d, 1H), 3.25-3.46 (m, 2H), 4.01 (bs, 1H), 5.75 (d, 1H), 6.77 (dd, 1H), 6.82 (d, 1H), 6.92 (d, 1H), 7.11 (t, 1H), 7.41 (d, 1H), 7.86 (s, 1H)

### Example 58

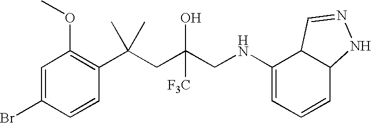

### 4-(4-Bromo-2-methoxyphenyl)-4-methyl-1-(1H-indazol-4-ylamino)-2-(trifluoromethyl) -pentan-2-ol

4-(4-Bromo-2-methoxyphenyl)-4-methyl-2-(trifluoromethyl)-pentane-1,2-diol

2.55 g (6.17 mmol) of 4-(4-bromo-2-methoxyphenyl)-2-hydroxy-4-methyl-2-(trifluoromethyl)-pentanoic acid ethyl ester (synthesized in two stages starting from 4-(4-bromo-2-methoxyphenyl)-2-oxopentanoic acid (WO 98/54159)) is dissolved in 102 ml of diethyl ether, mixed in portions at 0 to −5° C. with 351.3 mg (9.256 mmol) of lithium aluminum hydride and stirred for three and one-half hours at room temperature. The reaction mixture is mixed drop by drop with saturated sodium bicarbonate solution while being cooled in an ice bath, and stirred for 15 minutes at 5° C. and then for one hour at room temperature. The deposited precipitate is suctioned off, rewashed with diethyl ether, and the filtrate is concentrated by evaporation in a rotary evaporator. The residue is chromatographed on silica gel (mobile solvent ethyl acetate/hexane). In addition to 308 mg of aldehyde (see next stage), 2.025 g (88.4%) of the diol is obtained.

4-(4-Bromo-2-methoxyphenyl)-2-hydroxy-4-methyl-2-(trifluoromethyl)-pentanal

2.03 g (5.442 mmol) of 4-(4-bromo-2-methoxyphenyl)-4-methyl-2-(trifluoromethyl)-pentane-1,2-diol is oxidized to aldehyde according to Swern as described in Example 49. 1.839 g (91.4%) of the desired compound is isolated. ¹H-NMR (300 MHz, CDCl₃): □=1.39 (3H), 1.45 (3H), 2.23 (1H), 3.35 (1H), 3.58 (1H), 3.90 (3H), 6.93-7.09 (3H) 9.03 (1H).

Analogously to the production of Example 30, the corresponding imine is obtained from 181 mg of 4-amino-1H-indazole and 500 mg of 4-(4-bromo-2-methoxyphenyl)-2-hydroxy-4-methyl-2-(trifluoromethyl)pentanal and further reduced to 190 mg of the title compound with 334 mg of sodium cyanoborohydride. ¹H-NMR (CDCl₃), □ (ppm)=1.44 (s, 3H), 1.59 (s, 3H), 2.24 (d, 1H), 2.71 (d, 1H), 3.17 (dd, 1H), 3.31 (dd, 1H), 3.87 (s, 3H), 4.01-4.10 (m, 1H), 5.52 (d, 1H), 6.68 (d, 1H), 7.05 (d, 1H), 7.15 (dd, 1H), 7.15 (t, 1H), 7.27 (d, 1H), 7.88 (s, 1H)

### Example 59

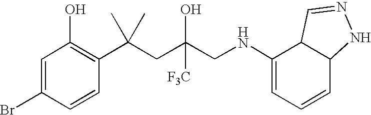

### 4-(4-Bromo-2-hydroxyphenyl)-4-methyl-1-(1H-indazol-4-ylamino)-2-(trifluoromethyl) -pentan-2-ol

Analogously to the production of Example 3, 14 mg of the title compound is obtained from the reaction of 90 mg of 4-(4-bromo-2-methoxyphenyl)-4-methyl-1-(1H-indazol-4-ylamino)-2-(trifluoromethyl)-pentan-2-ol and 3.8 ml of boron tribromide (1 M in dichloromethane). ¹H-NMR (CDCl₃), □ (ppm)=1.48 (s, 3H), 1.60 (s, 3H), 2.22 (d, 1H), 2.86 (d, 1H), 3.23 (d, 1H), 3.39 (d, 1H), 4.22 (bs, 1H), 5.65 (d, 1H), 6.83 (d, 1H), 6.85 (d, 1H), 7.04 (dd, 1H), 7.15 (t, 1H), 7.23 (d, 1H), 7.95 (s, 1H)

### Example 60

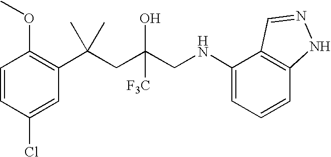

### 4-(5-Chloro-2-methoxyphenyl)-1-(1H-indazol-4-ylamino)-4-methyl-2-(trifluoromethyl)pentan-2-ol

4-(5-Chloro-2-methoxyphenyl)-2-hydroxy-4-methyl-2-(trifluoromethyl)pentanal

2 g (6.12 mmol) of 4-(5-chloro-2-methoxyphenyl)-4-methyl-2-trifluoromethyl-pentane-1,2-diol is oxidized with 854.6 mg (6.733 mmol) of oxalyl chloride and 1.05 ml (14.812 mmol) of DMSO according to Swern as described in Example 49. After the working-up, 1.95 g (98.4%) of the desired aldehyde, which is incorporated in crude form into the next stage, is obtained. ¹H-NMR (300 MHz, CDCl₃): □=1.39 (3H), 1.49 (3H), 2.27 (1H), 3.32 (1H), 3.59 (1H), 3.88 (3H), 6.78 (1H), 7.10 (1H), 7.20 (1H), 9.09 (1H).

Analogously to Example 16, the desired product is obtained from 4-(5-chloro-2-methoxyphenyl)-2-hydroxy-4-methyl-2-(trifluoromethyl)pentanal and 1H-indazol-4-ylamine.

¹H-NMR (300 MHz, CDCl₃): □=1.45 (3H), 1.60 (3H), 2.25 (1H), 2.78 (1H), 3.13 (1H), 3.35 (1H), 3.83 (3H), 5.60 (1H), 6.82 (1H), 6.87 (2H), 7.15 (1H), 7.25 (1H), 7.40 (1H), 7.86 (1H).

### Example 61

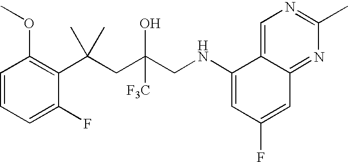

### 4-(6-Fluoro-2-methoxyoxyphenyl)-4-methyl-1-(7-fluoro-2-methylquinazolin-5-ylamino)-2-(trifluoromethyl)-pentan-2-ol

Analogously to Example 25, 4-(6-fluoro-2-methoxyphenyl)-2-hydroxy-4-methyl-2-trifluoromethyl-pentanal is reacted with 5-amino-7-fluoro-2-methylquinazoline to form the desired product. ¹H-NMR (CDCl₃); δ=1.59 (d, 3H), 1.69 (d, 3H), 2.35 (d, 1H), 2.65 (d, 1H), 2.80 (s, 3H), 3.21 (dd, 1H), 3.41 (dd, 1H), 3.86 (s, 3H), 5.02 (br., 1H), 5.89 (dd, 1H), 6.62 (dd, 1H), 6.67 (d, 1H), 6.83 (dd, 1H), 7.14 (ddd, 1H), 8.98 (s, 1H).

### Example 62

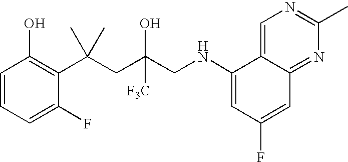

### 4-(6-Fluoro-2-hydroxyoxyphenyl)-1-(7-fluoro-2-methylquinazolin-5-ylamino)-4-methyl-2-(trifluoromethyl)pentan-2-ol

Analogously to Example 3, 4-(6-fluoro-2-methoxyphenyl)-4-methyl-1-(8-fluoro-2-methylquinazolin-5-ylamino)-2-(trifluoromethyl)-pentan-2-ol is reacted to form the desired product. ¹H-NMR (CDCl₃); δ=1.64 (d, 3H), 1.73 (d, 3H), 2.40 (d, 1H), 2.73 (d, 1H), 2.78 (s, 3H), 3.32 (dd, 1H), 3.49 (dd, 1H), 3.84 (s, 1H), 5.13 (br., 1H), 5.98 (dd, 1H), 6.48 (d, 1H), 6.54 (dd, 1H), 6.80 (dd, 1H), 6.93 (ddd, 1H), 8.97 (s, 1H).

### Example 63

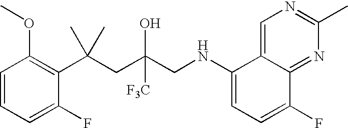

### 4-(6-Fluoro-2-methoxyphenyl)-4-methyl-1-(8-fluoro-2-methylquinazolin-5-ylamino)-2-(trifluoromethyl)-pentan-2-ol

Analogously to Example 25, 4-(6-fluoro-2-methoxyphenyl)-2-hydroxy-4-methyl-2-trifluoromethyl-pentanal is reacted with 5-amino-8-fluoro-2-methylquinazoline to form the desired product. ¹H-NMR (CDCl₃); δ=1.58 (d, 3H), 1.68 (d, 3H), 2.35 (d, 1H), 2.69 (d, 1H), 2.90 (s, 3H), 3.04 (s, 1H), 3.23 (dd, 1H), 3.40 (dd, 1H), 3.85 (s, 3H), 4.56 (br., 1H), 6.05 (dd, 1H), 6.58-6.68 (m, 2H), 7.14 (ddd, 1H), 7.30 (dd, 1H), 9.17 (s, 1H).

### Example 64

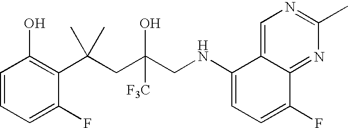

### 4-(6-Fluoro-2-hydroxyphenyl)-4-methyl-1-(8-fluoro-2-methylquinazolin-5-ylamino)-2-(trifluoromethyl)-pentan-2-ol

Analogously to Example 3, 4-(6-fluoro-2-methoxyphenyl)-4-methyl-1-(8-fluoro-2-methylquinazolin-5-ylamino)-2-(trifluoromethyl)-pentan-2-ol is reacted to form the desired product. ¹H-NMR (CDCl₃); δ=1.62 (d, 3H), 1.74 (d, 3H), 2.26 (d, 1H), 2.85 (d, 1H), 2.63 (s, 1H), 2.89 (s, 3H), 3.32 (dd, 1H), 3.44 (dd, 1H), 4.64 (br., 1H), 6.10 (dd, 1H), 6.46-6.58 (m, 2H), 6.93 (ddd, 1H), 7.31 (dd, 1H), 9.22 (s, 1H).

### Example 65

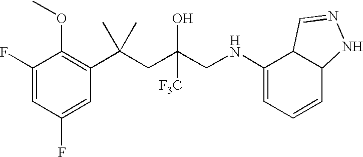

### 4-(3,5-Difluoro-2-methoxyphenyl)-4-methyl-1-(1H-indazol-4-ylamino)-2-(trifluoromethyl)-pentan-2-ol

4-(3,5-Difluoro-2-methoxyphenyl)-2-hydroxy-4-methyl-2-(trifluoromethyl)pentanal

Analogously to the described method of synthesis of 4-(benzo[1,3]dioxol-4-yl)-2-hydroxy-4-methyl-2-trifluoromethyl-pentanal, 90 mg of the title compound is obtained as a colorless oil. ¹H-NMR (CDCl₃), □ (ppm)=1.39 (s, 3H), 1.45 (s, 3H), 2.33 (d, 1H), 3.18 (d, 1H), 3.59 (s, 1H), 3.98 (d, 3H), 6.15 (dm, 1H), 6.72-6.82 (m, 1H), 9.24 (s, 1H)

Analogously to the production of Example 30, the corresponding imine is obtained from 35 mg of 4-amino-1H-indazole and 85 mg of 4-(3,5-difluoro-2-methoxyphenyl)-2-hydroxy-4-methyl-2-(trifluoromethyl)pentanal and further reduced to 43 mg of the title compound with 99 mg of sodium cyanoborohydride. ¹H-NMR (CDCl₃), □ (ppm)=1.45 (s, 1H), 1.59 (s, 3H), 2.26 (d, 1H), 2.71 (d, 1H), 3.22 (d, 1H), 3.34 (d, 1H), 4.01 (d, 3H), 5.81 (d, 1H), 6.78 (ddd, 1H), 6.88 (d, 1H), 6.93 (ddd, 1H), 7.15 (t, 1H), 7.90 (s, 1H)

### Example 66

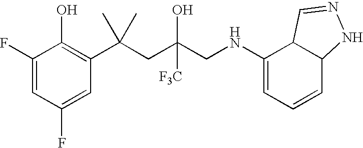

### 4-(3,5-Difluoro-2-hydroxyphenyl)-4-methyl-1-(1H-indazol-4-ylamino)-2-(trifluoromethyl)-pentan-2-ol

Analogously to the production of Example 3, 14 mg of the title compound is obtained from the reaction of 37 mg of 4-(3,5-difluoro-2-methoxyphenyl)-4-methyl-1-(1H-indazol-4-ylamino)-2-(trifluoromethyl)-pentan-2-ol and 1.3 ml of boron tribromide (1 M in dichloromethane). ¹H-NMR (CDCl₃), □ (ppm)=1.50 (s, 3H), 1.62 (s, 3H), 2.30 (d, 1H), 2.81 (d, 1H), 3.27 (d, 1H), 3.39 (d, 1H), 5.73 (d, 1H), 6.78 (dt, 1H), 6.87 (d, 1H), 6.94 (dt, 1H), 7.12 (t, 1H), 7.89 (s, 1H)

### Example 67

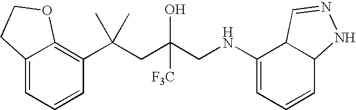

### 4-(2,3-Dihydrobenzofuranyl)-4-methyl-1-(1H-indazol-4-ylamino)-2-(trifluoromethyl)-pentan-2-ol

Analogously to the production of Example 30, the corresponding imine is obtained from 32 mg of 4-amino-1H-indazole and 75 mg of 4-(2,3-dihydrobenzofuranyl)-2-hydroxy-4-methyl-2-(trifluoromethyl)pentanal and further reduced with 134 mg of sodium cyanoborohydride to 64 mg of the title compound. ¹H-NMR (CDCl₃), □ (ppm)=1.45 (s, 3H), 1.57 (s, 3H), 2.31 (d, 1H), 2.64 (d, 1H), 3.05-3.19 (m, 2H), 3.21 (d, 1H), 3.39 (d, 1H), 4.48-4.63 (m, 2H), 5.76 (d, 1H), 6.85 (d, 1H), 6.92 (t, 1H), 7.05-7.23 (m, 3H), 7.85 (s, 1H)

### Example 68

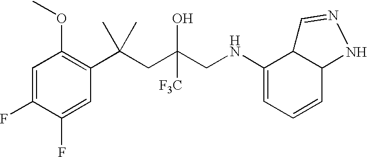

### 4-(4,5-Difluoro-2-methoxyphenyl)-4-methyl-1-(1H-indazol-4-ylamino)-2-(trifluoromethyl)-pentan-2-ol

4-(4,5-Difluoro-2-methoxyphenyl)-2-hydroxy-4-methyl-2-(trifluoromethyl)pentanal

Analogously to the described method of synthesis of 4-(benzo[1,3]dioxol-4-yl)-2-hydroxy-4-methyl-2-trifluoromethyl-pentanal, 695 mg of the title compound is obtained as a colorless oil. ¹H-NMR (CDCl₃), □ (ppm)=1.38 (s, 3H), 1.44 (s, 3H), 2.23 (d, 1H), 3.29 (d, 1H), 3.56 (s, 1H), 3.83 (s, 3H), 6.76 (dd, 1H), 6.96 (dd, 1H), 9.08 (s, 1H)

Analogously to the production of Example 30, the corresponding imine is obtained from 82 mg of 4-amino-1H-indazole and 200 mg of 4-(4,5-difluoro-2-methoxyphenyl)-2-hydroxy-4-methyl-2-(trifluoromethyl)pentanal and further reduced with 319 mg of sodium cyanoborohydride to 155 mg of the title compound. ¹H-NMR (CDCl₃), □ (ppm)=1.44 (s, 3H), 1.58 (s, 3H), 2.24 (d, 1H), 2.71 (d, 1H), 3.19 (d, 1H), 3.32 (bd, 1H), 3.82 (s, 3H), 5.63 (d, 1H), 6.70 (dd, 1H), 6.87 (d, 1H), 7.13 (t, 1H), 7.24 (dd, 1H), 7.87 (s, 1H)

### Example 69

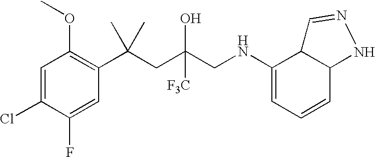

### 4-(4-Chloro-5-fluoro-2-methoxyphenyl)-4-methyl-1-(1H-indazol-4-ylamino)-2-(trifluoromethyl)-pentan-2-ol

4-(4-Chloro-5-fluoro-2-methoxyphenyl)-2-hydroxy-4-methyl-2-(trifluoromethyl)pentanal

Analogously to the described method of synthesis of 4-(benzo[1,3]dioxol-4-yl)-2-hydroxy-4-methyl-2-trifluoromethyl-pentanol, 1.55 g of 4-(4-chloro-5-fluoro-2-methoxyphenyl)-2-hydroxy-4-methyl-2-(trifluoromethyl)pentanal is obtained as fraction 1 and 1.32 g of 4-(4-chloro-5-fluoro-2-methoxyphenyl)-2-hydroxy-4-methyl-2-(trifluoromethyl)pentanol is obtained as fraction 2.

Fraction 1: ¹H-NMR (CDCl₃), □ (ppm)=1.36 (s, 3H), 1.43 (s, 3H), 2.28 (d, 1H), 3.27 (d, 1H), 3.57 (s, 1H), 3.85 (s, 3H), 6.84 (d, 1H), 6.93 (d, 1H), 9.11 (s, 1H)

Fraction 2: ¹H-NMR (CDCl₃), □ (ppm)=1.4 (s, 3H), 1.48 (s, 3H), 2.23 (d, 1H), 2.47 (d, 1H), 2.91 (s, 1H), 3.35 (dd, 1H), 3.5 dd, 1H), 3.83 (s, 3H), 6.87 (d, 1H), 7.1 (d, 1H)

Analogously to the production of Example 30, the corresponding imine is obtained from 78 mg of 4-amino-1H-indazole and 200 mg of 4-(4-chloro-5-fluoro-2-methoxyphenyl)-2-hydroxy-4-methyl-2-(trifluoromethyl)pentanal and further reduced to 116 mg of the title compound with 280 mg of sodium cyanoborohydride. ¹H-NMR (CDCl₃), □ (ppm)=1.43 (s, 3H), 1.58 (s, 3H), 2.23 (d, 1H), 2.71 (d, 1H), 3.19 (d, 1H), 3.31 (bd, 1H), 3.83 (s, 3H), 5.55 (d, 1H), 6.85 (s, 1H), 6.87 (d, 1H), 7.14 (t, 1H), 7.21 (d, 1H), 7.88 (s, 1H)

### Example 70

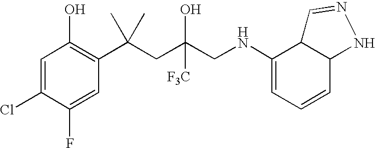

### 4-(4-Chloro-5-fluoro-2-hydroxyphenyl)-4-methyl-1-(1H-indazol-4-ylamino)-2-(trifluoromethyl)-pentan-2-ol

Analogously to the production of Example 3, 30 mg of the title compound is obtained from the reaction of 100 mg of 4-(4-chloro-5-fluoro-2-methoxyphenyl)-4-methyl-1-(1H-indazol-4-ylamino)-2-(trifluoromethyl)-pentan-2-ol and 3.3 ml of boron tribromide (1 M in dichloromethane). Flash point=171-173° C.

### Example 71

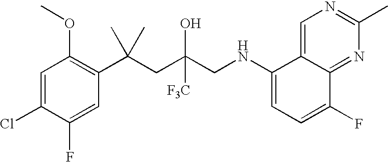

### 4-(4-Chloro-5-fluoro-2-methoxyoxyphenyl)-4-methyl-1-(8-fluoro-2-methylquinazolin-5-ylamino)-2-(trifluoromethyl)-pentan-2-ol

Analogously to Example 25, 4-(4-chloro-5-fluoro-2methoxyphenyl)-2-hydroxy-4-methyl-2-trifluoromethyl-pentanal is reacted with 5-amino-8-fluoro-2-methylquinazoline to form the desired product. ¹H-NMR (CDCl₃); δ=1.45 (s, 3H), 1.56 (s, 3H), 2.30 (d, 1H), 2.73 (d, 1H), 2.90 (s, 3H), 2.99 (s, 1H), 3.14 (dd, 1H), 3.22 (dd, 1H), 3.83 (s, 3H), 4.48 (br., 1H), 5.88 (dd, 1H), 6.79 (d, 1H), 7.17 (d, 1H), 7.31 (dd, 1H), 9.20 (s, 1H).

### Example 72

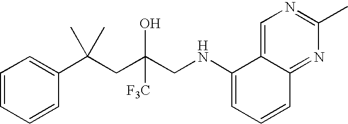

### 4-Methyl-1-(2-methylquinazolin-5-ylamino)-4-phenyl-2-(trifluoromethyl)-pentan-2-ol

2-Hydroxy-4-methyl-4-phenyl-2-trifluoromethyl-pentanal

10.4 g of 4-methyl-2-oxo-4-phenylpentanoic acid (WO98/54159) in 250 ml of dimethylformamide is mixed at −5° C. with 4.1 ml of thionyl chloride and after 15 minutes with 4 ml of methanol. After 15 hours at room temperature, the batch is diluted with water and extracted with ethyl acetate. The organic extracts are washed with water, dried (Na₂SO₄) and concentrated by evaporation, whereby 9.3 g of 4-methyl-2-oxo-4-phenylpentanoic acid-methyl ester is obtained. The latter are mixed in 558 ml of DMF at −5° C. with 15.5 ml (104.63 mmol) of (trifluoromethyl)trimethylsilane and 20.5 g (63.28 mmol) of cesium carbonate and stirred for 16 hours at room temperature. Water is added, extracted with ethyl acetate, the organic phase is washed with water and dried (Na₂SO₄). The intermediate product that is concentrated by evaporation is taken up in 200 ml of THF, and 50 ml of a 1 M solution of tetrabutylammonium fluoride in THF is added. It is stirred for 2 hours, water is added, extracted with ethyl acetate, the organic phase is washed with water and dried (Na₂SO₄). After chromatography on silica gel with hexane-ethyl acetate (0-30%), 8.35 g of 2-hydroxy-4-methyl-4-phenyl-2-(trifluoromethyl)pentanoic acid-methyl ester is obtained. The ester (8.3 g, 28.59 mmol) is dissolved in 180 ml of THF, and over a period of 2.5 hours, 1.52 g (36.20 mmol) of lithium aluminum hydride is added in small portions. After conversion is completed, 5 ml of ethyl acetate is added in drops, and after another 10 minutes, 10 ml of water is carefully added. The precipitate that is formed is filtered out and washed carefully with ethyl acetate. After chromatography on silica gel with hexane-ethyl acetate (0-35%), 5.40 g of 4-methyl-4-phenyl-2-(trifluoromethyl)pentane-1,2-diol is obtained. 5.7 ml (40.3 mmol) of triethylamine and, in portions over 20 minutes, 5 g of pyridine SO₃ complex are added to 2.5 g (9.53 mmol) of diol in 75 ml of dichloromethane and 28 ml of DMSO. It is stirred over 2 hours, and 40 ml of saturated ammonium chloride solution is added. The mixture is stirred for another 15 minutes, the phases are separated, and it is extracted with dichloromethane. It is washed with water and dried on sodium sulfate. The solvent is removed in a vacuum, and 3 g of product is obtained. ¹H-NMR (CDCl₃): δ=1.34 (s, 3H), 1.44 (s, 3H), 2.34 (d, 2H), 2.66 (d, 1H), 3.64 (s, 1H), 7.03-7.41 (m, 4H), 8.90 (s, 1H).

Analogously to Example 25, 2-hydroxy-4-methyl-4-phenyl-2-trifluoromethyl-pentanal is reacted with 5-amino-2-methylquinazoline to form the desired product.

¹H-NMR (CDCl₃); δ=1.46 (s, 3H), 1.59 (s, 3H), 2.30 (d, 1H), 2.37 (d, 1H), 2,84 (s, 3H), 3.10 (dd, 1H), 3.27 (dd, 1H), 4.70 (br., 1H), 6.12 (d, 1H), 7.26 (d, 1H), 7.28 (t, 1H), 7.39 (t, 2H), 7.48 (d, 2H), 7.58 (t, 1H), 9.22 (s, 1H).

### Example 73

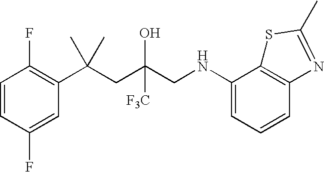

### 4-(2,5-Difluorophenyl)-4-methyl-1-(2-methyl-benzothiazol-7-ylamino)-2-(trifluoromethyl)-pentan-2-ol

110 mg (0.66 mmol) of 2-methyl-benzothiazol-7-ylamine in 1 ml of acetic acid is mixed with 150 mg (0.53 mmol) of 4-(2,5-difluoro-phenyl)-2-hydroxy-4-methyl-2-trifluoromethyl-pentanal, dissolved in 10 ml of dichloroethane, refluxed for 4 hours on a molecular sieve (4 A) and stirred for another 16 hours at room temperature. The mixture was dispersed between water and dichloromethane, extracted with dichloromethane, and the combined organic phases were washed (saturated NaCl solution), dried (Na₂SO₄) and concentrated by evaporation. The intermediate product (97 mg) obtained after chromatography on silica gel with hexane/ethyl acetate (20%) is taken up in acetic acid, mixed with 10 mg of NaBH₄, and the mixture is stirred for 4 hours at room temperature. It is dispersed between water and dichloromethane, extracted, the combined organic phases (saturated NaCl solution) are washed, dried (Na₂SO₄) and concentrated by evaporation. 90 mg of product, which can be crystallized from hexane/diethyl ether, is obtained. MS (CI): 445 (M+H); ¹H-NMR (CDCl₃): δ=1.61 (s, br, 6H), 2.26 (d, 1H), 2.50 (d, 1H), 2.83 (s, 3H), 3.15 (s, 1H), 3.27 (d, br, 2H), 3.49 (m, 1H), 6.02 (d, 1H), 6.82-7.02 (m, 2H), 7.10-7.25 (m, 2H), 7.45 (dd, 1H).

### Example 74

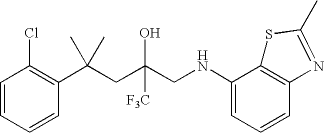

### 4-(2-Chlorophenyl)-4-methyl-1-(2-methyl-benzothiazol-7-ylamino)-2-(trifluoromethyl)-pentan-2-ol

Analogously to Example 5, the desired product is obtained from 7-amino-2-methylbenzothiazole (Libeer et al. Bull. Soc. Chim. Belg.; 1971; 80; 43-47) and 4-(2-chlorophenyl)-2-hydroxy-4-methyl-2-trifluoromethyl-pentanal. ¹H-NMR (300 MHz, CDCl₃): □=1.60 (3H), 1.73 (3H), 2.20 (1H), 2.80 (3H), 3.09 (1H), 3.18 (1H), 3.23 (4H), 5.78 (1H), 7.13 (1H), 7.20-7.35 (2H), 7.37-7.45 (2H), 7.60 (1H).

### Example 75

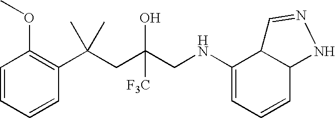

### 4-(2-Methoxyphenyl)-4-methyl-1-(1H-indazol-4-ylamino)-2-(trifluoromethyl)-pentan-2-ol

4-(2-Methoxyphenyl)-2-hydroxy-4-methyl-2-(trifluoromethyl)pentanal

Analogously to the described method of synthesis of 4-(benzo[1,3]dioxol-4-yl)-2-hydroxy-4-methyl-2-trifluoromethyl-pentanal (Example 32), 7.9 mg of the title compound is obtained as a colorless oil starting from 28.6 g of 4-(2-methoxyphenyl)-4-methyl-2-oxo-pentanoic acid ethyl ester (WO 98/54159). ¹H-NMR (CDCl₃), □ (ppm)=1.40 (s, 3H), 1.47 (s, 3H), 2.2 (d, 1H), 3.46 (d, 1H), 3.60 (s, 1H), 3.88 (s, 3H), 6.83-6.94 (m, 2H), 7.13 (dd, 1H), 7.24 (dt, 1H), 8.94 (s, 1H)

Analogously to the production of Example 30, the corresponding imine is obtained from 139 mg of 4-amino-1H-indazole and 300 mg of 4-(2-methoxyphenyl)-2-hydroxy-4-methyl-2-(trifluoromethyl)pentanal and further reduced with 627 mg of sodium cyanoborohydride to 310 mg of the title compound.

¹H-NMR (CDCl₃), □ (ppm)=1.48 (s, 3H), 1.60 (s, 3H), 2.33 (d, 1H), 2.77 (d, 1H), 3.17 (dd, 1H), 3.36 (dd, 1H), 3.88 (s, 3H), 3.98-4.08 (m, 1H), 5.66 (d, 1H), 6.83 (d, 1H), 6.94 (dd, 1H), 7.04 (dt, 1H), 7.09 (t, 1H), 7.32 (dt, 1H), 7.43 (dd, 1H), 7.86 (s, 1H)

### Example 76

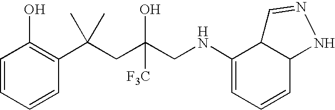

### 4-(2-Hydroxyphenyl)-4-methyl-1-(1H-indazol-4-ylamino)-2-(trifluoromethyl)-pentan-2-ol

Analogously to the production of Example 3, 15 mg of the title compound is obtained from the reaction of 20 mg of 4-(2-methoxyphenyl)-4-methyl-1-(1H-indazol-4-ylamino)-2-(trifluoromethyl)-pentan-2-ol and 0.75 ml of boron tribromide (1 M in dichloromethane).

¹H-NMR (CDCl₃), □ (ppm)=1.52 (s, 3H), 1.62 (s, 3H), 2.32 (d, 1H), 2.90 (d, 1H), 3.23 (d, 1H), 3.43 (dd, 1H), 4.22 (bs, 1H), 5.75 (d, 1H), 6.72 (d, 1H), 6.79 (d, 1H), 6.97 (t, 1H), 7.04-7.16 (m, 2H), 7.39 (d, 1H), 7.93 (s, 1H)

### Examples 77 and 78

### (−)-4-(2-Hydroxyphenyl)-4-methyl-1-(1H-indazol-4-ylamino)-2-(trifluoromethyl)-pentan-2-ol and (+)-4-(2-Hydroxyphenyl)-4-methyl-1-(1H-indazol-4-ylamino)-2-(trifluoromethyl)-pentan-2-ol

Separation of (+/−)-4-(2-Hydroxyphenyl)-4-methyl-1-(1H-indazol-4-ylamino)-2-(trifluoromethyl)-pentan-2-ol

The enantiomer mixture is separated by chromatography on chiral carrier material (CHIRALPAK AD®, DAICEL Company) with hexane/ethanol (70:30, vvv). Thus obtained are

Enantiomer A: MS (ei) M⁺=392 and

Enantiomer B: MS (ei) M⁺=392

### Example 79

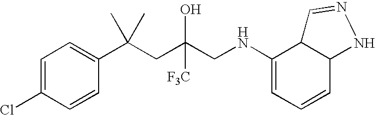

### 4-(4-Chlorophenyl)-4-methyl-1-(1H-indazol-4-ylamino)-2-(trifluoromethyl)-pentan-2-ol

2-(4-Chlorophenyl)-2-methylpropanal

10 g of 4-chlorobenzyl cyanide and 14.3 ml of methyl iodide in 140 ml DMF are mixed at 0° C. in portions with sodium hydride (60% in oil). It is stirred overnight and then mixed with water and ethyl acetate. The phases are separated, and the aqueous phase is extracted with ethyl acetate.

It is thoroughly extracted with water, washed with brine, dried with sodium sulfate and concentrated by evaporation in a vacuum. After chromatography on silica gel (hexane/ethyl acetate 95:5), 11.73 g of 2-(4-chlorophenyl)-2-methylpropionitrile is obtained as a colorless oil. The latter is slowly mixed in toluene at −78° C. with 55.4 ml of diisobutylaluminum hydride solution (20% in toluene), and after 4 hours at −78° C., 50 ml of ethyl acetate was added in drops. It is stirred overnight while being heated to room temperature, and water is added. After filtration through diatomaceous earth, the phases are separated, and the aqueous phase is extracted with ethyl acetate. It is washed with water and brine, dried with sodium sulfate and concentrated by evaporation in a vacuum. After chromatography on silica gel (hexane/ethyl acetate 95:5), 10.2 g of 2-(4-chlorophenyl)-2-methylpropanal is obtained as a colorless oil. ¹H-NMR (CDCl₃), □ (ppm)=1.46 (s, 6H), 7.20 (d, 1H), 7.29-7.43 (m, 3H), 9.48 (s, 1H)

4-(4-Chlorophenyl)-4-methyl-2-oxo-valeric acid

A solution of 15.04 g of 2-diethylphosphono-2-ethoxyacetic acid-ethyl ester in 50 ml of tetrahydrofuran is mixed with 30 ml of a 2 M solution of lithium diisopropylamide in tetrahydrofuran-heptane-toluene while being cooled with ice within 20 minutes and stirred for 15 minutes at 0° C. Within 30 minutes, a solution of 10.2 g of 2-(4-chlorophenyl)-2-methylpropanal in 50 ml of tetrahydrofuran at 0° C. is added thereto. After 20 hours at room temperature, 2N sulfuric acid is added, extracted with ethyl acetate, dried (Na₂SO₄) and concentrated by evaporation. The crude product is saponified with 200 ml of 2 M sodium hydroxide solution/400 ml of ethanol. 13.8 g of acid, which is refluxed for 3 hours with 300 ml of 2N sulfuric acid and 100 ml of glacial acetic acid while being stirred vigorously, is obtained. After extraction with ethyl acetate and washing with water, 10.9 g of 4-(4-chlorophenyl)-4-methyl-2-oxo-valeric acid is obtained as a red oil.

¹H-NMR (CDCl₃), □ (ppm)=1.47 (s, 6H), 3.28 (s, 2H), 7.28 (m, 4H), 7.73 (bs, 1H)

4-(4-Chlorophenyl)-2-hydroxy-4-methyl-2-(trifluoromethyl)-pentan-1-al

Analogously to the synthesis of 4-(3-chloro-2-methoxy-phenyl)-2-hydroxy-4-methyl-2-trifluoromethyl-pentanal (Example 45), 4.22 g of 4-(4-chlorophenyl)-2-hydroxy-4-methyl-2-(trifluoromethyl)pentan-1-ol is obtained as a colorless oil by esterification of 10.9 g of 4-(4-chlorophenyl)-4-methyl-2-oxo-valeric acid in ethanol/sulfuric acid, reaction of the product with (trifluoromethyl)trimethylsilane and tetrabutylammonium fluoride and reduction of the formed hydroxy ester with lithium aluminum hydride.

¹H-NMR (CDCl₃), □ (ppm)=1.39 (s, 3H), 1.49 (s, 3H), 2.07 (d, 1H), 2.19 (d, 1H), 2.83 (bs, 1H), 3.27 (d, 1H), 3.41 (d, 1H), 7.26-7.38 (m, 4H).

6.8 ml (33.3 mmol) of triethylamine and, in portions over 20 minutes, 1.5 g of pyridine SO₃ complex are added to 2 g (6.7 mmol) of diol in 50 ml of dichloromethane and 22 ml of DMSO. It is stirred over 5 hours, and 40 ml of saturated ammonium chloride solution is added. The mixture is stirred for another 15 minutes, the phases are separated, and it is extracted with dichloromethane. It is washed with water and dried on sodium sulfate. The solvent is removed in a vacuum, and after chromatography on silica gel (hexane/ethyl acetate 0-30%), 1.27 g of product is obtained. ¹H-NMR (CDCl₃): δ=1.34 (s, 3H), 1.44 (s, 3H), 2.34 (d, 2H), 2.66 (d, 1H), 3.64 (s, 1H), 7.23-7.31 (m, 4H), 8.90 (s, 1H). Analogously to the production of Example 30, the corresponding imine is obtained from 158 mg of 4-amino-1H-indazole and 350 mg of 4-(4-chlorophenyl)-2-hydroxy-4-methyl-2-(trifluoromethyl)pentanal, and 100 mg of the imine was further reduced with 216 mg of sodium cyanoborohydride to 68 mg of the title compound. ¹H-NMR (CDCl₃), □ (ppm)=1.42 (s, 3H), 1.59 (s, 3H), 2.19 (d, 1H), 2.31 (d, 1H), 3.11 (d, 1H), 3.22 (d, 1H), 5.67 (d, 1H), 6.90 (d, 1H), 7.18 (t, 1H), 7.35 (d, 2H), 7.42 (d, 2H), 7.89 (s, 1H)

### Example 80

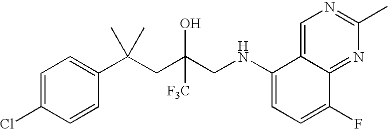

### 4-(4-Chlorophenyl)-4-methyl-1-(8-fluoro-2-methylquinazolin-5-ylamino)-2-(trifluoromethyl)-pentan-2-ol

Analogously to Example 25, 4-(4-chlorophenyl)-2-hydroxy-4-methyl-2-trifluoromethyl-pentanal is reacted with 5-amino-8-fluoro-2-methylquinazoline to form the desired product.

¹H-NMR (CDCl₃); δ=1.45 (s, 3H), 1.58 (s, 3H), 2.29 (d, 1H), 2.36 (d, 1H), 2.79 (br., 1H), 2.90 (s, 3H), 3.05 (dd, 1H), 3.20 (dd, 1H), 4.37 (br., 1H), 5.98 (dd, 1H), 7.29 (dd, 1H), 7.38 (t, 2H), 7.49 (d, 2H), 9.21 (s, 1H).

### Example 81

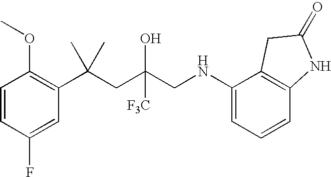

### 4-{[4-(5-Fluoro-2-methoxyphenyl)-2-hydroxy-4-methyl-2-(trifluoromethyl)-pentyl]amino}-1,3-dihydroindol-2-one

Dimethyl-2-(2,6-dinitrophenyl)-malonate

42.95 g (311.03 mmol) of dimethylmalonate is dissolved in 300 ml of N,N-dimethylformamide and mixed in portions with 35.15 g (296.22 mmol) of potassium-tert. butylate. After the tert-butanol that was produced was distilled off, the reaction mixture is cooled to 20° C. 30 g (148.11 mmol) of 2,6-dichlorbenzene is quickly added in portions to the mixture. After three hours of stirring at 90° C., it is stirred overnight at room temperature. The reaction mixture is added to 800 ml of 1% NaOH solution (cooled with ice) and extracted three times with methyl-tert. butyl ether. The combined ether phases are discarded after TLC monitoring. The aqueous phase is carefully acidified while being cooled in an ice bath with concentrated nitric acid (w=65%). After being extracted six times with methyl-tert-butyl ether, conventional working-up of the combined organic extracts (water, brine, drying, filtering and spinning-off of the solvent) yields a residue that is chromatographed on silica gel (mobile solvent ethyl acetate/hexane). 12.09 g (27.09%) of the desired compound is isolated.

Methyl-(2,6-Dinitrophenyl)-acetate

10.08 g (33.8 mmol) of dimethyl-2-(2,6-dinitrophenyl)-malonate is mixed in 54 ml of glacial acetic acid with 2.7 ml of perchloric acid and refluxed at 125° C. In this case, the ethyl acetate that is produced was distilled off. After 90 minutes, the reaction is brought to a halt since starting material is no longer present according to TLC monitoring. The reaction mixture is poured into ice water and extracted three times with ethyl acetate. The combined organic extracts are shaken with 5% sodium bicarbonate solution, with water and with brine. After the organic phase is dried, the desiccant is filtered off and the solvent is spun off, a residue that is chromatographed on silica gel (mobile solvent ethyl acetate/hexane) remains. 4.69 g of the (2,6-dinitrophenyl)-acetic acid is isolated which then is esterified with methanol (16 ml) and concentrated sulfuric acid (0.4 ml). To this end, the acid and the reagents are refluxed for seven hours. The methanol is spun off, and the residue is worked up in the usual way. After chromatography on silica gel (mobile solvent ethyl acetate/hexane), 4.43 g (89%) of the desired ester is obtained.

4-Amino-1,3-dihydroindol-2-one

4.43 g (18.45 mmol) of methyl-(2,6-dinitrophenyl)-acetate is added to 38.8 ml of glacial acetic acid and 11 ml of water and mixed with 3.75 g of iron powder and stirred for four more hours. In this case, heating to 40 to 60° C. takes place. The reaction mixture is added to ice water, mixed with ethyl acetate and stirred vigorously for ten minutes. The mixture is filtered with a glass-fiber filter, the organic phase is separated, and the aqueous phase is extracted twice more with ethyl acetate. The combined organic extracts are washed with brine, dried, and the solvent is spun off after the desiccant is filtered off. The residue is chromatographed on silica gel (mobile solvent methanol/dichloromethane). 2.38 g of the 4-nitro-indol-2-one is isolated. The nitro compound, however, is mixed in glacial acetic acid/water with 2.7 g of iron powder and passes through the above-described cycle another time. 1.63 g of the desired amine is now isolated.

Analogously to Example 27, 4-(5-fluoro-2-methoxyphenyl)-2-hydroxy-2-trifluoromethyl-pentanal is reacted with 4-amino-1,3-dihydroindol-1-one to form the desired product.

¹H-NMR (CDCl₃): δ=1.44 (s, 3H), 1.58 (s, 3H), 2.23 (d, 1H), 2.70 (d, 1H), 3.04 (s, 1H), 3.09 (d, 1H), 3.19 (s, 2H), 3.21 (d, 1H), 3.85 (s, 3H), 5.71 (d, 1H), 6.32 (d, 1H), 6.84 (dd, 1H), 6.92-7.01 (m, 2H), 7.14 (dd, 1H), 7.78 (s, br, 1H)

### Example 82

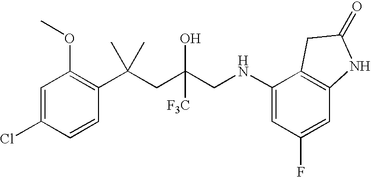

### 4-{[4-(5-Chloro-2-methoxyphenyl)-2-hydroxy-4-methyl-2-(trifluoromethyl)pentyl]amino}-1,3-dihydro-6-fluoroindol-2-one

180 mg (0.60 mmol) of 4-(4-chloro-2-methoxyphenyl)-2-hydroxy-4-methyl-2-trifluoromethyl-pentanal and 100 mg (0.60 mmol) of 4-amino-6-fluoroindol-2-one are introduced into 15 ml of dichloroethane and 3 ml of acetic acid, about 100 mg of powdered molecular sieve (4 A) is added, and the mixture is refluxed for 6 hours. After cooling, it is filtered off, the filtrate is dispersed between dichloromethane and saturated NaHCO₃ solution, and the phases are separated. The aqueous phase is extracted several times with dichloromethane, the combined organic phases are washed with water and saturated NaCl solution, and dried with Na₂SO₄. The solvent is removed, and the crude product is chromatographed on silica gel (eluant hexane/ethyl acetate 20-100%). 40 mg of imine, which is taken up in 5 ml of methanol, is obtained, mixed with a few drops of acetic acid and about 20 mg of sodium cyanoborohydride and stirred for 2 hours at room temperature. The mixing is brought to a halt by adding saturated NaHCO₃ solution. It is extracted with dichloromethane, washed with saturated NaCl solution and dried with Na₂SO₄. Chromatographic purification of the crude product (silica gel, eluant hexane/ethyl acetate 20-100%) yields 4 mg of the desired product. MS (ESI): 475/477 (M+H); ¹H-NMR (CDCl₃): δ=1.46 (s, 3H), 1.57 (s, 3H), 2.29 (d, 1H), 2.60 (d, 1H), 2.78 (s, 1H), 2.99-3.21 (m, 4H), 3.47 (t, br, 1H), 3.88 (s, 3H), 5.52 (dd, 1H), 6.08 (dd, 1H), 6.89 (d, 1H), 7.00 (dd, 1H), 7.32 (dd, 1H), 7.68 (s, br, 1H).

### Examples 83-92

General Operating Instructions for the Production of N-Methyl-benzimidazole Derivatives:

0.70 mmol of the substituted pentanal and 1.05 mmol of the amine component (4-methyl-1H-benzimidazol-4-ylamine, cf., e.g., V. Milata, D. Ilavsky, J. Saloò, Bull. Soc. Chim. Belg. 1997, 106, 731-732; 3-methyl-3H-benzimidazol-4-ylamine, cf., e.g., M. Kamel, M. I. Ali, M. M. Kamel, Tetrahedron 1966, 22, 3351-3354 ) are introduced with about 80 mg of molecular sieve (4 Å, powdered) into 10 ml of dichloroethane and 1 ml of acetic acid and refluxed for about 7 hours. After the cooling, the mixture is mixed with phosphate buffer solution (1 M, pH 7) and dichloromethane, filtered off, and the phases are separated. The aqueous phase is extracted several times with dichloromethane, the combined organic phases are washed with water and saturated NaCl solution and dried with Na₂SO₄. The solvents are removed in a rotary evaporator, and the intermediate product is purified by chromatography on silica gel (eluant hexane/ethyl acetate). The imine that is formed is further taken up in a little methanol, some drops of acetic acid are added, and NaCNBH₃ (2-3 equivalents) is added. The solution is stirred for 3 hours at room temperature, the reaction is completed by adding phosphate buffer solution (1 M, pH 7) and extracted with dichloromethane. The combined organic phases are washed with water and saturated NaCl solution and dried with Na₂SO₄. The crude product is filtered on silica gel with hexane/ethyl acetate.

| Example Name | Structure | Yield | MS | ¹H-NMR (300 MHz) |
|---|---|---|---|---|
| 1,1,1-Trifluoro-4- (4-chloro-2- methoxy-phenyl)- 4-methyl-2-(4- methyl-1H- benzimidazol-4- ylamine) | 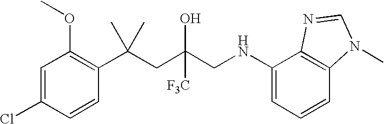 | 38% | 456/458 (M + H, CI) | 1.36 (s, 3H), 1.53 (s, 3H), 2.01 (d, 1H), 2.86 (d, 1H), 2.84-2.93 (m, 1H), 3.02-3.10 (m, 1H), 3.75 (s, 3H), 3.84 (s, 3H), 5.01 (t, br, 1H), 5.76 (d, 1H), 6.22 (s, 1H), 6.74 (d, 1H), 6.85-6.99 (m, |
| # 3H), 7.25 (d, 1H), 7.99 (s, 1H). (d₆-DMSO) | | | | |
| 1,1,1-Trifluoro-4- (4-chloro-2- methoxy-phenyl)- 4-methyl-2-(3- methyl-3H- benzimidazol-4- ylamine) | 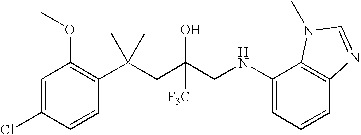 | 38% | — | 1.42 (s, 3H), 1.64 (s, 3H), 2.00 (d, 1H), 2.77 (d, 1H), 3.00 (d, 1H), 3.13 (d, 1H), 3.81 (s, 3H), 4.11 (s, 3H), 5.70 (d, 1H), 6.84-7.07 (m, 4H), 7.33 (d, 1H), 8.04 (s, 1H). (CD₃OD) |
| 1,1,1-Trifluoro-4- (5-fluoro-2- methoxy-phenyl)- 4-methyl-2-(4- methyl-1H- benzimidazol-4- ylamine) | 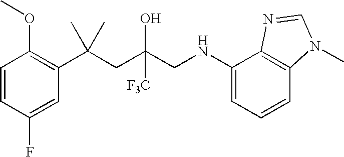 | 28% | 440 (M + H, ESI) | 1.43 (s, 3H), 1.62 (s, 3H), 2.08 (d, 1H), 3.01 (d, 1H), 3.07 (d, 1H), 3.19 (d, 1H), 3.82 (s, 3H), 3.87 (s, 3H), 5.79 (d, 1H), 6.73-6.88 (m, 3H), 7.03-7.13 (m, 2H), 8.11 (s, 1H). (CD₃OD) |
| 1,1,1-Trifluoro-4- (5-fluoro-2- methoxy-phenyl)- 4-methyl-2-(3- methyl-3H- benzimidazol-4- ylamine) | 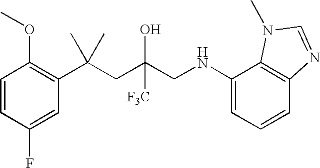 | 18% | — | 1.56 (s, 3H), 1.70 (s, 3H), 2.28 (d, 1H), 3.01 (d, 1H), 3.06 (d, 1H), 3.25 (d, 1H), 3.94 (s, 3H), 4.14 (s, 3H), 5.94 (d, 1H), 6.88 (dd, 1H), 6.97-7.05 (m, 1H), 7.12 (dd, 1H), 7.22 (dd, 1H), 7.28 (d, 1H), 7.82 (s, |
| # 1H). (CDCl₃) | | | | |
| 1,1,1-Trifluoro-4- (2,5-difluoro- phenyl)-4-methyl- 2-(4-methyl-1H- benzimidazol-4- ylamine) | 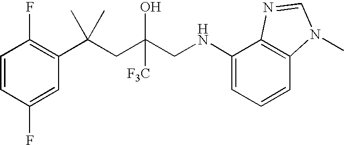 | 40% | 427 (M, EI) | 1.42 (s, 3H), 1.50 (s, 3H), 2.21 (d, 1H), 2.45 (d, 1H), 3.21 (m, 2H), 3.76 (s, 3H), 5.25 (t, br, 1H), 5.98 (d, 1H), 6.55 (s, 1H), 6.76 (d, 1H), 6.93-7.16 (m, 4H), 7.98 (s, 1H). (d₆-DMSO) |
| 1,1,1-Trifluoro-4- (2,5-difluoro- phenyl)-4-methyl-2-(3-methyl-3H- benzimidazol-4- ylamine) | 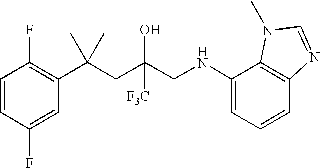 | 90% | 428 (M + H, ESI) | 1.47 (s, 3H), 1.60 (s, 3H), 2.25 (d, 1H), 2.57 (d, 1H), 3.09 (d, 1H), 3.19 (d, 1H), 4.02 (s, 3H), 5.94 (d, 1H), 6.80-6.93 (m, 2H), 6.99-7.13 (m, 2H), 7.25 (d, 1H), 7.88 (s, 1H). (CDCl₃) |

General Operating Instructions for Methyl Ether Cleavage in N-Methylbenzimidazole Derivatives:

About 0.1 mmol of the methyl ether is introduced into about 3 ml of dichloromethane, and about 1 ml of a BBr₃ solution (1 M in dichloromethane) is added. It is stirred for 2 hours at room temperature, diluted with ethyl acetate, the solution is added to saturated NaHCO₃ solution, the phases are separated and extracted with ethyl acetate. The combined organic phases are washed with water and saturated NaCl solution and dried with Na₂SO₄. The crude product is crystallized from diethyl ether/hexane. Yields of about 50% are obtained.

| Name | Structure | MS | ¹H-NMR (300 MHz, CDCl₃) |
|---|---|---|---|
| 1,1,1-Trifluoro-4- (4-chloro-2- hydroxy-phenyl)-4- methyl-2-(4- methyl-1H- benzimidazol-4- ylamine) | 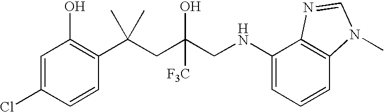 | 442/444 (M + H, ESI) | 1.32 (s, 3H), 1.64 (s, 3H), 2.02 (m, 2H), 3.00-3.16 (m, 2H), 4.04 (s, 3H), 5.98 (d, 1H), 6.68-6.74 (m, 1H), 7.04 (d, 1H), 7.23-7.33 (m, 3H), 8.98 (s, 1H). (CD₃OD) |
| 1,1,1-Trifluoro-4- (4-chloro-2- hydroxy-phenyl)-4- methyl-2-(3- methyl-3H- benzimidazol-4- ylamine) | 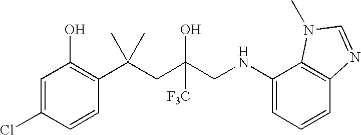 | 442/444 (M + H, ESI) | |
| 1,1,1-Trifluoro-4- (5-fluoro-2- hydroxy-phenyl)-4- methyl-2-(4- methyl-1H- benzimidazol-4- ylamine) | 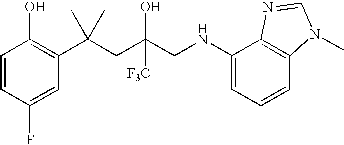 | 426 (M + H, ESI) | 1.47 (s, 3H), 1.64 (s, 3H), 1.97-2.03 (m, 2H), 3.06 (d, 1H), 3.27 (d, 1H), 3.85 (s, 3H), 5.86 (d, 1H), 6.57-6.82 (m, 3H), 6.98-7.06 (m, 2H), 8.01 (s, 1H). (CD₃OD) |
| 1,1,1-Trifluoro-4- (5-fluoro-2- hydroxy-phenyl)-4- methyl-2-(3- methyl-3H- benzimidazol-4- ylamine) | 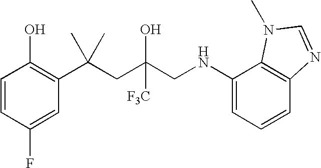 | 426 (M + H, ESI) | 1.47 (s, 3H), 1.63 (s, 3H), 1.92-2.04 (m, 1H), 2.87 (d, 1H), 3.09-3.18 (m, 1H), 4.15 (s, 3H), 5.84 (d, 1H), 6.40 (dd, 1H), 6.50-6.57 (m, 1H), 6.92-7.06 (m, 3H), 8.09 (s, 1H). (CD₃OD) |

### Example 93

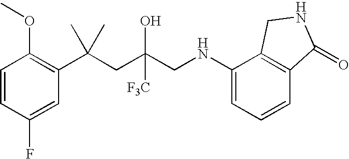

### 4-{[4-(5-Fluoro-2-methoxyphenyl)-2-hydroxy-4-methyl-2-(trifluoromethyl)pentyl]amino}-2,3-dihydroisoindolon-1(2H)-one

2-Methyl-3-nitrobenzoic acid methyl ester

30 g (165.6 mmol) of 2-methyl-3-nitrobenzoic acid is added to 150 ml of methanol, and it is refluxed for two days after 2.9 ml of concentrated sulfuric acid is added. After cooling, the crystallizate (22.55 g=79%) is suctioned off and used in the next stage.

2-(Bromomethyl)-3-nitrobenzoic acid methyl ester

25.55 g (130.9 mmol) of 2-methyl-3-nitrobenzoic acid methyl ester is added to 300 ml of carbon tetrachloride, and mixed with 25.6 g (141.7 mmol) of N-bromosuccinimide and 62.8 mg of benzoyl peroxide. After seven days of refluxing, the succinimide is suctioned off after cooling, and then the filtrate is spun in to the dry state. The desired compound that is incorporated in crude form into the next stage remains.

2-(Azidomethyl)-3-nitrobenzoic acid methyl ester

10 g (36.5 mmol) of 2-(bromomethyl)-3-nitrobenzoic acid methyl ester is mixed with 36 ml of N,N-dimethylformamide and 24 ml of water. After 3.54 g of sodium azide is added, the batch is stirred overnight. The reaction mixture is diluted with methyl-tert butyl ether, and washed twice with water and once with brine. After drying on sodium sulfate, it is filtered, and the solvent is spun off. The desired azide is obtained in a yield of 89.6% (7.72 g) and further used in crude form.

4-Amino-2,3-dihydroisoindol-1-one

1 g (4.2 mmol) of 2-(azidomethyl)-3-nitrobenzoic acid methyl ester is added to 10 ml of ethanol and 2 ml of glacial acetic acid and mixed with 148.5 mg of Pd/C. After stirring overnight at room temperature under a hydrogen atmosphere, the catalyst is suctioned off via a glass-fiber filter, and the filtrate is concentrated by evaporation until a dry state is reached. The residue is chromatographed on silica gel (ethyl acetate). 391.5 mg (62.4%) of the desired compound is isolated.

Analogously to Example 30, 4-(5-fluoro-2-methoxyphenyl)-2-hydroxy-2-trifluoromethyl-pentanal is reacted with 4-amino-2,3-dihydroisoindol-1-one to form the desired product.

¹H-NMR (DMSO_{D6}): δ=1.37 (s, 3H), 1.54 (s, 3H), 2.03 (d, 1H), 2.81 (d, 1H), 2.81 (d, 1H), 2.90 (dd, 1H), 3.02 (dd, 1H), 3.33 (s, 1H), 3.81 (s, 3H), 4.01-4.14 (m, 2H), 4.77 (br., 1H), 5.76 (s, 1H), 6.17 (d, 1H), 6.88 (d, 1H), 7.01-7.16 (m , 4H), 8.35 (s, 1H)

### Example 94

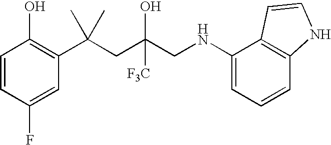

### 4-(5-Fluoro-2-hydroxyoxyphenyl)-1-(1H-indol-4-ylamino)-4-methyl-2-(trifluoromethyl)pentan-2-ol

Analogously to Example 3, 4-(5-fluoro-2-methoxyphenyl)-4-methyl-1-(1H-indol-5-ylamino)-2-(trifluoromethyl)-pentan-2-ol is reacted to form the desired product.

¹H-NMR (CDCl₃); δ=1.49 (s, 3H), 1.61 (s, 3H), 2.31 (d, 1H), 2.75 (d, 1H), 3.26 (d, 1H), 3.43 (d, 1H), 3.60 (s, 1H), 5.67 (d, 1H), 6.33 (m, 1H), 6.63 (dd, 1H), 6.86-6.97 (m, 3H), 7.10 (dd, 1H), 7.15 (dd, 1H), 8.14 (br, 1H)

### Example 95

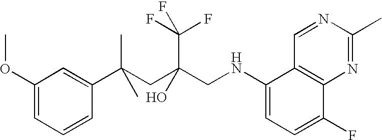

### 1,1,1-Trifluoro-2-[(8-fluoro-2-methyl-quinazolin-5-ylamino)-methyl]-4-(3-methoxyl-phenyl)-4-methyl-pentan-2-ol

Starting from the corresponding precursors, the compound was synthesized analogously to what is described in Example 13. 26.9 mg (17.8%) of the desired compound was isolated in the last step.

¹H-NMR (300 MHz, CD₃OD): □=1.49 (3H), 1.69 (3H), 2.19 (1H), 2.56 (1H), 2.85 (3H), 2.93 (1H), 3.12 (1H), 3.64 (3H), 5.90 (1H), 6.58 (1H), 7.00 (1H), 7.03-7.20 (2H), 7.38 (1H), 9.38 (1H).

### Example 96

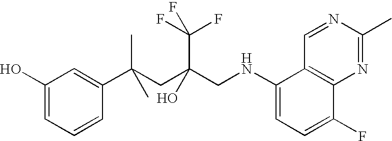

### 3-{4,4,4-Trifluoro-3-[(8-fluoro-2-methyl-quinazolin-5-ylamino)-methyl-3-hydroxy-1,1,-dimethyl-butyl}-phenol

Starting from ether, described in Example 95, the compound was obtained by ether cleavage with BBr₃. 6 mg (29.5%) of the desired compound was isolated in the last step.

¹H-NMR (300 MHz, CD₃OD): □=1.49 (3H), 1.65 (3H), 2.15 (1H), 2.50 (1H), 2.82 (3H), 2.95 (1H), 3.17 (1H), 6.02 (1H), 6.53 (1H), 6.90-7.13 (3H), 7.39 (1H), 9.38 (1H).

### Example 97

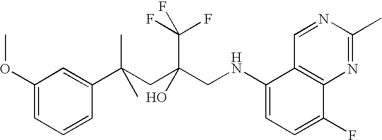

### (+)-1,1,1-Trifluoro-2-[(8-fluoro-2-methyl-cinazolin-5-ylamino)-methyl]-4-(3-methoxy -phenyl)-4-methyl-pentan-2-ol

The compound that is produced according to Example 95 is separated into enantiomers thereof on a chiral column (Chiralpak AD 20□, mobile solvent hexane/isopropanol).

(+)-Enantiomers: Example 97;

(−)-Enantiomers: Example 98.

### Example 98

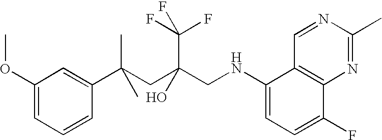

### (−)-1,1,1-Trifluoro-2-[(8-fluoro-2-methyl-cinazolin-5-ylamino)-methyl]-4-(3-methoxy -phenyl)-4-methyl-pentan-2-ol

For conditions for the racemate cleavage, see Example 97.

### Example 99

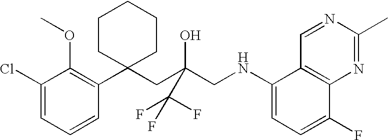

### 3-[1-(3-Chloro-2-methoxy-phenyl-cyclohexyl]-1,1,1-trifluoro-2[(8-fluoro-2-methyl-quinazolin-5-ylamino)-methyl]-propan-2-ol

The compound was synthesized starting from the corresponding precursors and analogously to what is described in the examples above. 32 mg (31.9%) of the desired compound was isolated in the last step.

¹H-NMR (CDCl₃): δ=1.00-2.50 (12H), 2.60-3.30 (6H), 3.79 (3H), 4.15-4.55 (1H), 5.60-5.85 (1H), 6.82 (1H), 6.95 (1H), 7.10-7.50 (2H), 9.10 (1H).

### Example 100

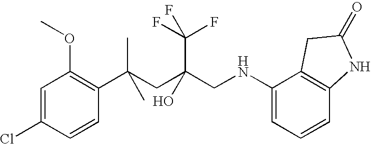

### (−)-4-[4-(4-Chloro-2-methoxy-phenyl)-2-hydroxy-4-methyl-2-trifluoromethyl-pentylamino]-1,3-dihydro-indol-2-one

The compound was synthesized starting from the corresponding precursors and analogously to what is described in the examples above. 117.1 mg (97.7%) of the desired compound was isolated as a racemate in the last step.

¹H-NMR (300 MHz, CDCl₃); □=1.44 (3H), 1.58 (3H), 2.09 (1H), 2.20 (1H), 2.65 (1H), 2.95-3.27 (3H), 3.88 (3Hh), 5.99 (1H), 6.34 (1H), 6.85-7.08 (3H), 7.32 (1H), 7.99 (1H).

Then, a racemate cleavage was performed. After chromatography on a chiral column (Chiralpak AD 20□, mobile solvent hexane/ethanol), the two enantiomers were obtained.

(−)-Enantiomer: Example 100;

(+)-Enantiomer: Example 101.

### Example 101

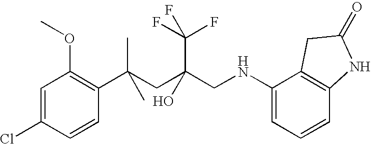

### (+)-4-[4-(4-Chloro-2-methoxy-phenyl)-2-hydroxy-4-methyl-2-trifluoromethyl-pentylamino]-1,3-dihydro-indol-2-one

For conditions for the racemate cleavage, see Example 101.

### Example 102

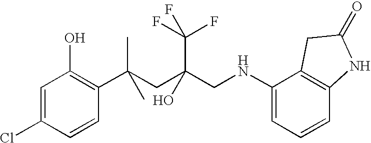

### (−)-4-[4-(4-Chloro-2-hydroxy-phenyl)-2-hydroxy-4-methyl-2-trifluoromethyl-pentylamino]-1,3-dihydro-indol-2-one

The compound was synthesized by ether cleavage of the compound that is described in Example 100.

¹H-NMR (300 MHz, CD₃OD): □=1.32 (3H), 1.49 (3H), 2.43 (1H), 2.59 (1H), 3.10 (1H), 3.21-3.40 (3H), 5.87 (1H), 6.24 (1H), 6.58 (1H), 6.69 (1H), 6.95 (1H), 7.18 (1H).

### Example 103

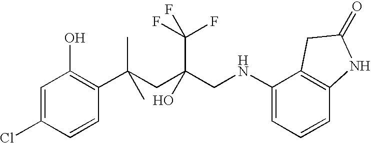

### (+)-4-[4-(4-Chloro-2-hydroxy-phenyl)-2-hydroxy-4-methyl-2-trifluoromethyl-pentylamino]-1,3-dihydro-indol-2-one

The compound was synthesized by ether cleavage of the compound that is described in Example 101. For ¹NMR data, see Example 102.

### Example 104

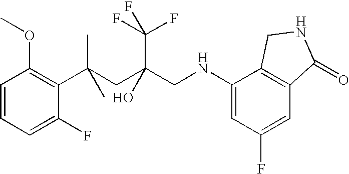

### 6-Fluoro-4-[4-(2-fluoro-6-methoxy-phenyl)-2-hydroxy-4-methyl-2-trifluoromethyl-pentylamino[-2,3-dihydro-isoindol-1-one

The compound was synthesized starting from the corresponding precursors, analogously to what is described in the examples above. 62.5 mg (77.8%) of the desired compound was isolated in the last step.

¹H-NMR (300 MHz, CD₃OD): □=1.53 (3H), 1.79 (3H), 1.98 (1H), 2.95-3.12 (2H), 3.25 (1H), 3.90 (3H), 4.09-4.28 (2H), 6.00 (1H), 6.62 (1H), 6.71 (1H), 6.83 (1H), 7.19 (1H).

### Example 105

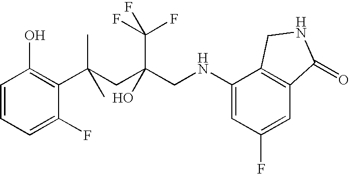

### 6-Fluoro-4-[4-(2-fluoro-6-hydroxy-phenyl)-2-hydroxy-4-methyl-2-trifluoromethyl-pentylamino]-2,3-dihydro-isoindol-1-one

The compound was obtained by ether cleavage of the compound, described in the preceding example, with BBr₃. 37.5 mg (69.7%) of the desired compound was isolated.

¹H-NMR (300 MHz, CD₃OD): □=1.54 (3H), 1.82 (3H), 1.89 (1H), 3.05 (1H), 3.20-3.40 (2H), 4.10-4.28 (2H), 6.05 (1H), 6.47 (1H), 6.58 (1H), 6.70 (1H), 6.98 (1H).

### Example 106

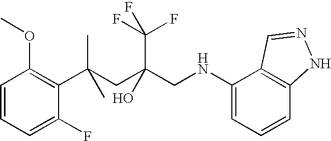

### 1,1,1-Trifluoro-4-(2-fluoro-6-methoxy-phenyl)-2-[(1H-indazol-4-ylamino)-methyl]-4-methyl-pentan-2-ol

The compound was synthesized starting from the corresponding precursors, analogously to what was described in the examples above. 51.2 mg (72.8%) of the desired compound was isolated in the last step.

¹H-NMR (300 MHz, CD₃OD): □=1.54 (3H), 1.80 (3H), 2.03 (1H), 3.00-3.19 (2H), 3.35 (1H), 3.85 (3H), 5.65 (1H), 6.63 (1H), 6.70-6.84 (2H), 7.08 (1H), 7.18 (1H), 7.93 (1H).

### Example 107

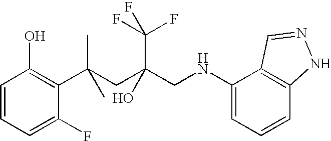

### 1,1,1-Trifluoro-4-(2-fluoro-6-hydroxy-phenyl)-2-[(1H-indazol-4-ylamino)-methyl]-4-methyl-pentan-2-ol

The compound was obtained by ether cleavage of the compound, described in Example 106, with BBr₃. 20.8 mg (54.2%) of the desired compound was isolated.

¹H-NMR (300 MHz, CD₃OD): □=1.57 (3H), 1.80 (3H), 1.92 (1H), 3.15 (1H), 3.20-3.50 (2H), 5.70 (1H), 6.40-6.60 (2H), 6.75 (1H), 6.88-7.10 (2H), 7.90 (1H).

1. Compounds of general formula I 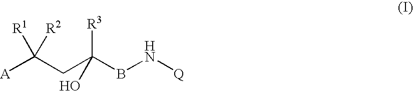
in which
A stands for an aryl, a benzyl or a phenethyl group, whereby the aryl, benzyl or phenethyl group optionally can be substituted by one or more radicals from the group C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —(CH₂)ₙ—CH₂—, —O—CH═CH—, or —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom mean the chain of a C₃-C₆-cycloalkyl ring,
R³ means a C₁-C₈-alkyl group that is optionally substituted, independently of one another, by one or more groups selected from halogen, hydroxy or C₁-C₃-alkoxy, or an optionally partially or completely fluorinated C₁-C₃-alkyl group, an optionally substituted group that is selected from C₂-C₆-alkenyl, C₂-C₆-alkinyl, C₃-C₈-cycloalkyl, C₃-C₇-heterocyclyl, aryl, heteroaryl, (C₁-C₈-alkyl)C₃-C₈-cycloalkyl, (C₁-C₈-alkyl)aryl, or (C₁-C₈-alkyl)heteroaryl,
B means a methylene group or a carbonyl group that is optionally substituted by a methyl or ethyl group, and
Q means a quinazolinyl, quinoxalinyl, cinnolinyl, indazolyl, phthalazinyl, naphthyridinyl, benzothiazolinyl, dihydroindolonyl, dihydroisoindolonyl, benzimidazole or indolyl group that is linked via any position and that optionally can be substituted by one or more radicals from the group C₁-C₅-alkyl, C₁-C₅-alkoxy, C₁-C₅-alkylthio, C₁-C₅-perfluoroalkyl, halogen, hydroxy, cyano, nitro, or NR⁴R⁵, whereby R⁴ and R⁵, independently of one another, can be hydrogen, C₁-C₅-alkyl or (CO)—C₁-C₅-alkyl, whereby phthalazinones are excluded,
as well as their racemates or separately present stereoisomers, and optionally their physiologically compatible salts.

2. Compounds of general formula I according to claim 1, in which
A stands for an aryl group, a benzyl group or a phenethyl group, whereby the aryl, benzyl or phenethyl group optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂—, —O—CH═CH—, or —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom of the chain mean a C₃-C₆-cycloalkyl ring,
R³ means a C₁-C₈-alkyl group that is optionally substituted, independently of one another, by one or more groups that are selected from halogen, hydroxy or C₁-C₃-alkoxy, or a C₁-C₃-alkyl group that is optionally partially or completely fluorinated,
B means a methylene group or a carbonyl group that is optionally substituted by a methyl or ethyl group, and
Q means a quinazolinyl, quinoxalinyl, cinnolinyl, phthalazinyl, naphthyridinyl, benzothiazolyl, indazolyl, dihydroindolonyl, dihydroisoindolonyl, benzimidazole or indolyl group that is linked via any position and that optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro or NR⁴R⁵, whereby R⁴ and R⁵, independently of one another, can be hydrogen, C₁-C₅-alkyl or (CO)—C₁-C₅-alkyl, whereby phthalazinones are excluded,
as well as their racemates or separately present stereoisomers, and optionally their physiologically compatible salts.

3. Compounds of general formula I according to claim 1, in which
A stands for an aryl group, a benzyl group or a phenethyl group, whereby the aryl, benzyl or phenethyl group optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂, —O—CH═CH—, —(CH₂)ₙ₊₂—, whereby n=1 or 2, and the terminal oxygen atoms and/or carbon atoms are linked to directly adjacent ring-carbon atoms, or NR⁴R⁵, whereby R⁴ and R⁵, independently of one another, can be hydrogen, C₁-C₅-alkyl or (CO)—C₁-C₅-alkyl,
R¹ and R², independently of one another, mean a hydrogen atom, a methyl or ethyl group, or together with the carbon atom of the chain mean a C₃-C₆-cycloalkyl ring,
R³ means a C₁-C₃-alkyl group or an optionally partially or completely fluorinated C₁-C₃-alkyl group,
B means a methylene group or a carbonyl group that is optionally substituted by a methyl or ethyl group, and
Q means a quinazolinyl, quinoxalinyl, cinnolinyl, indazolyl, phthalazinyl, naphthyridinyl or benzothiazolyl group that is linked via any position, which optionally can be substituted by one or more radicals from the group of C₁-C₅-alkyl, C₁-C₅-alkoxy, C₁-C₅-alkylthio, C₁-C₅-perfluoroalkyl, halogen, hydroxy, cyano, nitro or NR⁴R⁵, whereby R⁴ and R⁵, independently of one another, can be hydrogen, C₁-C₅-alkyl or (CO)—C₁-C₅-alkyl, whereby phthalazinones are excluded, as well as their racemates or separately present stereoisomers, and optionally their physiologically compatible salts.

4. Compounds according to one of the preceding claims, characterized in that A means an optionally substituted phenyl group.

6. Compounds according to the preceding claims, wherein A is a phenyl radical that is substituted by a hydroxy group or a methoxy group and a halogen atom.

6. Compounds according to one of the preceding claims, wherein Q means a benzothiazolyl, quinazolinyl, quinoxalinyl, cinnolinyl, indazolyl, phthalazinyl or a 1,7- or 1,8-naphthyridinyl group that is linked via any position.

7. Compounds according to claim 1, wherein Q means a benzothiazolyl, quinazolinyl, quinoxalinyl, cinnolinyl, phthalazinyl, 1,7- or 1,8-naphthyridinyl, indazolyl, dihydroindolonyl, dihyroisoindolonyl, benzimidazolyl or indolyl group that is linked via any position.

8. Compounds according to the preceding claims, wherein these are the (+)-enantiomers.

9. Compounds according to the preceding claims, wherein these are the (−)-enantiomers.

10. Process for the production of the compounds of general formula I, wherein for the case that B=CO,
A, a □-keto acid of formula (II)
in which A, R¹ and R², independently of one another, mean a hydrogen atom, a methyl or ethyl group or together with the carbon atom of the chain mean a C₃-C₆-cycloalkyl ring,
is reacted with an amine of formula
Q¹-NH₂
in which Q¹ means benzothiazole, quinazoline, quinoxaline, cinnoline or phthalazine,
to form □-ketoamide (III) 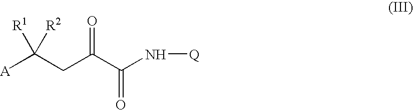
optionally in the presence of dehydrating coupling reagents or after the acid function is activated in the way that is known to one skilled in the art,
which then either is reacted with alkyl metal compounds of general formula (IVa)
R³-M
in which M stands for an alkali metal, MgX or ZnX, X stands for halogen and R³ stands for a C₁-C₈-alkyl group that is optionally substituted, independently of one another, by one or more groups selected from halogen, hydroxy or C₁-C₃-alkoxy, or an optionally partially or completely fluorinated C₁-C₃-alkyl group, an optionally substituted group that is selected from C₂-C₆-alkenyl, C₂-C₆-alkinyl, C₃-C₈-cycloalkyl, C₃-C₇-heterocyclyl, aryl, heteroaryl, (C₁-C₈-alkyl)C₃-C₈-cycloalkyl, (C₁-C₈-alkyl)aryl, or (C₁-C₈-alkyl)heteroaryl group, or
is reacted with a silicon compound of formula (IV)
(R⁶)₃—Si—R³
in which R⁶ means a C₁-C₅-alkyl group and R³ has the above-indicated meanings, or
B, a □-keto acid (II) is esterified according to the method according to one skilled in the art, the □-keto ester (V) is then reacted as described in A with an alkyl metal compound (IVa) or a silicon compound of formula (IV), optionally the ester is cleaved according to the method that is known to one skilled in the art, and a compound of formula (VI) 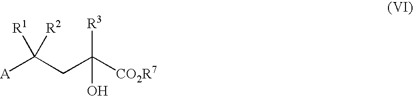
is obtained,
in which R⁷ means C₁-C₅-alkyl or hydrogen, and the latter then is reacted with an amine of formula
Q²-NH₂
in which Q² means quinazolinyl, quinoxalinyl, cinnolinyl, indazolyl, phthalazinyl, naphthyridinyl, benzothiazolyl, dihydroindolonyl, dihydroisoindolonyl, benzimidazolyl or indolyl,
optionally after activation of the acid function and/or optionally in the presence of a catalyst, or
for the case that B means a methylene group that is optionally substituted by a methyl or ethyl group, either a compound of formula (VII) or (VIII) 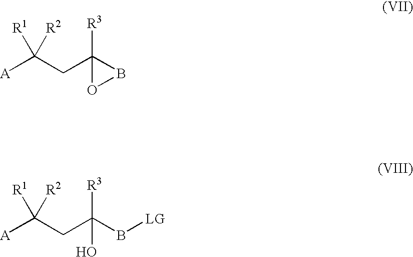
in which A, B, R¹, R², and R³ have the above-indicated meanings, and LG means any leaving group,
is reacted with a compound of formula (IX) or (X)
Q-NH—R⁹ (IX)
Q-N═C═O (X)
in which R⁹ means a hydrogen atom, a C₁-C₅-acyl group or a C₁-C₅-alkoxy group or an aryloxycarbonyl group, and
Q means a quinazolinyl, quinoxalinyl, cinnolinyl, indazolyl, phthalazinyl, naphthyridinyl, benzothiazolyl, dihydroindolonyl, dihydroisoindolonyl, benzimidazole or indolyl group,
and an optionally intermediately formed oxazolidinone is cleaved with aqueous alkali hydroxides, or
compounds of formulas (VII) or (VIII) are reacted with azide salts or ammonia, optionally then it is reduced with the reagents that are known to one skilled in the art, or a transition metal-catalyzed hydrogenation is performed to obtain compounds of formula (XI) 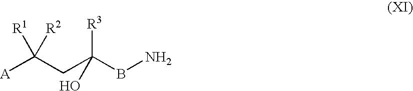
which then optionally is reacted under base catalysis or transition-metal catalysis with a derivative of the heterocyclic compounds quinazoline, quinoxaline, cinnoline, indazole, phthalazine, naphthyridine, benzothiazole, dihydroindolone, dihydroisoindolone, benzimidazole or indole that is halogenated according to the method known to one skilled in the art, or
by compounds of formulation (XII), produced according to the methods known to one skilled in the art from compound (VI) by means of reduction or alkylation,
in which R⁸ means hydrogen, methyl or ethyl,
being reacted under conditions of reductive amination with compounds of the formula
Q-NH₂,
in which Q has the above-indicated meaning.

11. Pharmaceutical preparations that contain at least one compound according to one of the preceding claims or mixtures thereof as well as pharmaceutically compatible vehicles.

12. Use of the compounds according to claim 1 for the production of a pharmaceutical agent.

13. Use of the compounds according to claim 1 for the production of a pharmaceutical agent for treating inflammatory diseases.
